(19) United States
(12) Patent Application Publication
(10) Pub. No.: US 2021/0325406 A1
(43) Pub. Date: Oct. 21, 2021

(54) PROTEIN NANOPORE FOR IDENTIFYING AN ANALYTE

(71) Applicant: NANJING UNIVERSITY, Nanjing, Jiangsu (CN)

(72) Inventors: Shuo HUANG, Nanjing (CN); Jiao CAO, Nanjing (CN)

(21) Appl. No.: 17/272,420

(22) PCT Filed: Aug. 27, 2019
(86) PCT No.: PCT/CN2019/102756

§ 371 Date: Mar. 1, 2021

(30) Foreign Application Priority Data
Aug. 28, 2018 (CN) PCT/CN2018/102709

(51) Int. Cl. G01N 33/68; G01N 33/84; G01N 27/02

(52) U.S. Cl. CPC G01N 33/6872; G01N 27/02; G01N 33/84

(57) ABSTRACT

Use of a metal embedded protein nanopore in identifying an analyte and use of MspA in identifying a metal-containing analyte are provided. Methods and Systems of identifying an analyte using a protein nanopore are also provided. Furthermore, a kit for identifying an analyte is provided, which contains a metal-containing compound and a protein that can form a nanopore.

### FIELD

This invention relates to a method for identifying an analyte using protein nanopore.

### BACKGROUND

A biological nanopore, which is the core component of a commercial sequencer[1], is capable of decoding a tremendous amount of information including length[2], sequence[3,4], base modification[5] from DNA and many other biomacromolecules including RNA[6], peptides[7] and proteins[8]. This remarkable sensing performance originates from its biological role as an ion channel[9]. Since it is the only pathway through which ions can cross the membrane, a biological nanopore could resolve chemical binding of an individual ion within the pore restriction[10], indicating a precision far greater than that of a solid state nanopore[11].

Pioneered by Bayley et al. since 1997[10], nanopore-based direct sensing of single ions such as Co²⁺, Ag⁺ or Cd²⁺ is performed by a designed ion-amino acid coordination[10,12,13] or an ion-chelator interaction[14] within an engineered a-hemolysin (a-HL) mutants. However, α-HL blockages by single monatomic ions of different identities show consistently shallow resistive pulses (˜2-3 pA), resulting from the cylindrical pore geometry and the small size of the analyte ions[10, 12, 13]. Alternatively, indirect sensing of metal ions can be performed with molecular adapters like DNA[15], peptides[16] or cyclodextrins[17] but with diminished signal specificity and an increased system complexity.

Chloroauric acid (HAuCl₄), a well-known gold compound[18], is a precursor that is used widely for the fabrication of gold nanomaterials[18] . In an aqueous buffered solution, the dissociated tetrachloroaurate(III) ion ([AuCl₄]⁻) is a square planar, polyatomic ion with a net charge of −1, in which the Au—Cl bond measures 2.28 Å in length[20]. Previous reports indicated that the tetrachloroaurate(III) ion is a potent aquaporin inhibitor[21] but investigations at the level of single molecules have not been reported.

### SUMMARY

One aspect of this invention provides use of a metal embedded protein nanopore in identifying an analyte in a sample.

In some embodiments, the metal embedded protein nanopore is a protein nanopore embedded one or more with metal-containing ions.

In some embodiments, the one or more metal-containing ions are selected from the group consisting of ions contain Au(III), Zn²⁺, Co²⁺, Ag²⁺, Cd²⁺, Ni²⁺, Au(I), Cu²⁺, Cr³⁺, Fe²⁺, Fe³⁺, Pt(II), Pd(II), Mn²⁺, Hg²⁺, Ru(II) and Pb²⁺.

In some embodiments, the one or more metal-containing ions are selected from the group consisting of [AuCl₄]⁻, Zn²⁺, Co²⁺, Ag⁺, Cd²⁺, Ni²⁺, [AuCl_{2]}⁻, Cu²⁺, Cr³⁺, Fe²⁺, Fe³⁺, Pt(II), Pd(II), Mn²⁺, Hg²⁺, Ru(II) and Pb²⁺ ions.

In some embodiments, the one or more metal-containing ions are bound to methionine, cysteine, histidine or any combination of them on the inner surface of the nanopore.

In some embodiments, the number of the one or more metal-containing ions which are bound to methionine, cysteine, histidine or any combination of them on the inner surface of the nanopore is 1, 2, 3, 4, 5, 6, 7, or 8.

In some embodiments, the protein nanopore is a-HL or MspA, CsgG, OmpG, Cytolysin A, ClyA, aerolysin, Frac or Phi29 connector.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

In some embodiments, the analyte is metal-containing analyte such as metal-containing ion or nucleic acid.

In some embodiments, the analyte is amino acid or peptide; preferably, the amino acid or peptide contains sulfur.In some embodiments, the amino acid contains one or more sulfur atoms on the amino acid side chain or contains thiol group; more preferable, the amino acid is L-methionine, L-cysteine or L-homocysteine. In some embodiments, the peptide contains an amino acid containing one or more sulfur atoms on the amino acid side chain or an amino acid containing thiol group; more preferable, the peptide contains L-methionine, L-cysteine or L-homocysteine.

In some embodiments, the analyte is a thiol; preferably, the analyte is a biothiol; more preferably, the biothiol is L-cysteine, L-homocysteine, and/or L-glutathione.

In some embodiments, the nucleic acid is ssDNA, dsDNA, RNA, or a combination thereof.

Another aspect of this invention provides a method of identifying an analyte in a sample is provided, the method comprising:

providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample, wherein the nanopore is embedded with one or more metal-containing ions;

applying an electric field across the nanopore and translocating the analyte through the nanopore;

measuring the blockade current across the nanopore; and

identifying the analyte according to the measured blockade current.

In some embodiments, the one or more metal-containing ions are selected from the group consisting of ions contain Au(III), Zn²⁺, Co²⁺, Ag⁺, Cd²⁺, Ni²⁺, Au(I), Cu²⁺, Fe²⁺, Fe³+, Pt(II), Pd(II), Mn²⁺, Hg²⁺, Ru(II) and Pb²⁺.

In some embodiments, the one or more metal-containing ions are selected from the group consisting off ions.

In some embodiments, the one or more metal-containing ions are bound to methionine, cysteine, histidine or any combination of them on the inner surface of the nanopore.

In some embodiments, the number of the one or more metal-containing ions which are io bound to methionine, cysteine, histidine or any combination of them on the inner surface of the nanopore is 1, 2, 3, 4, 5, 6, 7, or 8.

In some embodiments, the protein nanopore is a-HL or MspA, CsgG, OmpG, Cytolysin A, ClyA, aerolysin, Frac or Phi29 connector.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

In some embodiments, the analyte is metal-containing analyte such as metal-containing ion or nucleic acid.

In some embodiments, the analyte is amino acid or peptide; preferably, the amino acid or peptide contains sulfur. In some embodiments, the amino acid contains one or more sulfur atoms on the amino acid side chain or contains thiol group; more preferable, the amino acid is L-methionine, L-cysteine or L-homocysteine. In some embodiments, the peptide contains an amino acid containing one or more sulfur atoms on the amino acid side chain or an amino acid containing thiol group; more preferable, the peptide contains L-methionine, L-cysteine or L-homocysteine.

In some embodiments, the analyte is a thiol; preferably, the analyte is a biothiol; more preferably, the biothiol is L-cysteine, L-homocysteine, and/or L-glutathione.

In some embodiments, the nucleic acid is ssDNA, dsDNA, RNA, or a combination thereof.

In some embodiments, the method comprising:

providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample and metal-containing ions;

applying an electric field across the nanopore and translocating the metal-containing ions and the analyte through the nanopore;

measuring the blockade current across the nanopore; and

identifying the analyte in the sample according to the measured blockade current.

In another embodiment, the method comprising:

providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises metal-containing ions;

applying an electric field across the nanopore and translocating the metal-containing ions through the nanopore;

adding the sample into the conductive liquid medium which comprises the metal-containing contianing ion initially and translocating the analyte through the nanopore;

measuring the blockade current across the nanopore; and

identifying the analyte in the sample according to the measured blockade current.

In some embodiments, the metal-containing ions are one or more selected from the group consisting of ions contain Au(III), Zn²⁺, Co²⁺, Ag⁺, Cd²⁺, Ni²⁺, Au(I), Cu²⁺, Fe²⁺, Fe³⁺, Pt(II), Pd(II), Mn²⁺, Hg²⁺, Ru(II) and Pb²⁺.

In some embodiments, the metal-containing ions are one or more selected from the group consisting of [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) and Pb²⁺ ions.

In some embodiments, the protein nanopore is α-HL or MspA, CsgG, OmpG, Cytolysin A, ClyA, aerolysin, Frac or Phi29 connector.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of

D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

In some embodiments, the analyte is metal-containing analyte such as metal-containing ion or nucleic acid.

In some embodiments, the analyte is amino acid or peptide; preferably, the amino acid or peptide contains sulfur. In some embodiments, the amino acid contains one or more sulfur atoms on the amino acid side chain or contains thiol group; more preferable, the amino acid is L-methionine, L-cysteine or L-homocysteine. In some embodiments, the peptide contains an amino acid containing one or more sulfur atoms on the amino acid side chain or an amino acid containing thiol group; more preferable, the peptide contains L-methionine, L-cysteine or L-homocysteine.

In some embodiments, the analyte is a thiol; preferably, the analyte is a biothiol; more preferably, the biothiol is L-cysteine, L-homocysteine, and/or L-glutathione.

In some embodiments, the nucleic acid is ssDNA, dsDNA, RNA, or a combination thereof.

Another aspect of this invention provides a system of identifying an analyte in a sample, the system contains a protein nanopore positioned between a first conductive liquid medium and a second conductive liquid medium, wherein the nanopore comprises an opening that is provides liquid communication between the first conductive liquid medium and the second conductive liquid medium, wherein the first conductive liquid medium or the second conductive liquid medium comprises the sample, wherein the nanopore is embedded with one or more metal-containing ion.

In some embodiments, the one or more metal-containing ions are selected from the group consisting of ions contain Au(III), Zn²⁺, Co²⁺, Ag⁺, Cd²⁺, Ni²⁺, Au(I), Cu²⁺, Fe²⁺, Fe³⁺, Pt(II), Pd(II), Mn²⁺, Hg²⁺, Ru(II) and Pb²⁺.

In some embodiments, the one or more metal-containing ions are selected from the group consisting of [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) and Pb²⁺ ions.

In some embodiments, the one or more metal-containing ions are bound to methionine, cysteine, histidine or any combination of them on the inner surface of the nanopore.

In some embodiments, the number of the one or more metal-containing ions which are bound to methionine, cysteine, histidine or any combination of them on the inner surface of the nanopore is 1, 2, 3, 4, 5, 6, 7, or 8.

In some embodiments, the protein nanopore is a-HL or MspA, CsgG, OmpG, Cytolysin A, ClyA, aerolysin, Frac or Phi29 connector.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

In some embodiments, the analyte is metal-containing analyte such as metal-containing ion or nucleic acid.

In some embodiments, the analyte is amino acid or peptide; preferably, the amino acid or peptide contains sulfur. In some embodiments, the amino acid contains one or more sulfur atoms on the amino acid side chain or contains thiol group; more preferable, the amino acid is L-methionine, L-cysteine or L-homocysteine. In some embodiments, the peptide contains an amino acid containing one or more sulfur atoms on the amino acid side chain or an amino acid io containing thiol group; more preferable, the peptide contains L-methionine, L-cysteine or L-homocysteine.

In some embodiments, the analyte is a thiol; preferably, the analyte is a biothiol; more preferably, the biothiol is L-cysteine, L-homocysteine, and/or L-glutathione.

In some embodiments, the nucleic acid is ssDNA, dsDNA, RNA, or a combination thereof.

In some embodiments, the system contains a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample and metal-containing ions.

In another embodiment, the system contains a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises metal-containing ions.

In some embodiments, the metal-containing ions are one or more selected from the group consisting of ions contain Au(III), Zn²⁺, Co²⁺, Ag⁺, Cd²⁺, Ni²⁺, Au(I), Cu²⁺, Fe²⁺, Fe³⁺, Pt(II), Pd(II), Mn²⁺, Hg²⁺, Ru(II) and Pb²⁺.

In some embodiments, the metal-containing ions are one or more selected from the group consisting of [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) and Pb²⁺ ions.

In some embodiments, the protein nanopore is α-HL or MspA, CsgG, OmpG, Cytolysin A, ClyA, aerolysin, Frac or Phi29 connector.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

In some embodiments, the analyte is metal-containing ion analyte such as metal-containing or nucleic acid.

In some embodiments, the analyte is amino acid or peptide; preferably, the amino acid or peptide contains sulfur. In some embodiments, the amino acid contains one or more sulfur atoms on the amino acid side chain or contains thiol group; more preferable, the amino acid is L-methionine, L-cysteine or L-homocysteine. In some embodiments, the peptide contains an amino acid containing one or more sulfur atoms on the amino acid side chain or an amino acid containing thiol group; more preferable, the peptide contains L-methionine, L-cysteine or L-homocysteine.

In some embodiments, the analyte is a thiol; preferably, the analyte is a biothiol; more is preferably, the biothiol is L-cysteine, L-homocysteine, and/or L-glutathione.

In some embodiments, the nucleic acid is ssDNA, dsDNA, RNA, or a combination thereof.

Another aspect of this invention provides a kit for identifying an analyte, the kit containing: (1) metal-containing compound; and (2) a protein that can form a nanopore or a nucleic acid, expression vector or recombinant host cell that can express a protein that can form a nanopore; said metal-containing compound is capable of forming metal-containing ions in a solution.

In some embodiments, the metal-containing compound contains Au(III), Zn²⁺, Co²⁺, Ag⁺, Cd²⁺, Ni²⁺, Au(I), Cᵤ²⁺, Cr³⁺, Fe²⁺, Fe³⁺, Pt(II), Pd(II), Mn²⁺, Hg²⁺, Ru(II) or Pb²⁺.

In some embodiments, the metal-containing compound is capable of forming [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) or Pb²⁺ ions in a solution.

In some embodiments, the metal-containing compound may be chloroauric acid or tetrachloroaurate(III).

In some embodiments, the protein is a-HL or MspA, CsgG, OmpG, Cytolysin A, ClyA, aerolysin, Frac or Phi29 connector.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

In some embodiments, the analyte is metal-containing analyte such as metal-containing ion or nucleic acid. In some embodiments, the analyte is amino acid or peptide; preferably, the amino acid or peptide contains sulfur. In some embodiments, the amino acid contains one or more sulfur atoms on the amino acid side chain or contains thiol group; more preferable, the amino acid is L-methionine, L-cysteine or L-homocysteine. In some embodiments, the peptide contains an amino acid containing one or more sulfur atoms on the amino acid side chain or an amino acid containing thiol group; more preferable, the peptide contains L-methionine, L-cysteine or L-homocysteine.

In some embodiments, the analyte is a thiol; preferably, the analyte is a biothiol; more preferably, the biothiol is L-cysteine, L-homocysteine, and/or L-glutathione.

In some embodiments, the nucleic acid is ssDNA, dsDNA, RNA, or a combination thereof.

Another aspect of this invention provides use of MspA in identifying a metal-containing ion in a sample.

In some embodiments, the metal-containing ion are selected from the group consisting of ions contain Au(III), Zn²⁺, Co²⁺, Ag⁺, Cd²⁺, Ni²⁺, Au(I), Cu²⁺, Cr³⁺, Fe²⁺, Fe³⁺, Pt(II), Pd(II), Mn²⁺, Hg²⁺, Ru(II) and Pb²⁺.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

Another aspect of this invention provides method of identifying a metal-containing ion in a sample, the method comprising:

providing a MspA positioned between a first conductive liquid medium and a second conductive liquid medium, wherein the MspA comprises an opening that provides liquid communication between the first conductive liquid medium and the second conductive liquid medium, wherein the first conductive liquid medium or the second conductive liquid medium comprises the sample;

applying an electric field across the MspA and translocating the metal-containing ion through the nanopore;

measuring the blockade current across the nanopore; and

identifying the metal-containing ion in the sample according to the measured blockade current.

In some embodiments, the metal-containing ion are selected from the group consisting of ions contain Au(III), Zn²⁺, Co²⁺, Ag⁺, Cd²⁺, Ni²⁺, Au(I), Cu²⁺, Cr³⁺, Fe²⁺, Fe³+, Pt(II), Pd(II), Mn²⁺, Hg²⁺, Ru(II) and Pb²⁺.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface.

In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

Another aspect of this invention provides system of identifying a metal-containing ion in a sample, the system contains a MspA positioned between a first conductive liquid medium and a second conductive liquid medium, wherein the MspA comprises an opening that provides liquid communication between the first conductive liquid medium and the second conductive liquid medium, wherein the first conductive liquid medium or the second conductive liquid medium comprises the sample.

In some embodiments, the metal-containing ion are selected from the group consisting of ions contain Au(III), Zn²⁺, Co²⁺, Ag⁺, Ni²⁺, Au(I), Cu²⁺, Cr³⁺, Fe²⁺, Fe³+, Pt(II), Pd(II), Mn²⁺, Hg²⁺, Ru(II) and Pb²⁺.

In some embodiments, MspA is a mutant octameric MspA, said mutant octameric MspA comprises at least one mutant MspA monomers, said mutant octameric MspA has no spontaneous gating activities at positive voltages and has methionine, cysteine, histidine or any combination of them on the inner surface. In some embodiments, at least one of the mutant MspA monomers comprises one or more mutations at positions 83-111 compared to the wild-type MspA monomer; preferably, the one or more mutations at positions 83-111 may be one or more mutations at position 88, 89, 90, 91, 92, 93, 94, 95, 96, 97, 98, 99, 100, 101, 102, 103, 104 and/or 105; more preferably, at least one of the mutant MspA monomers comprises mutation at positions 88, 91 and/or 105 compared to the wild-type MspA monomer; more preferably, said mutation is a mutation to methionine, cysteine or histidine; more preferably, at least one of the mutant MspA monomers comprises the mutation of D91M, D91H or D91C compared to the wild-type MspA monomer; more preferably, at least one of the mutant MspA monomers comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer.

### DESCRIPTION OF THE DRAWINGS

FIG. 1 shows [AuCl4]⁻¹ binding within a WT α-HL nanopore. (a) Heptameric WT α-HL and its sensing mechanism for [AuCl4]⁻¹ ions. Each heptameric WT α-HL nanopore has seven identical methionine residues (yellow in the structural diagram) at position 113. A reversible coordination interaction takes place between a single [AuCl4]⁻¹ and any one of the seven methionine (M113) residues. (b) A representative trace with all-points histogram for [AuCl4]⁻¹ sensing by WT α-HL. The electrophysiology recording was carried out in 1.5 M KCl buffer at +100 mV with a final concentration of 5 μM HAuCl₄ cis (Methods). Continuous pore blockages by single [AuCl4]⁻¹ are clearly resolved with consistent blockage amplitude (ΔI≈6 pA). (c) A representative trace with all-points histogram for [AuCl4]⁻¹ sensing by WT α-HL. The electrophysiology recording was carried out in 1.5 M KC1 buffer at +100 mV with a final is concentration of 15 μM HAuCl₄ in cis (Methods). At this concentration, sequential binding from multiple [AuCl4]⁻¹ ions within the same WT α-HL is observed. Here, M—(AuCl₄⁻¹)ₙ stands for n [AuCl4]⁻¹ currently in the pore. Direct transition between M—(AuCl₄⁻¹)ₙ and M—(AuCl₄⁻)_{n±2} is never observed. (d) The event histogram from 10 min recording of [AuCl4]⁻¹ binding in WT α-HL with 15 μm HAuCl₄ in cis and +100 mV applied potential. Gaussian fitting is performed for binding events from different numbers of [AuCl4]⁻¹ ions and marked as M—AuCl₄⁻, M-(AuCl₄⁻)₂, and M—(AuCl₄⁻)₃ (M—AuCl₄⁻: 6.28±1.03 pA, N=179; M≤(AuCl₄⁻)₂:12.21±0.99 pA, N=75; M—(AuCl₄⁻)₃:18.88±0.99 pA, N=12).

FIG. 2 shows multiple [AuCl4]⁻¹ binding events within an α-HLWT nanopore. (a-c) All electrophysiology recordings were carried out in 1.5 M KCl buffer at +100 mV with a final concentration of 5-15 μM HAuCl₄ cis (Methods). (d-f) Scatter plot of ΔI vs. dwell time for different HAuCl₄ concentrations (1 μM, 10 μM and 15 μM). Data for all scatter plots were from 10 min continuous electrophysiology recording for each condition. A wide dispersion of blockage amplitude is clearly observed in f.

FIG. 3 shows purification and characterization of hetpameric α-HL M113G. Monomeric α-H LM113G was expressed with E Coli. BL21 (DE3) and purified by nickel affinity chromatography (Methods).[51] Heptameric α-HL M113G spontaneously forms after cell lysis. The monomeric and heptameric α-HL M113G can be isolated based on the difference of binding affinity between α-HL M113G monomer and heptamer with the nickel column. (a) The UV absorbance spectrum during gradient elution (0 mM to 300 mM of immdiazole) for the supernatant of the cell lysate. Identities of the eluted samples were determined in the corresponding gel electrophoresis. (b) Gel electrophoresis of eluted protein with 7.5% SDS-polyacrylamide gel. Lanes: M, precision plus protein standards (Bio-Rad); 1-5, the corresponding elution fractions in (a). Lane 2 was confirmed to be the α-HL M113G monomer and lane 3, 4, 5 were confirmed to be the α-HL M113G heptamer respectively, based on previously published results with α-HL WT[51]. Purified α-HL M113G heptamer could be directly used for electrophysiology measurements or stored at −80° C. for long term storage. (c) IV curves from heptameric α-HL WT and α-HL M113G. Both IV curves were collected with 1.5 M KCl buffer (1.5 M KCl, 10 mM Tris-HCl, pH=7.0). Slight variation of IV curves between α-HL WT and M113G was systematically observed whereas the general conductance of M113G mutant remains.

FIG. 4 shows background current of α-HL M113G with or without HAuCl₄. The background current was recorded in 1.5 M KCl buffer at +100 mV (Methods) with or without HAuCl₄ in cis. (a) Background current recorded using α-HL M113G without HAuCl₄. At +100 mV, α-HL M113G stays open with no spontaneous gating, which indicates that an unaltered heptameric pore assembly forms after the mutation. (b) Background current recorded using α-HL M113G with HAuCl₄ in cis reaching a 50 μM final concentration. Continuous recording at +100 mV showed no [AuCl₄]⁻ ion binding event at all due to the M113G mutation, where the side group of the introduced glycine doesn't establish any detectable interaction with the tetrachloroaurate(III). This phenomenon confirms that tetrachloroaurate(III) sensing as observed in WT α-HL originates from the coordination interaction between Au(III) and methionine 113.

FIG. 5 shows background current of M2 MspA with or without HAuCl₄. The background current was recorded in 1.5 M KCl buffer at +100 mV (Methods) with or without HAuCl₄ in cis. (a) Background current recorded using M2 MspA without HAuCl₄. M2 MspA stays open at +100 mV with no spontaneous gating. (b) Background current recorded using M2 MspA with HAuCl₄ reaching a 50 μM final concentration. Continuous recording at +100 mV showed no [AuCl4]⁻¹ binding event in M2 MspA due to lack of sulfur-containing amino acid residues (methionine or cysteine) in the vicinity of the pore restriction. Thus, M2 MspA, which is free of [AuCl4]⁻¹ binding, serves as an ideal pore template for methionine introduction.

FIG. 6 shows reversible and sequential binding of [AuCl4]⁻¹ with methionine (D91M) within an engineered MspA nanopore (MspA-M). (a) The structure of MspA (PDB ID: luun)[22] and its sensing mechanism for [AuCl₄]⁻ ions. Pore engineering (D93N/D91M/D9ON/D118R/D134R/E139K) was performed according to published methods[20] with the exception of D91M for [AuCl4]⁻¹ sensing. The mutant MspA (MspA-M) possesses eight identical methionine residues at position 91, and capable of binding multiple [AuCl4]⁻¹ ions simultaneously. (b, c) Representative traces with all-points histogram for [AuCl4]⁻¹ sensing by MspA-M at +100 mV with 1 μM and 10 μM HAuCl₄ in cis, respectively. With 1 μM HAuCl₄ in cis (b), most binding events are from single [AuCl4]⁻¹ ions, but with 10 μMM HAuCl₄ in cis (c), sequential binding events from multiple [AuCl4]⁻¹ dominate. M-(AuCl₄⁻)ₙ stands for n [AuCl4]⁻¹ ions simultaneously in the pore. (d) Event histogram for 5 min recording for [AuCl4]⁻¹ binding by MspA-M with 10 μM HAuCl₄in cis. Gaussian fitting is performed for binding events from different number of [AuCl₄]⁻¹ and marked as M-AuCl₄⁻, M-(AuCl₄⁻)₂, and M-(AuCl₄⁻)₃ respectively. (M-AuCl₄⁻): 11.31±0.20 pA, N=280; M-(AuCl₄⁻)₂: 23.95±0.29 pA, N=94; M-(AuCl₄⁻)3: 40.09±0.16 pA, N=13). (e) Plot of the reciprocals of the mean inter-event intervals (τₒₙ) and plot of the reciprocals of the mean residence time (τ_{off}) for single [AuCl₄]⁻¹ binding events versus [AuCl4]⁻¹ ions concentration in cis. An abrupt increase in the event detection rates is observed at 1 μM. Further increase of HAuCl₄ concentration in cis leads to more sequential binding from multiple [AuCl4]⁻¹ (FIG. 8). Mean±Standard Deviation of τₒₙ, τ_{off} are from three independent experiments (N=3) with 10 min recording for each condition.

FIG. 7 shows purification and characterization of Octameric MspA-M. Monomeric MspA-M is expressed with E Coli. BL21 (DE3) and purified by nickel affinity chromatography (Methods)[51]. Octameric MspA-M self assembles immediately after cell lysis. The monomeric and octameric MspA-M can be isolated based on their different binding affinity with the nickel column. (a) The UV absorbance spectrum during gradient elution (0 mM to 300 mM of immdiazole) for the supernatant of the cell lysate. Identities of the eluted samples are determined in the corresponding gel electrophoresis. An obvious peak, which appears between 16 to 20 mL in elution volume, is from non-specific protein in the cell lysis. Though barely noticeable, the region, which is marked with 1-4, indicates where the octameric MspA-M are expected to be eluted based on the previous experience with M2 MspA purification. [51] (b) Gel electrophoresis of different elution fractions on a 7.5% SDS-polyacrylamide gel. Lanes: M, precision plus protein standards (Bio-Rad); 1-4, the corresponding elution fractions in (a). io Although barely visible, Lane 1-3 is confirmed to contain octameric MspA-M by taking M2 MspA as a marker. Purified octameric MspA-M could be directly used for electrophysiology measurements or stored at −80° C. for long term storage. The image inset is contrast adjusted so that the band could be seen. (c) IV curves of M2 MspA and MspA-M nanopore. Both IV curves are collected in 1.5 M KCl buffer (1.5 M KCl, 10 mM Tris-HCl, pH=7.0). Slight variation of IV curves between M2 MspA and MspA-M is systematically observed whereas the general conductance of MspA-M retains with no noticeable gating, indicating a successful mutant pore construction.

FIG. 8 shows binding of multiple [AuCl4]⁻¹ ions in MspA-M. Sequential and reversible blockages by multiple [AuCl4]⁻¹ ions within the same MspA-M nanopore show similar blockage spacing. Direct transitions between M—(AuCl₄⁻¹)ₙ and M-(AuCl₄⁻)_{n±2} are never observed. Here M-(AuCl4⁻)₀ stands for the open pore level I. The electrophysiology recording was performed in 1.5 M KCl buffer with +100 mV

FIG. 9 shows dwell time analysis. All mean dwell time in this invention is derived according to this definition if not otherwise stated. (a) An example current-time trace of tetrachloroaurate(III) binding. τₒₙ represents the inter-event duration time. (b) Histograms of the inter-event duration time (τₒₙ) with single exponential fitting. the black line is an exponential fitting according to the equation y=y0+A*exp (−x/τ), the mean inter-event intervals (τₒₙ) is derived from the fitting parameter T.

FIG. 10 shows [AuCl4]⁻¹ binding kinetics within an MspA-M nanopore. (a-c) Representative current recordings with different HAuCl₄ concentrations (1 μM, 5 μM and 10 μMM). (d-f) Corresponding blockage event histograms with different HAuCl₄ concentrations. (g-i) Histograms of the mean inter-event intervals (iₒn) with different HAuCl₄ concentrations. All statistics (d-i) are performed from continuous electrophysiology recording with 280 s in each condition. (j) Plot of the reciprocals of the mean inter-event intervals (τₒₙ) versus HAuCl₄ ions concentration. The increased 1/Tₒn values with increasing HAuCl₄ concentrations confirm that the event results from tetrachloroaurate(III) binding, where a higher HAuC1₄ concentration leads to the more frequent analyte binding. (k) Log dwell-time histograms for M-AuCl₄⁻, M-(AuCl₄⁻)₂, and M-(AuCl₄)₃ in the presence of 10 μM HAuCl₄ ions. The mean and standard deviation of the dwell times are 295.81±276.44 ms, 120.82±117.39 ms, 52.72±41.79 ms, respectively. The much reduced mean dwell time for M-(AuCl₄⁻)₃ means that the probability for the pore to simultaneously accommodate 3 tetrachloroaurate(III) is small.

FIG. 11 shows a comparison between WT α-HL and MspA-M for single [AuCl4]⁻¹ sensing. (a) Geometric comparison between WT α-HL (red, mushroom-shaped) and MspA-M (blue, conical). Yellow spots (central, four spots) indicate the location of methionine on the pore. (b) Representative binding events from a single [AuCl₄]⁻ within a WT α-HL or a MspA-M nanopore at +100 mV, respectively. (c) Event-amplitude (AI) histograms with Gaussian fitting for single [AuCl₄]⁻ bindings within a WT α-HL (red, left) or an MspA-M (blue, right) nanopore. The [AuCl4]⁻¹ sensing performance is significantly improved with MspA-M showing a deeper blockage depth and a narrower distribution width (WT α-HL: 5.63 ±0.33 pA; MspA-M: 11.25±0.19 pA). (d) Log dwell-time histograms for WT α-HL (red, darker) and MspA-M (blue, lighter) for single [AuCl4]⁻¹ bindings. Single [AuCl4]⁻¹ binding within MspA-M shows a systematically reduced event dwell time, possibly due to a sharper restriction from the conical MspA pore (WT a-HL: 12.30±19.23 s ; MspA-M: 0.40±0.36 s). The statistical data in (c) and (d) were taken from 10 min continuous electrophysiology recording at +100 mV with 1 μM HAuChin cis. (e-g) Representative traces for [AuCl4]⁻¹ ions binding in MspA-M at +20 mV (e), +100 mV (f) and +180 mV (g) with 1 μM HAuCl₄ in cis. All traces (e-g) were digitally filtered with a 200 Hz low-pass Bessel filter (eight-pole) by Clampfit so that the shallow binding events in (e) could be presented. (h) Plot of the mean blockage depth for [AuCl₄]⁻ions is in MspA-M and WT α-HL at different voltages (Supplementary Table S5). The statistical data are from three independent experiments (N=3) with 15 min recording for WT α-HL and 5 min recording for MspA-M at each condition. An extended acquisition time for WT α-HL was taken to compensate the reduced event counts compared to that from MspA-M.

FIG. 12 shows [AuCl₄]⁻ binding in WT α-HL with fluctuations. (a-c) Representative traces for [AuCl4]⁻¹ binding in WT α-HL in 1.5 M KCl at +60 mV (a), +100 mV (b) and +180 mV (c) applied potential with 15 μM HAuChin cis. All recorded traces (a-c) (Methods) were digitally filtered with a 200 Hz low-pass Bessel filter (eight-pole) by Clampfit so that the shallow binding events in (a) and baseline fluctuations in (b) and (c) could be presented. Baseline fluctuations are systematically observed in all electrophysiology recordings with WT α-HL in [AuCl₄]⁻ sensing, when a high potential (>+100 mV) is applied. We conclude that potential non-specific bindings of [AuCl4]⁻¹ within the long, cylindrical restriction of a-HL should contribute to this phenomenon.

FIG. 13 shows representative traces for [AuCl4]⁻¹ binding events in MspA-M. (a) A representative trace for 10 μM HAuCl₄ binding in MspA-M at +100 mV. (b) Numbered blockades from the trace (a) shown at expanded time scales. To better compare the blockade depth between M-AuCl₄⁻, M-(AuCl₄⁻)₂, and M-(AuCl₄⁻)₃, the baselines have been offset shifted. (c) Plot of the mean blockade depth for M-AuCl₄⁻, M-(AuCl₄⁻)₂, and M-(AuCl₄⁻)₃. Blockages of these three levels are approximately equally spaced. (d) Histogram for the peak amplitude differences (ΔIₙ=I_{M-(Aucl4̅)}_{ₙ}−I_{M}-(_{AuC14})ₙ₋₁). The rule 0/₃ >0/₂ >A/₁ is systematically observed, which comes from signal amplification with a further narrowed pore restriction. The statistical results (Mean±Standard Deviation) in (c) and (d) are from three independent experiments (N=3) with 10 min recording using MspA-M for each condition. (Table 5)

FIG. 14 shows finite Element Methods (FEM) simulation. A semi-quantitative study with FEM simulation is built to demonstrate how the electric field strength is amplified with a conical pore with a narrower restriction. The FEM simulation is performed with Comsol 5.3a similar as reported. [51, 52] A Nernst Planck Poisson model is used for the simulation. The model geometry is axis-symmetric to simplify the computation. A membrane with 10 nm thickness is established between the cis and trans chamber filled with 1.5 M KCl buffer. A conical geometry is established through the membrane with 6 nm in diameter at the bigger end and 2 nm in diameter at the restriction. A ring blocker with 0.2 nm in thickness and with varying width (0.2-0.8 nm) is established to narrow the pore restriction. The z-component of the electric field strength is calculated and demonstrated in the model. (a) Z-component electric field strength within the pore geometry with a wide remaining restriction size (1.6 nm in diameter). The electric field strength is widely distributed. (b) Z-component electric field strength within the pore geometry with a narrow remaining restriction size (0.4 nm in diameter). The electric field strength is sharply distributed around the pore restriction. (c) Z-component electric field strengths with different pore restriction sizes. It is clear that the electric field strength show a sharper distribution with narrower pore restriction. (d) Electric io potential along the z axis with different pore restriction sizes.

FIG. 15 shows Enhanced poly(dA)io translocation through [AuCl4]⁻¹ embedded MspA-M. (a) Finite-element simulation of the electric field within a conical nanopore with shrinking restriction. Left: simulated electric field within a wide pore restriction (1.6 nm in diameter). Right: simulated electric field within a narrow pore restriction (0.4 nm in diameter). (FIG. 14) (b) Poly (dA)io translocation through an [AuCl_{4]}⁻¹ embedded MspA-M nanopore. The current trace is recorded at +100 mV with 8 μM poly(dA)₁₀ and 10 μM HAuCl₄ added to cis. Sequential [AuCl_{4]}⁻¹ binding within an MspA nanopore gradually shrinks the pore cavity for enhanced DNA sensing. Level n (n=0-3) indicates n [AuCl4]⁻¹ ions currently embedded within the constriction of MspA-M. Enhanced translocation rate and blockage amplitude of poly (dA)io are observed from level 1 to level 3. (c) A zoomed-in view (200 ms) of poly (dA)₁₀ sensing with different level n is demonstrated in the top row. Scatter plot of the absolute blocked depth vs dwell time for different numbered levels are demonstrated in the bottom row. 1500 events from each level are counted in the statistics. (d) Histograms of the mean inter-event intervals (τₒₙ) for numbered levels with single exponential fitting. Again, 1500 events are counted from each level. Reduced τₒₙ from level 3 indicates enhanced translocation rates from [AuCl_{4]}⁻¹ embedded MspA nanopores. (e-g) Mean ΔI/I₀, dwell time and the reciprocals of the mean inter-event intervals (τₒₙ) from each numbered levels. All means and standard deviations are from three independent experiments (10 min recording, N=3, Table 7).

FIG. 16 shows 78nt ssDNA sensing using MspA-M with Au embedment. (a) A representative trace for 78-nt ssDNA (Table 6) translocation through an [AuCl4]⁻¹ embedded MspA-M nanopore. The current trace was recorded at +100 mV with 4 μM 78-nt ssDNA and 10 μM HAuCl₄ in cis. Sequential and reversible [AuCl₄]- binding within an MspA nanopore finely modulates the pore cavity for ssDNA translocation. Level n (n=0-3) indicates n [AuCh]⁻ ions embedded in the pore restriction simultaneously. Enlarged blockage amplitudes and retarded dwell time for ssDNA translocation are observed from level n with larger n values. (b)

Scatter plot of absolute blocked depth vs dwell time and the corresponding current drop amplitude histograms for different numbered levels. (c, d) Mean 66 I/I₀ and dwell time for ssDNA translocation events from different level n. All means and standard deviations are from three independent experiments (10 minutes recording, N=3, Table 8). The image inset in d shows an expanded view for level 0-2.

FIG. 17 shows background current of M2 MspA with L-methionine addition. (a) A cartoon scheme of the M2 MspA. Site 91, which is yellow colored, represents where asparagine locates. (b) Electrophysiology recording using M2 MspA with L-methionine in cis reaching 1 mM final concentration. No direct sensing signal is observed at all in this configuration as no interaction could be formed between the analyte and the pore. (c) Electrophysiology recording using M2 MspA further HAuCl₄ to (b) reaching a 10 uM in final concentration. No specific recognition of L-methionine event was observed in (b) and (c) due to lack of sulfur-containing residues in the vicinity of the restriction site. The electrophysiology recordings above were carried out in 1.5 M KCl buffer with +100 mV applied potential (Methods).

FIG. 18 shows Specific recognition of L-methionine within the MspA-M with single Au(III) embedment. (a) Reversible and sequential binding of L-methionine with [AuCl4]⁻¹ embedded MspA-M. The current trace was recorded at +100 mV with 80 μM L-methionine io and 4 μM HAuCl₄ added in cis. Deep blockages reaching state 3 correspond to L-methionine binding. Four types (I-IV) of signals with identical amplitude are observed. (b) Four types of L-methionine blockage events as extracted from the trace in (a). Each event can be decomposed into various combinations of states 1, 2 and 3. (c) A molecular model for L-methionine binding with the methionine residue 91. State 3 represents the molecular is configuration of L-methionine binding with the pore restriction by taking Au(III) as an atomic bridge. (d) Scatter plot of ΔI₁₋₃ vs dwell time with the corresponding amplitude histogram. ΔI₁₋₃ stands for the amplitude difference between I and [M]-(AuCl₄⁻)-M. (Table 9).

FIG. 19 shows detailed interpretation of L-methionine sensing with MspA-M. (a) The mechanism for [AuCl_{4]}⁻¹ sensing in MspA-M . (b) When pre-mixed together, L-methionine and [AUCl₄]⁻ may form complexes spontaneously. (c) When embeded with Au(III) as an atomic bridge, MspA-M can bind freely translocating L-methionine. (d) The spontaneously formed molecular complex as demonstrated in (b) can interact with the methionine on the pore. Either reaction as demonstrated in c and d results in state 3 in FIG. 18c. For simplicity, [M] stands for the methionine residue introduced in MspA-M, M stands for freely translocating L-methionine and [Au] stands for dissociated tetrachloroaurate(III) ions.

FIG. 20 shows an expanded view of L-methionine binding event. When bridged with an Au(III) atom, L-methionine binding within the MspA-M results in a deep blockage with ˜48 pA current drop from I in either type of binding event (FIG. 18b). Different from the open pore level or the tetrachloroaurate(III) binding level, the L-methionine binding level systematically show violent baseline fluctuations with triangular noises. It is suspected that potential configuration switching of L-methionine when bound with MspA-M may have been observed. This fluctuation shape may be adopted as a signal characteristics for L-methionine recognition. Further investigations on this phenomenon may be carried out in a separate study.

FIG. 21 shows recording with L-asparagine and L-glycine using MspA-M. (a) A representative current trace recorded using MspA-M in 1.5 M KCl at +100 mV applied potential with 10 μM HAuChin cis. M—(AuCl₄⁻¹)ₙ binding levels with n=1-3 are clearly recognized (FIG. 8). (b) Electrophysiology recordings as demonstrated in (a) with further addition of L-asparagine to cis reaching a 1 mM final concentration. (c) Electrophysiology recordings as demonstrated in (a) with further addition of L-glycine to cis reaching a 1 inIVI final concentration. No extra sensing signals for L-asparagine and. L-glycine were observed in M2 MspA as demonstrated in (b) and (c). The negative sensing results as demonstrated with L-asparagine and L-glycine conclude that the sulfur containing side group of L-methionine is critical binding with the Au(III) bridge atom. This phenomenon could be double confirmed with previous demonstration of negative tetrachloroaurate(III) sensing with α-HL M113G and M2 MspA, where the amino acid residue on the pore restriction were glycine and asparagine, respectively.

FIG. 22 shows embedding different metal ion types within an engineered MspA nanopore. (A) N91M mutation of MspA for gold atom embedment. N stands for asparagine and M stands for methionine. Left: a top view of the N91M mutant of an MspA nanopore (MspA-M). Middle: the binding mechanism of methionine on the pore constriction with an AuCl₄⁻ ion. AuCl₄⁻ is the polyatomic form of a gold atom. Right: representative traces containing current blockade for single and multiple AuCl4⁻ ions. Here, [M]-(AuCl₄⁻)ₙ stands for different binding levels, where the footnote n stands for the number of AuCl₄⁻ ions io simultaneously in the pore restriction. (B) N91H mutation of MspA for (Zn²⁺, Cd²⁺, Co²⁺, Ni²⁺, Pb²⁺) embedment. Left: a top view of the N91H mutant of an MspA nanopore (MspA-H). N stands for asparagine and H stands for histidine. Middle: the binding mechanism of histidine with listed ions. Right: Representative current traces containing blockade events from binding of single Zn²⁺, Cd²⁺, Co²⁺, Ni²⁺, Pb²⁺ions respectively.. (C) N91C mutant of MspA for (Zn²⁺, Cd²⁺, Pb²⁺) embedment. N stands for asparagine and C stands for cysteine. Left: a top view of the N91C mutant of an MspA nanopore (MspA-C). Middle: the binding mechanism of cysteine with listed metal ions. Right: representative current blockade events for from binding of Zn²⁺, Cd²⁺, Pb²⁺ ions, respectively. The mutation of N91M, N91H or N91C is in reference to M2 MspA.

FIG. 23 shows engineered MspA nanopore at various locations for AuCl₄⁻ embedment. (A) The schematic diagram of the site directed mutagenesis locations. The amino acid at position 88, 91 or 105 is mutated to methionine. The image inset on the right represents the mutation locations within a chain of the monomeric peptide chain near the nanopore constriction. (B) Representative current blockade from AuCl₄⁻ embedment at position 91. (C) Representative current blockade from AuCl₄⁻ embedment at position 88. (D) Representative current blockade from AuCl₄⁻ embedment at position 105. In principle, every amino acids within an MspA nanopore could be engineered for metal embedment. Due to the conical geometry of the MspA nanopore, site 83 to site 111, which forms the pore constriction are preferred engineering sites.

FIG. 24 shows MspA-M and biothiols. The background current was recorded with MspA-M when a +100 mV voltage was continuously applied (Methods). No HAuCl₄ was added in cis for this set of measurements. (a) The background current recorded using MspA-M without the addition of any biothiols. MspA-M stays open with no spontaneous gating activities in this condition. (b-d) The background current recorded using MspA-M with 40 μM Cys (b), 40 μM Hcy (c) or 40 μM GSH (d) in trans. No Cys, Hcy or GSH binding event were observed. Without the Au(III) embedment as an atomic bridge, single molecule sensing of biothiols cannot be directly performed with MspA-M.

FIG. 25 shows stochastic sensing of L-cysteine by Au(III) embedded MspA-M. (a) A general schematic diagram of biothiols sensing by Au(III) embedded MspA-M. A single MspA-M nanopore was inserted in a lipid bilayer separating the cis and trans compartments of a nanopore system (Methods). HAuCl₄ were added in cis while the biothiols (R-SH) were added in trans. Here R represents the chemical structure of a specific type of biothiol other than the thiol group. (b) A molecular model for biothiols sensing with Au(III) embedded MspA-M. State 0 or 1 represents a methioine residue, which is around the pore restriction (N91M), without or with a [AuCl4]⁻¹ embedment. State 1_{S}H represents the molecular configuration of a biothiol bound with the pore restriction by taking the embedded Au(III) as an atomic bridge. (c) The chemical structure of L-cysteine (Cys). L-cysteine is a specific type of biothiol. (d) Reversible and sequential binding of Cys with [AuCl4]⁻¹ embedded MspA-M. The demonstrated trace was recorded with a +100 mV applied voltage when 4 μM of HAuCl₄ in cis and 40 μM of Cys in trans were present simultaneously. Resistive pulse signals in the trace result from binding of [AuCl4]⁻¹ or binding of a Cys by taking the bound Au(III) as an atomic bridge. Red triangles mark the events from Cys. The grey star marks the event of [AuCl₄]⁻. Other unlabeled events are background signals as described in FIG. 26. (e) A io representative Cys blockage event. From this zoomed-in demonstration, a representative Cys blockage event is composed of three states that correlate with the molecular model in b. State 1_{SH} appears as a characteristic jitter signal. Higher grey marked region or lower pink marked region represent the area in which the transition of state 0-1 or state 1-1_{SH} distributes, respectively. (f) The scatter plot of the relative blockade amplitude ΔI/I₀ vs the dwell time is from events of [AuCl_{4]}⁻¹ and Cys binding. The event amplitude (Al) for [AuCl_{4]}⁻¹ or Cys is defined as the current difference between state 1 or state 1_{SH} in reference to state 0 (FIG. 27). I is the open pore current, was derived from the mean value of state 0. (g) The histogram of ΔI/I₀ for single [AuCl₄]⁻ bindings and Cys bindings. (h) The histogram plot of the dwell time of state 1_{S}H (t_{off,cys}) from a series of Cys binding events. The mean dwell time τ_{off,cys} was derived from the single exponential fitting result. The statistical data in (f), (g) and (h) were from a continuous 10 min recording (Methods). (Tables 10, 11).

FIG. 26 shows signal types of biothiols sensing. (a) The background current recorded using MspA-M without any analyte addition. Though no spontaneous gating was observed from MspA-M, during a long term of measurement with a +100 mV continuously applied voltage, transient pore blockages could still be observed. However, the blockage depth of these transient events is widely distributed. (b) A representative current trace recorded with MspA-M when a +100 mV voltage was continuously applied. 4 μM HAuCl₄ in cis and 40 μM Cys in trans were placed. The events of [AuCl4]⁻¹ or Cys binding show a narrow distribution of the blockage amplitude and could be easily discriminated from the background events. (c) A zoomed-in view of all possible event types during the measurement as described in b. Type 1 is a representative [AuCl_{4]}⁻¹ binding event, as judged by its shape and blockage amplitude (FIG. 6b). Type 1 event is excluded from the statistics of biothiols sensing as clearly no biothiol has bound with the pore when this event was acquired. Type 2 event is a representative biothiol event as discussed in FIG. 25e and is included in the statistics of biothiol sensing. Type 3 event is also a representative biothiol event. Transient spiky signals on top of the [AuCl_{4]}⁻¹ binding state may be occasionally observed. Type 3 events were also included in the statistics. Type 4 and 5 events contains simultaneous binding of more than 1 [AuCl₄]⁻ in the pore, as judged from the shape and the event blockage depth (FIG. 8). To avoid complicating the statistics, these events were excluded from the statistics. Type 6 is the background event as observed in a. Type 6 events were excluded from the statistics.

FIG. 27 shows data analysis. All nanopore events ([AuCl₄]⁻, Cys, Hcy or GSH) were extracted by the single-channel search feature of ClampFit, if not otherwise stated. The extraction result is demonstrated in this figure by taking a continuous trace of Cys sensing as an example (FIG. 25d). The mean value of state 0, 1, 1_{S}H were marked with large interval dotted line, small interval dotted line and solid line respectively. The definition of the event dwell time t_{off,|Au|} and t_{off,Cys} were marked on the figure. ΔI₀₋₁ stands for the amplitude difference between I₀ and I₁. ΔI_{1-1,Cys} stands for the amplitude difference between I₀ and I_{1,Cys}.

FIG. 28 shows the dwell time of state 1 after the addition of Cys. (a) A representative trace for single [AuCl4]⁻¹ binding with MspA-M when 4 μM HAuCl₄ were placed in cis. Grey stars mark the events of [AuCl4]⁻¹ binding. (b) A representative trace for [AuCl4]⁻¹ binding with MspA-M when 4 μM HAuCl₄ in cis and 40 μM Cys in trans were placed. Red triangles mark the signals of Cys. Whereas, the grey star marks the [AuCl_{4]}⁻¹ binding event. Other unlabeled events are background signals (FIG. 26). (c) The dwell time (t_{off}) histogram of single [AuCl₄]⁻¹ binding before and after the addition of 40 μM Cys in trans. Due to the competitive binding of Au(III) with the thiol of the cysteine against the thioether of the methionine around the pore restriction, the dwell time of state 1 is greatly shortened. (d) The bar plot of the mean dwell time (τ_{off}) of single [AuCl₄]⁻¹ binding before and after the addition of 40 μM Cys in is trans. All means and standard deviations were from three independent experiments. (Before: 530±90 ms; After: 38±3 ms)

FIG. 29 shows MspA-M and amino acids other than biothiols. Electrophysiology recordings were performed with MspA-M when a +100 mV voltage was continuously applied and 10 μM HAuCl₄ was added in cis. (a) A representative current trace without the addition of any amino acids. Sequential binding of three [AuCl₄]⁻¹ were clearly recognized, as described in FIG. 11. A representative current trace when recorded with a further addition of L-asparagine (b), L-glycine (c), L-glutamic acid (d) to cis reaching a 1 mM final concentration. As demonstrated in b-d, no clear sensing events, as observed from L-cysteine (FIG. 25e), were observed for L-asparagine, L-glycine and L-glutamic acid. This indicates that a thiol group is critical in the generation of the event as demonstrated in FIG. 25e.

FIG. 30 shows stochastic sensing of L-homocysteine by Au(III) embedded MspA-M. (a) The chemical structure of L-homocysteine (Hcy), a homologue of Cys with an extra carbon in its structure (green shading labeled portion). (b) Reversible and sequential binding of Hcy with [AuCl₄]⁻¹ embedded MspA-M. The demonstrated trace was recorded with a +100 mV applied voltage when 4 μM HAuCl₄ in cis and 40 μM Hcy in trans were present simultaneously. Resistive pulse signals in the trace result from binding of [AuCl4]⁻¹ or binding of a Hcy by taking the bound Au(III) as an atomic bridge. Green diamonds mark the events of Hcy. The grey star marks the events of [AuCl₄]⁻. Other unlabeled events are background signals as described in FIG. 26. (c) A representative Hcy blockage event. Similar to FIG. 25E, from this zoomed-in demonstration, a representative Hcy blockage event is composed of three states that correlate with the molecular model in FIG. 25b. Characteristic jitter signals can be recognized as state 1 _{S}H. However, the mean blockage amplitude of state 1 _{S}H for Hcy is systematically deeper than that of Cys. Higher grey marked regions and lower green marked regions represent the area in which the transitions of state 0-1 or state 1-1 _{S}H are distributed, respectively. (d) The scatter plot of the ΔI/I₀ vs the dwell time from events of [AuCl₄]⁻¹ and

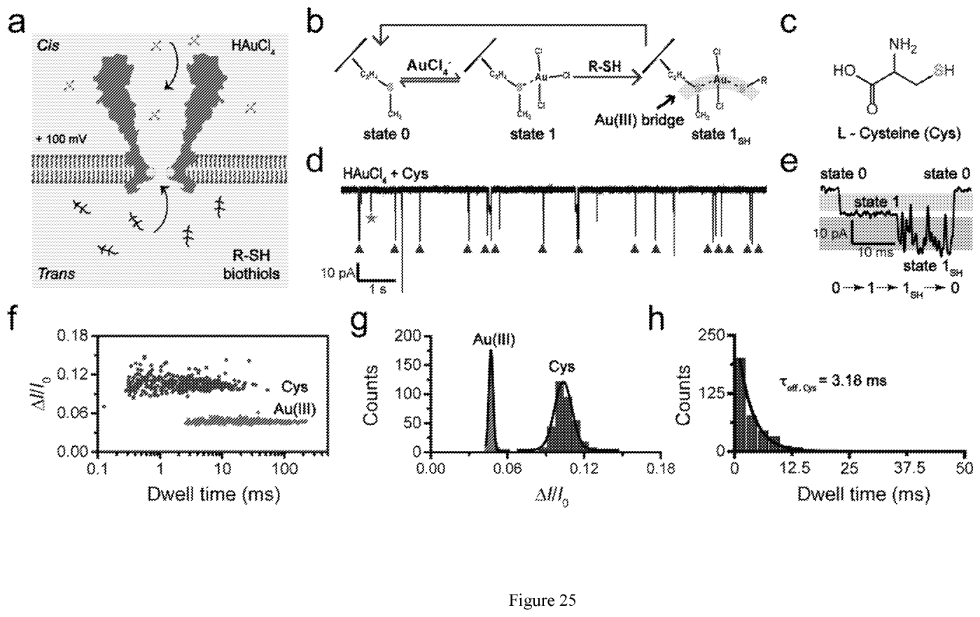

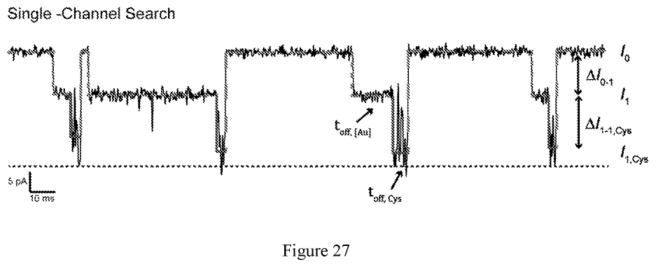

Hcy binding. The definition of ΔI and I₀ is the same as that described in FIG. 25f. (e) The histogram of ΔI/I₀ for single [AuCl₄]⁻ bindings and Hcy bindings. The statistical data in (d) and (e) were derived from a continuous 10 min recording with a +100 mV applied voltage (Table 12, 13). (f) Simultaneous discrimination of Hcy and Cys with [AuCl₄]⁻¹ embedded MspA-M. The demonstrated trace was recorded with a +100 mV applied voltage when 4 μM HAuCl₄ in cis were present. Cys and Hcy were placed in trans with a concentration of 20 μM for each analyte. Red triangles mark the events of Cys while green diamonds mark those of Hcy. The grey star marks the event of [AuCl₄]⁻. A zoomed-in view of events from Hcy or Cys were demonstrated in the dotted box. (g) The scatter plot of the 61//₀ vs the dwell time with the corresponding amplitude histogram from Hcy and Cys sensing. (FIG. 27) The plot data were from a continuous 10 min recording as described in f.

FIG. 31 shows dwell time analysis. The statistical data were from electrophysiology recordings with MspA-M when 4 μM HAuCl₄ in cis and 40 μM Cys, Hcy or GSH in trans io were present. (a) Representative blockage events of Cys, Hcy and GSH. The dwell time were marked on the event respectively. (b) Histograms of the [AuCl₄]⁻¹ binding dwell time (t_{off},ᵢ₋₃) when 40 μM Cys (t_{off,1}), Hcy (t_{off,2}) or GSH (t_{off,3}) were placed in trans respectively, as marked differently in the figure. Here t_{off,1-3} stands for the dwell time of state 1 (FIG. 25e). The mean dwell time (τ_{off,1-3})was derived from single exponential fitting results (FIG. 32). (c) Histograms of the biothiols binding dwell time for Cys(t_{off,Cys}), Hcy (t_{off,Hcy}) or GSH (t_{off,GSH}). Here, t_{off,Cys}, t_{off,Hcy} or t_{off,GsH} stands for the dwell time of state 1_{SH} (FIG. 25e). The mean dwell time τ_{off} were derived from single exponential fitting results (FIG. 32). (d) Mean dwell time of [AuCl₄]⁻¹ binding events. The HAuCl₄ concentration in cis were kept with 4 μM. τ_{off}, τ_{off,1}, τ_{off,2} or τ_{off,3} stands for the dwell time of the [AuCl₄]⁻¹ binding state when no biothiol, Cys, Hcy or GSH were added respectively. The binding of thiol-containing amino acids or peptides has significantly shortened the dwell time of [AuCl₄]⁻ bound with the methionine around the pore restriction. This effect is most pronounced for Cys, followed by Hcy and GSH. All means and standard deviations were from three independent experiments for each condition (Table 10, 12, 14). (e) The mean dwell time of Cys, Hcy and GSH bound in the pore. All means and standard deviations were from three independent experiments (10 min recording, N=3, Table 10, 12, 14)

FIG. 32 shows analysis of the dwell time and the inter-event intervals. (a) A representative electrophysiology trace of tetrachloroaurate(III) binding with a MspA-M nanopore. tₒₙ represents the inter-event duration time. Whereas, t_{off} represents the event dwell time. (b-c) Histogram plots of the inter-event duration time (tₒₙ) and the event dwell time (t_{off}) with corresponding single exponential fittings respectively. The single exponential fitting was performed according to the equation y=a*exp (−x/T). The mean inter-event interval (τₒₙ) or the mean dwell time (τ_{off}) was derived from the fitting result respectively. All mean dwell time and inter-event intervals values in this invention were derived as described if not otherwise stated.

FIG. 33 shows stochastic sensing of L-Glutathione by Au(III) embedded MspA-M. (a) The chemical structure of L-Glutathione (GSH). GSH is a tripeptide containing a cysteine residue (blue shading labeled portion). (b) Reversible and sequential binding of GSH with [AuCl4]⁻¹ embedded MspA-M. The demonstrated trace was recorded with a +100 mV applied voltage when 4 μM HAuCl₄ in cis and 40 μM GSH in trans were present simultaneously.

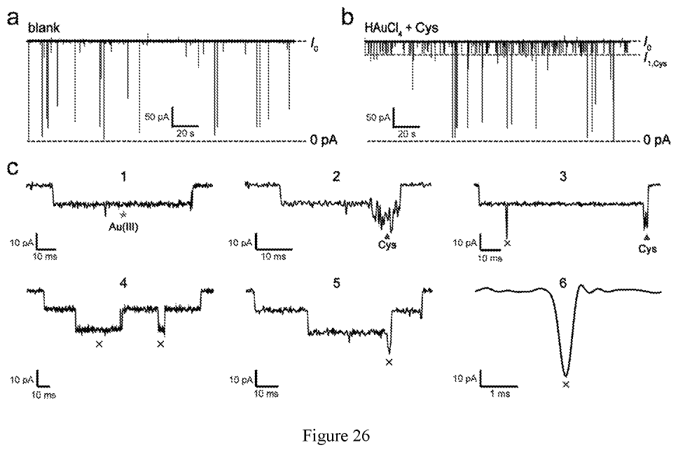

Resistive pulse signals in the trace represent GSH binding with the pore. Blue dots mark the events concerning GSH. Other unlabeled events are background signals as described in FIG. 26. (c) A representative GSH blockage event. Characteristic jitter signals similar to that of Cys or Hcy can be recognized as state 1_{SH}. However, the mean blockage amplitude of state 1 _{SH} for GSH is systematically deeper than that of Cys or Hcy. Grey and blue marked regions represent the the area that the transition of state 0-1 or state 1-1 _{SH} distributes respectively. (d) The scatter plot of ≢6I/I₀ vs the dwell time with the corresponding amplitude histogram from events of [AuCl₄]⁻ or GSH binding. The definition of ΔI and I₀ is the same as that described in FIG. 25f. The statistical data in (d) were from a continuous 10 min recording as described in b. (Table 14, 15). (e) Simultaneous discrimination of GSH and Cys with [AuCl₄]⁻¹ embedded MspA-M. Cys and GSH were placed in trans with a concentration of 10 μM and 30 μM respectively. Whereas, the concentration of HAuCl₄ in cis was kept at 4 μM. In the dotted box, io a zoomed-in view of GSH and Cys events were demonstrated. Red triangles mark the events of

Cys and blue dots mark those of GSH. The grey star marks binding events from one or two [AuCl₄]⁻. (f) The scatter plot of the dwell time vs the ΔI/I₀ with the corresponding amplitude histogram from the simultaneous recording of Cys and GSH as a mixture with 10 μM and 30 μM in trans respectively. The statistical data were derived from a continuous 10 min recording.

FIG. 34 shows discrimination of L-cysteine, L-homocysteine and L-glutathione by Au(III) embedded MspA-M. (a) Representative binding events of Cys, Hcy or GSH acquired with Au(III) embedded MspA-M nanopore. (b) The histogram of ΔI/I₀ with corresponding Gaussian fittings from events of Cys, Hcy and GSH. (N=330 for each distribution) (c) The bar plot of the mean ΔI/I₀ values of Cys, Hcy and GSH. (Cys: 0.104±0.0006, Hcy: 0.128±0.004, GSH: 0.154±0.011). All means and standard deviations were from three independent experiments for each analyte (10 min continuous recordings, Table 11, 13, 15).

### DETAILED DESCRIPTION

This invention demonstrates that wild-type (WT) α-HL is a natural [AuCl₄]⁻¹ sensor as a result of the coordination of Au[III] with methionine(113). This sensing mechanism can be transplanted to the MspA nanopore[22, 23] with a significantly amplified event amplitude, up to ˜54.88 pA. MspA with Au(III) embedment continues to permit ssDNA (e.g. Table 6) translocation by geometric modulation of the pore restriction with atomic accuracy. Highly specific recognition of L-methionine by MspA is also demonstrated in assistance of Au(III) embedment, which may inspire a new approach for nanopore based protein sequencing. To the best of our knowledge, single ion interactions of Au(III) with a biological nanopore have never been investigated. Binding of tetrachloroaurate(III) within an engineered MspA nanopore is also the largest event amplitude reported from a single ion. Studies of this first hybrid biological pore embedded with Au(III) species may further inspire novel applications by merging the sensing features of biological nanopores and gold to produce a “gold biological nanopore”.

A protein nanopore can be used to detect single molecule. Some proteins can self-assemble in a lipid bilayer membrane to form a nanopore with a vestibule and a limiting aperture. The limiting aperture of the nanopore allows single molecules such as single ion or single-stranded nucleic acid molecule to pass through. In an aqueous ionic salt solution such as KCl, when an appropriate voltage is applied across the membrane, the pore formed by the nanopore channel conducts a sufficiently strong and steady ionic current. The single molecule is driven through the pore by the applied electric field, thus blocking or reducing the ionic current which can be detected. The duration of the blockade and the signal strength is related to the identity of the single molecule, such as the identity of metal-containing ion or the four bases (A, C, G and T) composition of a nucleic acid. The duration of the blockade and the signal strength also can be related to the identity of the single molecule, such as the identity of any amino acid such as L-methionine.

In the invention, the term “nanopore” refers to a pore having an opening at its narrowest point having a diameter when molecule of interest pass through the opening, the passage of the molecule can be detected by a change in signal, for example, electrical signal, e.g. current. In some cases, the nanopore is formed by protein within a membrane which may be referred to protein nanopore. Examples of protein nanopore or protein which can form a nanopore include io alpha-hemolysin, MspA, CsgG, OmpG, Cytolysin A, ClyA, aerolysin, Frac or Phi29 connector. The protein nanopore can be modified or unmodified. The protein nanopore can be modified by mutation in one or more amino acids. In some embodiments, the protein nanopore may be mutated in one or more amino acids on the inner surface. Generally, protein nanopore has vestibule and constriction zone. In some cases, the nanopore is disposed within a membrane, or is lipid bilayer. In some embodiments, the protein has a conically shaped passage which acts as a conically shaped biological nanopore.

In the invention, the protein used preferably can insert spontaneously into the membrane to form a nanopore. The protein nanopore used in this invention preferably has no spontaneous gating activities at positive voltages (up to +200 mV) and/or preferably keeps open at positive applied voltages with open pore conductance. The protein nanopore used in this invention preferably may have one or more amino acid residues which can interact with the metal ion on the inner surface of the nanopore channel. The protein nanopore may be modified to have one or more amino acid residues which can interact with the metal ion on the inner surface. One or more amino acid residues on the inner surface of the protein nanopore may be mutated to amino acid residues which can interact with the metal ion, such as methionine, cysteine or histidine. In some embodiments, the protein nanopore may have methionine, cysteine or histidine on the inner surface.

In the invention, the term “α-hemolysin”, is also referred to as a-HL, may be selected from the group consisting of a wild-type α-hemolysin, a mutant a-hemolysin, a wild-type α-hemolysin paralog or homolog hemolysin, and a mutant a-hemolysin paralog or homolog hemolysin. In some embodiments, a-hemolysin may be the wild-type a-hemolysin. The α-hemolysin that may be used in the invention should be capable of forming nanopore.

In the invention, the term “MspA”, “MspA porin” and “MspA nanopore”can be used interchangebly and refers to Mycobacterium smegmatis porin A (MspA). As known by the person skilled in the art, a MspA porin can comprise two or more MspA monomers (e.g., eight monomers), which associate with each other and form a tunnel, wherein each monomer may be the same of different. MspA may be an octameric MspA. The MspA porin that may be used in the invention should be capable of forming nanopore. Any one MspA monomer that formed the MspA porin may be selected from the group consisting of a wild-type MspA monomer, a mutant MspA monomer, a wild-type MspA paralog or homolog monomer, or a mutant MspA paralog or homolog monomer. In some embodiments, all monomers in a MspA porin are the same, such as the same mutant MspA monomers.

In this invention, the term “mutant MspA” refers to a mutant of wild type MspA. Wild type MspA is comprised of wild type MspA monomers. Mutant MspA may comprises at least one mutant MspA monomers, and the remaining monomers in the mutant MspA may be selected from the group consisting of a wild-type MspA monomer, a mutant MspA monomer, a wild-type MspA paralog or homolog monomer, or a mutant MspA paralog or homolog monomer. Mutant MspA may comprise two or more MspA monomers (e.g., eight monomers) and mutant MspA may be an octameric MspA.

In some embodiments, in a mutant MspA porin, one or more monomers are mutant MspA monomers and the other monomers are wild-type MspA monomers. In some embodiments, the MspA porin is comprised of eight mutant MspA-M monomer. When a MspA comprises more than one mutant MspA monomers, said more than on mutant MspA monomers may be the io same or different.

MspA porin may be mutated for metal embedment. In principle, every amino acids within an MspA nanopore could be engineered for metal embedment. Due to the conical geometry of the MspA nanopore, site 83 to site 111, which forms the pore constriction are preferred engineering sites. Therefore, introduction of amino acid(s) suitable for metal embedment, such as methionine, cysteine or histidine, at any one or more residue of site 83 to site 111 would significantly amplify binding signals around the pore restriction at these residues. Residue 91 is the narrowest spot of MspA, introduction of amino acid(s) suitable for metal embedment at residue 91 would result in an excellent signal amplification. Mutation at residue 88 to 105 would render similar effect with residue 91. In some embodiments, the mutant MspA monomer may comprise one or more mutations at positions 83-111 of MspA. In some embodiments, at least one of the mutant MspA monomers (e.g., all of eight mutant MspA monomers) in a mutant MspA may comprise one or more mutations at position 83-111. In some embodiments, the one or more mutations at positions 83-111 may be one or more mutations at position 83, 84, 85, 86, 87, 88, 89, 90, 91, 92, 93, 94, 95, 96, 97, 98, 99, 100, 101, 102, 103, 104, 105, 106, 107, 108, 109, 110 and/or 111. In some embodiments, the one or more mutations at positions 83-111 may be one or more mutations at position 88, 89, 90, 91, 92, 93, 94, 95, 96, 97, 98, 99, 100, 101, 102, 103, 104 and/or 105. In some embodiments, the one or more mutations at positions 83-111 may be one or more mutations at position 88, 91 and/or 105. In some embodiments, the mutation at any position of site 83-111 may be independently the mutation from the natural residue to the amino acid suitable for metal embedment, such as methionine, cysteine or histidine. In addition to the mutation(s) at position 83-111, the mutant MspA monomer can also comprise mutation(s) at any other positions. The mutant MspA monomer may only has the mutation(s) at position 83-111 compared to the wild-type MspA monomer. The one or more mutations at position 83-111 may be mutation to methionine, cysteine, histidine or any combination of them.

In some embodiments, the mutant MspA monomer may be a mutant MspA monomer which comprises a mutation of D91M, D91H or D91C. In some embodiments, at least one of the mutant MspA monomers (e.g., all of eight mutant MspA monomers) in a mutant MspA may comprise a mutation of D91M, D91H or D91C. In some embodiments, the mutant MspA monomer may be the mutant MspA-M monomer which comprises the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer. In some embodiments, at least one of the mutant MspA monomers (e.g., all of eight mutant MspA monomers) in a mutant MspA may comprise the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer. In some embodiments, the mutant MspA monomer may be the mutant MspA-M monomer which only has the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer. In some embodiments, at least one of the mutant MspA monomers (e.g., all of eight mutant MspA monomers) in a mutant MspA may only has the mutations of D93N/D91M/D9ON/D118R/D134R/E139K, D93N/D91H/D9ON/D118R/D134R/E139K, or D93N/D91C/D9ON/D118R/D134R/E139K compared to the wild-type MspA monomer. D93N/D91M/D9ON/D118R/D134R/E139K, D93N/D91H/D9ON/D118R/D134R/E139K, or D93N/D91C/D9ON/D118R/D134R/E139K means that the mutant comprises simultaneously all of these six mutations. The number used here identifies the location of site directed mutagenesis, where the first amino acid immediately after the start codon is defined as 1.

Sequences of wild type MspA monomers are known by the person skilled in the art. For example, Sequences of wild type MspA monomers can be found in GenBank on https://www.ncbi.nlm.nih.gov/. In some embodiments, the wild-type MspA porin monomer may have the following amino acid sequence:

(SEQ ID NO: 1)
GLDNELSLVDGQDRTLTVQQWDTFLNGVFPLDRNRLTREWFHSGRAKYI
VAGPGADEFEGTLELGYQIGFPWSLGVGINFSYTTPNILIDDGDITAPP
FGLNSVITPNLFPGVSISADLGNGPGIQEVATFSVDVSGAEGGVAVSNA
HGTVTGAAGGVLLRPFARLIASTGDSVTTYGEPWNMN.

In some embodiments, the wild-type MspA porin monomer may be consisted of SEQ ID NO: 1.

The preparation method of a-hemolysin or MspA is known by the person skilled in the art, for example, it could be prepared by prokaryote expression and easily purified by chromatography.

In one aspect of this invention, the inventors have found that MspA can sense tetrachloroaurate(III) ions with more amplified resistive pulses (up to 54.88 pA) due to the focusing geometry of MspA. Therefore, it is believed that MspA nanopore can be used as a good metal-containing ion sensor.

Ion that can be identified by MspA nanopore may be any metal-containing ion. Said metal-containing ion may include metal ion and complex ion formed by metal and other ion. In some embodiments, the metal-containing ion that can be identified by MspA nanopore may contains Au(III), i.e. trivalent gold ion, such as Au'. In some embodiments, the metal-containing ion that can be identified by MspA nanopore may be tetrachloroaurate(III) ion. In some embodiments, the metal-containing ion that can be identified by MspA nanopore may include Zn²⁺, Co²⁺, Ag⁺, Cd²⁺, Ni²⁺, Au(I) such as [AuCl₂]⁻, Cu²⁺, Fe²⁺, Pt(II), Pd(II), Mn²⁺, Hg²⁺, R(II) or Pb²⁺.

In another aspect of this invention, the inventors have found that tetrachloroaurate(III) embedded MspA nanopores are capable of translocating [AuCl₄]⁻¹ or ssDNA with enlarged blockage amplitude and enhanced capture rate due to a channel cavity which has been finely tuned by metal-containing ion embedding. Therefore, it is believed that protein nanopore embedded with metal can be used to identify an analyte with better sensitivity.

In this invention, the protein nanopore may be a protein nanopore embedded with metal. Metal adhesion on the inner surface of the protein nanopore narrows the channel of the nanopore and amplifies the signal change when the analyte translocates through the nanopore. Said metal can interact with one or more amino acid residues (such as methionine, cysteine and/or histidine) on the inner surface of the nanopore channel. The metal used in this invention may be the metal that can interact with any one of the amino acid residues (such as methionine, cysteine and/or histidine) on the inner surface of the nanopore channel.

In principle, any metal-amino acid interaction may be used for the method of the present invention. If a metal is capable of interacting with an amino acid, the ion containing said metal can be used to modify the nanopore that contain said amino acid on the inner surface to form the metal embedded protein nanopore of the present invention. It should be understood that many metal-containing ions and many protein nanopores can be used and are not limited to the examples illustrated in the present invention, providing that the metal-containing ions are capable of interacting with the amino acid on the inner surface of the nanopore. It has been known that many metal ions are capable of interacting with the group of the amino acid or with the structure formed by several amino acids, such as transition metal ions are easy to coordinates with the amino acids. The metal-containing ions used in the present invention include, but is not limited to ions containing transition metal, such as transition metal ions. Metal ions' coordination to specific groups may be predicted, for example by the theory of HSAB which was first proposed by Pearson in 1963[64] and its principle is that “hard acids prefer to coordinate to hard bases, and soft acids to soft bases”. HSAB theory is mainly applied to give a qualitative prediction or interpretation for the coordination results. Metal ions' coordination to specific groups may also be proved by an experiment of interaction.

In the invention, the term “interact with” refers to that the metal may bind to any amino acid residue or any structure formed by the amino acids on the inner surface of the protein nanopore in any way, for example, in a reversible way or in an irreversible way.

The metal that is embedded on the inner surface of the nanopore may be in the form of metal-containing ion and may be any suitable metal-containing ion. Said metal-containing ion include metal ion and complex ion formed by metal and other ions. The type of metal-contain ions which are bound to the amino acid residue on the inner surface of the protein nanopore may be one or more. The number of metal-contain ions which are bound to the amino acid residue on the inner surface of the protein nanopore may be one or more, e.g. 1, 2, 3, 4, 5, 6, 7, or 8. The inventor has found that a great number of metal-containing ions bound to the inner surface of the nanopore will result in larger pore blockage amplitudes and more significant amplification of the signal change when the analyte translocates through the nanopore.

Many metal-containing ions can be bound to the amino acid of the inner surface of the nanopore and narrow the pore restriction, thereby amplify the pore blockage amplitude due to an increased electric field around the sensing spot where the analyte binds. In some embodiments, the metal-containing ions that are bound to the inner surface of the nanopore may contain Au(III), such as Au'. In some embodiments, the metal-containing ions that are bound to the inner surface of the nanopore may be tetrachloroaurate(III) ion (that is [AuCl₄]⁻). [AuCl₄]⁻¹ ion may be bound to methionine and /or cysteine on the inner surface of the nanopore. In some embodiments, the number of [AuCl₄]⁻¹ ion molecules which are bound to methionine and/or cysteine on the inner surface of the nanopore is 1, 2, 3, 4, 5, 6, 7, or 8. Based on similar principle, in some embodiments, the metal-containing ions that are bound to the inner surface of the nanopore may contain or be Zn²⁺, Co²⁺, Ag⁺, Cd²⁺, Ni²⁺, Au(I) such as [AuCl₂]⁻, Cu²⁺, Cr³⁺, Fe²⁺, Fe³⁺, Pt(II), Pd(II), Mn²⁺, Hg²⁺, Ru(II) or Pb²⁺. Zn²⁺, Cd²⁺, Co²⁺, Ni²⁺, or Pb²⁺ ion may be bound to histidine on the inner surface of the nanopore. Zn²⁺, Cd²⁺, or Pb²⁺ ion may be bound to cysteine on the inner surface of the nanopore. In some embodiments, one or more metal-containing ions may be bound to more than one amino acid residue on the inner surface of the protein nanopore to enhance the amplification effect. Therefore, the protein nanopore may have more than one amino acid residue on the inner surface that can interact with the metal-containing ions. The metal-containing ions bound to the same site may be the same or different. The metal-containing ions bound to different site may be the same or different.

The protein nanopore embedded with metal and the method of this invention can be used to detect analyte in a single molecule. In this invention, said analyte may be capable of passing through a nanopore channel in a single molecule under an electric field and causing a change in current through the nanopore.

In any embodiment herein, the analyte may be a nucleotide, a nucleic acid, an amino acid, a peptide, a protein, a polymer, a drug, an ion, a pollutant, a nanoscopic object, or a biological warfare agent. In some embodiments, the analyte may be metal-containing analyte, such as metal-containing ion. Said metal-containing ion may include metal ion and complex ion formed by metal and other ions. In some embodiments, the metal-containing ion that can be identified by the protein nanopore embedded with metal may contains Au(III) such as Au³⁺. In some embodiments, the metal-containing ion that can be identified by the protein nanopore embedded with metal may be tetrachloroaurate(III) ion. In some embodiments, the metal-containing ion that can be identified by the protein nanopore embedded with metal may contains Zn²⁺, Co²⁺, Ag⁺, Cd²⁺, Ni²⁺, Au(I) such as [AuCl₂]⁻, Cu²⁺, Cr³⁺, Fe²⁺, Fe³⁺, Pt(II), Pd(II), Mn²⁺, Hg²⁺, Ru(II) or Pb²⁺. In some embodiments, the analyte is a polymer, such as a protein, a peptide, or a nucleic acid. Optionally, the polymer is a nucleic acid. The nucleic acid may be ssDNA, dsDNA, RNA, or a combination thereof. Optionally, the polymer is a peptide or a protein.

According to the invention, the metal embedded in the nanopore may be a metal-containing ion. The analyte to be detected may be a metal-containing analyte, such as a metal-containing ion. The metal embedded in the nanopore may be the same with or be different from the metal to be detected. For example, the metal embedded in the nanopore and the metal to be detected may be independently selected from the group consisting of Au(III), Zn²⁺, Co²⁺, Ag⁺, Cd²⁺, Ni²⁺, Au(I), Cu²⁺, Cr³⁺, Fe³⁺, Fe³⁺, Pt(II), Pd(II), Mn²⁺, Hg²⁺, Ru(II) and Pb²⁺, e.g. [AuCl₄]⁻¹ or [AuCl₂]⁻ ion.

When the analyte is nucleic acid such as ssDNA, different nucleotides may cause different current changes when passing through the nanopore, which enables sequencing of the nucleic acid. Thus, the protein nanopore embedded with metal and the method of this invention can be used to sequence a nucleic acid such as ssDNA. In some embodiments, the ssDNA can be in any length, for example, 1 nucleotide or more in length, 2 nucleotides or more in length, 3 nucleotides or more in length, 4 nucleotides or more in length, 5 nucleotides or more in length, 10 nucleotides or more in length, 20 nucleotides or more in length, 30 nucleotides or more in length, 40 nucleotides or more in length, 50 nucleotides or more in length, 70 nucleotides or more in length, 100 nucleotides or more in length. In some embodiments, the ssDNA is short oligomeric nucleic acid, such as miRNA, siRNA or short DNA probe.

The analyte to be detected may be an amino acid, such as an amino acid containing sulfur. The amino acid may be natural or non-natural. The amino acid may contain natural basic group or non-natural basic group. The amino acid may be selected from 20 kinds of amino acids that make up proteins or from other kinds of amino acids. In some embodiments, the analyte to be detected may be an amino acid having one or more sulfur atoms on the side chain. In some embodiments, the analyte to be detected may be an amino acid having —SH group. In some embodiment, the analyte to be detected may be L-methionine, L-cysteine, L-homocysteine or any other amino acids.

The analyte to be detected may be a peptide or a protein. When the analyte is a peptide or a protein, different amino acids may cause different current changes when passing through the nanopore, which enables sequencing of the peptide or protein. Thus, the protein nanopore embedded with metal and the method of this invention can be used to sequence a peptide or a protein. In some embodiments, the analyte to be detected may be a peptide or a protein containing sulfur. In some embodiments, the analyte to be detected may be a peptide or a protein containing the amino acid having one or more sulfur atoms on the side chain or the amino acid having -SH group. The analyte to be detected may be a peptide or a protein containing L-methionine, L-cysteine, L-homocysteine or any other amino acids.

Based strong interaction between Au(III)-thiol, the analyte to be detected may be a thiol. The term “thiol” refers to any molecule that includes one or more terminal -SH group. In some embodiments, the analyte may be a biothiol, which is any thiol that is commonly found in biological systems. Examples of biothiol include amino acids or peptides containing thiol group (-SH), exemplified by cysteine, homocysteine, and glutathione, etc.; several types of antioxidants (such as N-acetylcysteine), and several types of vitamins (such as thiamine).

In some embodiments, the analyte to be detected may be a peptide or a protein containing biothiols or thiols.

The method, the system and the kit of the present invention can be used to discrimination between different analytes, such as different ssDNA, different biothiols or peptides containing different biothiol or thiol, etc.

In this invention, the term “identifying” includes detecting or analyzing the type or the composition of the analyte. For example, the protein nanopore embedded with metal and the method of this invention can be used to detect a metal-containing analyte or analyze the nucleotide composition of a nucleic acid (A, C, G and T). Also, the protein nanopore embedded with metal and the method of this invention can be used to detect the type of the amino acid or analyze the amino acid composition of a peptide or a protein (each amino acid).

This invention also provides systems and methods of identifying an analyte in a sample using protein nanopore. A system and a method of identifying a metal-containing ion in a sample using MspA nanopore is provided when the protein nanopore is MspA nanopore and the analyte is a metal-containing ion. Systems and methods of identifying an analyte in a sample using protein nanopore embedded with metal when the protein nanopore is embedded with metal and the analyte is any analyte discussed herein.

The process of identifying an analyte using protein nanopore is known by the person skilled in the art, which can be used in this invention. Currently known and commonly used methods include positioning a membrane comprising a protein nanopore between a first conductive liquid medium and a second conductive liquid medium, wherein the nanopore comprises an opening that provides liquid communication, applying an electric field across the nanopore and translocating the analyte through the nanopore, measuring the blockade current s of the translocating analyte passed through the nanopore, comparing the experimental blockade current with a blockade current standard and determining the analyte, etc. Any of these steps can be used in the method of this invention and the person skilled in the art knows how to use any of these steps in the method of this invention. This invention is characterized in that the protein used is embedded with metal. Metal embedded protein nanopore amplifies the bolckage io current of the analyte because the channel of the nanopore is narrowed by metal adhesion.

In this invention, for the purpose of identifying an analyte, the protein nanopore may be positioned between a first conductive liquid medium and a second conductive liquid medium, wherein the nanopore comprises an opening that provides liquid communication. Optionally, the first conductive liquid medium and the second conductive liquid medium may be the same or different, either one or both may comprise one or more of a salt, a detergent, or a buffer. For example, in this invention, the first conductive liquid medium and the second conductive liquid medium may be the same and comprises 1.5 M KCl buffer consisting of 1.5 M KCl and 10 mM Tris-HCl at pH=7.0.

In this invention, the protein nanopore may be within a membrane such as a lipid bilayer. The membrane may be positioned between a first conductive liquid medium and a second conductive liquid medium.

The analyte is electrophoretically translocated through the nanopore by virtue of the electrical field that is applied to the nanopore. Process and apparatus for applying an electric field to a nanopore are known to the person skilled in the art. For example, a pair of electrodes may be used to applying an electric field to a nanopore. The electrical field is sufficient to translocate an analyte through the nanopore. As will be understood, the voltage range that can be used can depend on the type of nanopore system and the analyte being used. For example, in some embodiments, the applied electrical field is between about 20 mV and about 200 mV, for protein nanopores. In some embodiments, the applied electrical field is between about 60 mV and about 200 mV. In some embodiments, the applied electrical field is between about 100 mV and about 200 mV. In some embodiments, the applied electrical field is about 180 mV and about 200 mV.

As known by the person skilled in the art, when the analyte translocates through the channel of the nanopore, it interacts with the nanopore, causing a change in the current through the nanopore, which is usually a significant current reduction, known as blockade current. Different molecules will cause different blockade current, which could be used to characterize the composition information about the analyte passing through the nanopore. In general, a “blockade” is evidenced by a change in ion current that is clearly distinguishable from noise fluctuations and is usually associated with the presence of an analyte molecule within the nanopore. The strength of the blockade, or change in current, will depend on a characteristic of the analyte. The person skilled in the art can distinguish which kind of current change is blockade.

In this invention, more particularly, a “blockade” may refer to an interval where the ionic current drops to a level which is about 5-100% lower than the unblocked current level, remains there for a period of time, and returns spontaneously to the unblocked level. For example, the blockade current level may be about, at least about, or at most about 5%, 10%, 15%, 20%, 25%, 30%, 35%, 40%, 45%, 50%, 55%, 60%, 65%, 70%, 75%, 80%, 85%, 90%, 95%, or 100% lower than the unblocked current level.

Methods of measuring blockade current are well known in the art. Measurement of the blockade current through the nanopore may be performed by way of optical signal or electric current signal. For example, one or more measurement electrodes could be used to measure the current through the nanopore. These can be, for example, a patch-clamp amplifier or a data acquisition device. For example, Axopatch-IB patch-clamp amplifier (Axon 200B, Molecular Devices) could be used to measure the electric current flowing through the nanopore.

Those skilled in the art know how to determine the characteristics of an analyte based on the measured blockade current. For example, after the measured blockade current is obtained, said measured blockade current is compared with the blockade current standard and determining the analyte. For example, when the analyte is metal-containing analyte, the measured blockade current is compared with the blockade current standard of metal-containing analyte under the same testing conditions and determining whether the analyte is metal-containing analyte. For example, when the analyte is nucleic acid, the measured blockade current is compared with the blockade current standard of A, T, C and/or G or a combination thereof under the same testing conditions and determining the composition of the nucleic acid. For example, when the analyte is amino acid or peptide, the measured blockade current is compared with the blockade current standard of the amino acid or the peptide under the same testing conditions and determining the type of the amino acid or the composition of the peptide.

The method of the present invention can be qualitative or quantitative. Thus, the method of present invention can be used to determine the identity of the analyte, or the concentration of the analyte. In some embodiments, the method of the present invention can be used to determine the identity of the analyte such as metal-containing ion (e.g. [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn^{2°}, Hg²⁺, Ru(II) or Pb²⁺) or amino acid. For example, when the analyte is metal-contain analyte such as metal-containing ion (e.g. [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) or Pb²⁺), the measured blockade current is compared with the blockade current standard of metal-containing ion (e.g. [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺⁺, Ru(II) or Pb²⁺) under the same testing conditions and determining the identity of metal-containing ion (e.g. [AuCl₄]⁻, Zn²⁺, Co²⁺, Ag⁺, Cd²⁺, Ni²⁺, [AUCl₂]⁻, Cu²⁺, Cr³⁺, Fe²⁺, Fe³⁺, Pt(II), Pd(II), Mn²⁺, Hg²⁺, Ru(II) or Pb²⁺). For example, when the analyte is amino acid, the measured blockade current is compared with the blockade current standard of the amino acid under the same testing conditions and determining the identity of the amino acid. In some embodiments, the method of the present invention can be used to determine the concentration of the analyte such as metal-containing ion (e.g. [AuCl₄]⁻, Zn²⁺, Co²⁺, Ag⁺, Cd²⁺, Ni²⁺, [AuCl₂]⁻, Cu²⁺, Cr³⁺, Fe², Fe³⁺, Pt(II), Pd(II), Mn²⁺, Hg²⁺, Ru(II) or Pb²⁺) or amino acid. For example, when the analyte is metal-containing analyte such as metal-containing ion (e.g. [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) or Pb²⁺), the measured blockade current is compared with the blockade current of metal-containing ion (e.g. [AuCl₄]⁻, Zn²⁺, Co²⁺, Ag⁺, Cd²⁺, Ni²⁺, [AuCl₂]⁻, Cu²⁺, Cr³⁺, Fe²⁺, Fe³⁺, Pt(II), Pd(II), Mn²⁺, Hg²⁺, Ru(II) or Pb²⁺) having standard concentration(s) under the same testing conditions and determining the concentration of metal-containing ion (e.g. [AuCl₄]⁻, Zn²⁺, Co²⁺, Ag⁺, Cd²⁺, Ni²⁺, [AuCl₂]⁻, Cu²⁺, Fe²⁺, Fe³⁺, Pt(II), Pd(II), Mn²⁺, Hg²⁺, Ru(II) or Pb²⁺). For example, when the analyte is amino acid, the measured blockade current is compared with the blockade current of the amino acid having standard concentration(s) under the same testing conditions and determining the concentration of the amino acid.

According this invention, a method of identifying a metal-containing ion in a sample is provided, the method comprising:

providing a MspA positioned between a first conductive liquid medium and a second conductive liquid medium, wherein the MspA comprises an opening that provides liquid communication between the first conductive liquid medium and the second conductive liquid medium, wherein the first conductive liquid medium or the second conductive liquid medium comprises the sample;

applying an electric field across the MspA and translocating the metal-containing ion through the nanopore;

measuring the blockade current across the nanopore; and

identifying the metal-containing ion in the sample according to the measured blockade current.

According this invention, a system or device of identifying a metal-containing ion in a sample is provided, the system contains a MspA positioned between a first conductive liquid medium and a second conductive liquid medium, wherein the MspA comprises an opening that provides liquid communication between the first conductive liquid medium and the second conductive liquid medium, wherein the first conductive liquid medium or the second conductive liquid medium comprises the sample.

According this invention, a method of identifying an analyte in a sample is provided, the method comprising:

providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample, wherein the nanopore is embedded with one or more metal-containing ions;

applying an electric field across the nanopore and translocating the analyte through the nanopore;

measuring the blockade current across the nanopore; and

identifying the analyte in the sample according to the measured blockade current.

According this invention, a system or device of identifying an analyte in a sample is provided, the system or device contains a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample, wherein the nanopore is embedded with one or more metal-containing ions.

The interaction between the embedded metal (such as one or more [AuCl4]⁻¹ ions) and the amino acids on the inner surface of the nanopore can be achieved by applying an electric field and translocating the with metal-containing ion in the nanopore. Thus, in some embodiments, the metal-containing ions may be comprised in the first conductive liquid medium or the second conductive liquid medium together with the analyte to be detected, both the metal-containing ion and the analyte are translocating through the nanopore under the electric field, said metal-containing ion is bound to the inner surface of the nanopore and the analyte is identified.

Thus, one particular embodiment of the method of identifying an analyte in a sample io comprising:

providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample and metal-containing ions;

applying an electric field across the nanopore and translocating the metal-containing ions and the analyte through the nanopore;

measuring the blockade current across the nanopore; and

identifying the analyte in the sample according to the measured blockade current.

Thus, one particular embodiment of the system or device of identifying an analyte in a sample contains a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample and metal-containing ions.

In another embodiment, first, the metal-containing ions are allowed to bind to the inner surface of the nanopore, then, the sample is added into the first conductive liquid medium and the second conductive liquid medium and is translocating through the nanopore. Thus, another particular embodiment of the method of identifying an analyte in a sample comprising:

providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises metal-containing ions;

applying an electric field across the nanopore and translocating the metal-containing ion through the nanopore;

adding the sample into the conductive liquid medium which comprises the metal-containing ions initially and translocating the analyte through the nanopore;

measuring the blockade current across the nanopore; and

identifying the analyte in the sample according to the measured blockade current.

Thus, another particular embodiment of the system or device of identifying an analyte in a sample contains a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises metal-containing ions.

In some embodiments, said metal-containing ions which will be bound to the inner surface of the nanopore contain Au(III) such as Au³⁺. In some embodiments, said metal-containing ions are tetrachloroaurate(III) ions (that is [AulC₄]⁻). In some embodiments, chloroauric acid is added into the first or the second conductive liquid medium to form the metal-containing ions. In some embodiments, the concentration of [AuCl4]⁻¹ or chloroauric acid in the first conductive liquid medium or the second conductive liquid medium is equal to or greater than 200 nM, equal to or greater than 1 μM, equal to or greater than 15 μM, or equal to or greater than 10 μM.

In some embodiments, the final concentration of the metal-containing ions in the first conductive liquid medium or the second conductive liquid medium is at least 200nM, at least 1 μM, at least 4 μM, at least 5 μM, at least 8 μM, at least 10 μM, at least 15 μM, at least 50 μM.

The invention also relates to a kit for identifying an analyte, the kit containing: (1) metal-containing compound; and (2) a protein that can form a nanopore or a nucleic acid, expression vector or recombinant host cell that can express a protein that can form a nanopore.

Said metal-containing compound is capable of forming a metal-containing ion in a solution. Said metal-containing ion include metal ion and complex ion formed by metal and other ions. In some embodiments, said metal-containing compound is capable of forming Au(III), Zn²⁺, Co²⁺, Ag⁺, Cd²⁺, Ni²⁺, Aum, Cu²⁺, Cr³⁺, Fe²⁺, Fe³⁺, Pt(II), Pd(II), Mn²⁺, Hg²⁺, Ru(II) or Pb²⁺ in a solution. In some embodiments, said metal-containing compound is capable of forming [AuCl₄]⁻ for [AuCl₂]⁻ ion in a solution. In some embodiments, said metal-containing compound may be chloroauric acid or tetrachloroaurate(III).

Said protein that can form a nanopore is defined as the same with the above description of protein nanopore.

The embodiments described herein can be understood more readily by reference to the following detailed description, examples, and claims, and their previous and following description. It is to be understood that the embodiments described herein are not limited to the specific uses, methods, and/or products. It is also to be understood that the terminology used herein is for the purpose of describing particular aspects only and is not intended to be limiting.

Further, the following description is provided as an enabling teaching of the various embodiments in their best, currently known aspect. Those skilled in the relevant art will recognize that many changes can be made to the aspects described, while still obtaining the beneficial results of this disclosure. It will also be apparent that some of the desired benefits of the present invention can be obtained by selecting some of the features of the various embodiments without utilizing other features. Accordingly, those who work in the art will recognize that many modifications and adaptations to the various embodiments described herein are possible and can even be desirable in certain circumstances and are a part of the present disclosure. Thus, the following description is provided as illustrative of the principles of the embodiments described herein and not in limitation thereof.

Throughout this application, the term “about” is used to indicate that a value includes the standard deviation of error for the system or method being employed to determine the value. In any embodiment discussed in the context of a numerical value used in conjunction with the term “about,” it is specifically contemplated that the term “about” can be omitted.

It should be understood throughout the present specification that expression of a singular form includes the concept of their plurality unless otherwise mentioned. Accordingly, for example, it should be understood that a singular article (for example, “a”, “an”, “the” in English) comprises the concepts of plural form unless otherwise mentioned.

It should be also understood that the terms as used herein have definitions typically used in the art unless otherwise mentioned. Thus, unless otherwise defined, all scientific and technical terms have the same meanings as those generally used by those skilled in the art to which the present invention pertains. If there is contradiction, the present specification (including the definition) precedes.

All patents and publications, including all sequences disclosed within such patents and publications, referred to herein are expressly incorporated by reference.

### EXAMPLES

### Example 1

### Observing Single tetrachloroaurate(III) Binding Within a WT α-HL

The heptameric WT α-HL is a mushroom-shaped ion channel protein with a narrow cylindrical stem with an aperture of −1.4 nm in diameter at its narrowest point[9]. Due to the limited acquisition bandwidth (100 kHz) of a patch clamp amplifier (Axon 200B, Molecular

Devices), translocations of single inorganic ions through nanopores are not resolvable unless an interaction between the ion and the pore can be established. Based on the known sulfur-gold (S—Au) coordination interaction[24], methionine(113)[25-27] which is in the proximity of the 1^{st} restriction site of the pore[28] and is the only sulfur-containing amino acid within the inner surface of an α-HL monomer, is expected to form a reversible interaction with freely translocating tetrachloroaurate(III) ions crossing the membrane.

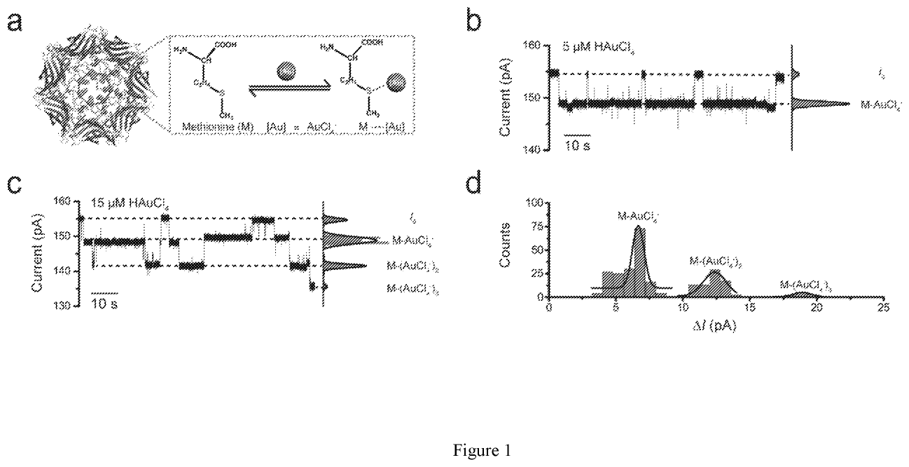

Experimentally, all electrophysiology measurements were performed with a patch clamp amplifier (Axon 200B, Molecular Devices) in an aqueous buffer consisting of 1.5 M KCl and 10 mM Tris-HCl at pH 7.0, unless otherwise stated (Methods). Chloroauric acid is added into the cis chamber (the side which is electrically grounded) to reach the desired final concentration. With a single WT α-HL inserted in the membrane, the anionic [AuCl4]⁻¹ is electrophoretically driven through the pore. At +100 mV, when chloroauric acid is added to the cis chamber reaching a 5 μM final concentration, binding of [AuCl4]⁻¹ generates characteristic resistive pulse signals (ΔI≈6 pA, τ_{off}≈10-20 seconds, Table 1) (FIG. 1, 2). Here ΔI stands for the resistive pulse amplitude and τ_{off} stands for the dwell time of the event.

TABLE 1
Statistics of ΔI₁ measured with α-HL WT.

| Independent | Current blockade | Dwell time |
|---|---|---|
| experiments | (ΔI₁) (pA) | (τ_{off}) (s) |
| 1 | 5.39 ± 0.67 | 13.30 ± 19.63 |
| 2 | 4.71 ± 0.71 | 26.43 ± 36.40 |
| 3 | 6.16 ± 1.03 | 7.57 ± 8.04 |

The measurements are taken with 1 μM HAuCl₄ at 100 mV and ΔI₁ stands for the amplitude difference between I₀ and M-(AuCl₄⁻)₁.

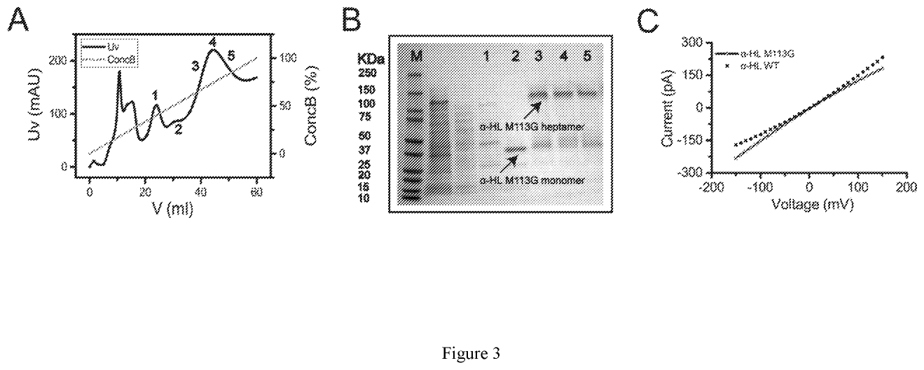

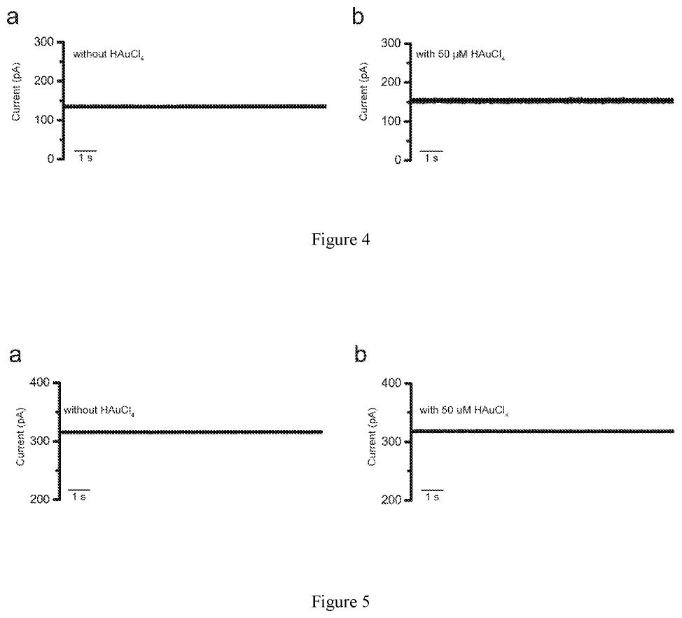

To verify this sensing mechanism, a Met→Gly mutation (M113G) was introduced by pore engineering (FIG. 3, Methods). No [AuCl₄]⁻ ion binding events as had been observed with WT α-HL were detected by α-HL M113G, even if the cumulative chloroauric acid concentration in cis is raised to 50 μM (FIG. 4). The absence of ion binding events in the M113G mutant is thus evidence for a coordination interaction between Au(III) and methionine (113) within the WT α-HL.

To the best of our knowledge, this is the first observation of single molecule coordination interaction between Au(III) and methionine in a confined pore restriction, although systematic investigations on the topic have previously been performed in ensembles[25-27]. Binding of tetrachloroaurate(III) in α-HL shows a wide dispersion in the statistics of the blockage amplitude (FIG. 1), which may result from non-specific binding of tetrachloroaurate(III) with other residues, such as K147 and K131, within the long and cylindrically shaped restriction of WT α-HL[13]. This long restriction complicates further engineering of α-HL for tetrachloroaurate(III) sensing. However, this newly discovered mechanism may be adapted to other channel proteins for enhanced sensing performances.

### Example 2

### Transplanting the tetrachloroaurate(III) Ion Binding Capability into MspA

Inspired by nanopore sequencing, where a nanopore with a single, geometrically sharp restriction, as in MspA[22] or CsgG[29] is advantageous because it has a higher spatial resolution[3], direct single ion sensing could also be performed with a geometrically sharp nanopore to acquire an enlarged signal amplitude and avoid non-specific binding with residues distant from the recognition site. The mutant M2 MspA[22] (D93N/D91N/D9ON/D118R/D134R/E139K), which was the first reported nanopore for DNA sequencing, is a funnel shaped, octameric ion channel protein which is ˜1.2 nm in diameter at its narrowest spot (Methods)[30]. The mutations in M2 MspA are designed to neutralize the original negative charges of WT MspA (PDB ID: luun[31]) for an enhanced capture rate for anions, such as DNA[22] or tetrachloroaurate(III). Based on a visual analysis of the corresponding protein structure, no methionine or cysteine exists within the inner surface of M2 MspA, making it a clean “template” to which methionine can be introduced by pore engineering.

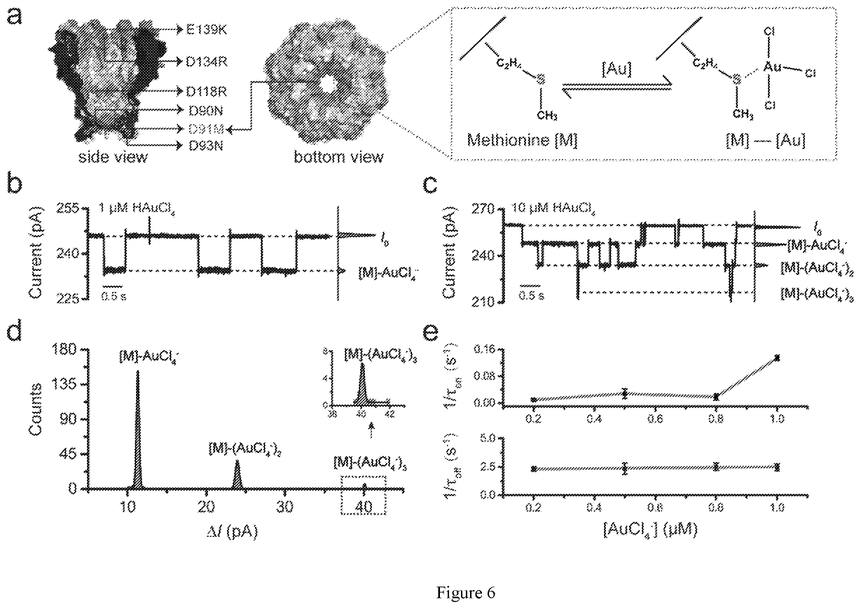

Experimentally, as in α-HL M113G (FIG. 4), no [AuCl4]⁻¹ binding events were detected by M2 MspA even when the cumulative chloroauric acid concentration in cis was raised to 50 μM (FIG. 5). By single site directed mutagenesis in the M2 MspA, a methionine was introduced at residue 91 (Methods), which at 1.2 nm in diameter[23] is the narrowest spot of MspA (FIG. 6a). This MspA mutant namely MspA-M,

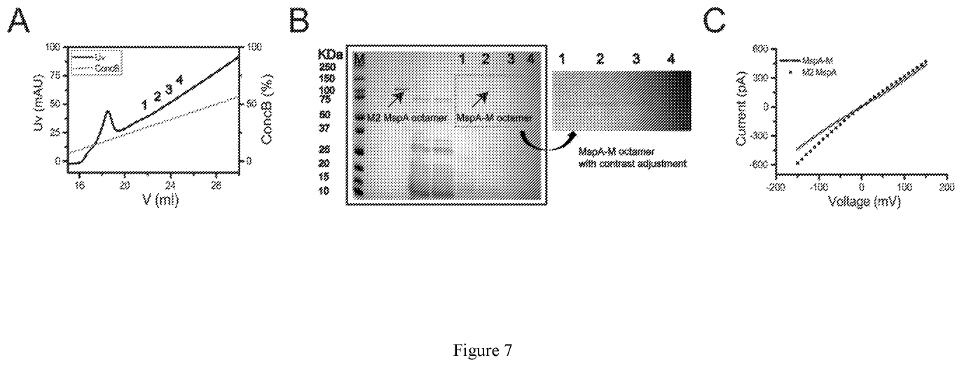

(D93N/D91M/D9ON/D118R/D134R/E139K), is prepared in the same way as its predecessor (M2 MspA) (Methods) and shows similar channel properties during its characterization (FIG. 7), indicating an octameric pore assembly which was unaltered by the mutation.

During continuous electrophysiology recording in 1.5 M KCl buffer with +100 mV applied potential (Methods), [AuCl₄]⁻¹ blockage events of MspA-M (FIG. 6b) appear when the final concentration of HAuCl₄ in cis reaches 200 nM. Significantly increased event counts are observed when the HAuCl₄ concentration in cis reaches 1 μM (FIG. 6b, Table 2).

TABLE 2
Statistics of ΔI₁ measured with MspA-M

| Independent | Current blockade | Dwell time |
|---|---|---|
| experiments | (ΔI₁) (pA) | (T_{off}) (s) |
| 1 | 11.26 ± 0.30 | 0.40 ± 0.36 |
| 2 | 11.23 ± 0.45 | 0.48 ± 0.41 |
| 3 | 12.49 ± 0.52 | 0.37 ± 0.49 |

The measurements are taken with 1 μM HAuCl₄ at 100 mV. ΔI₁ stands for the amplitude difference between I₀ and M-(AuCl₄⁻)₁.

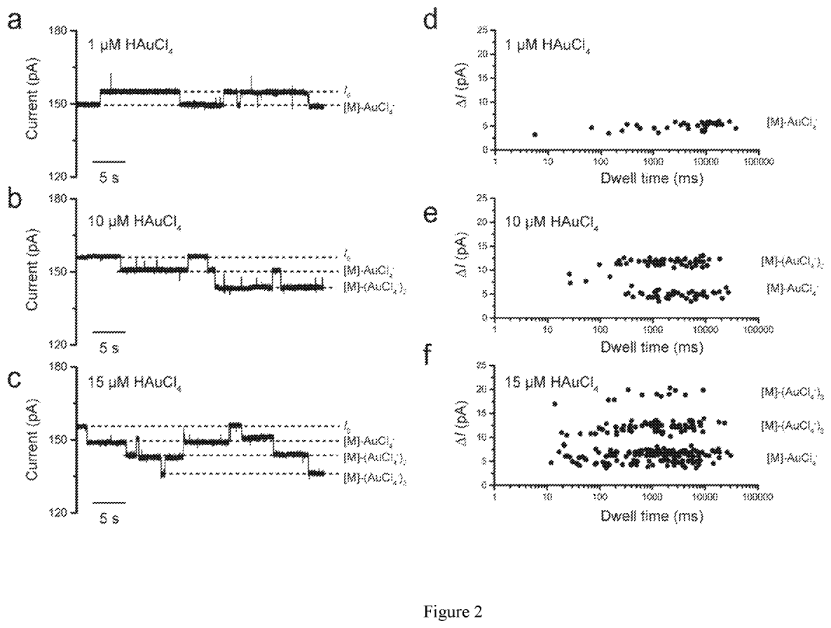

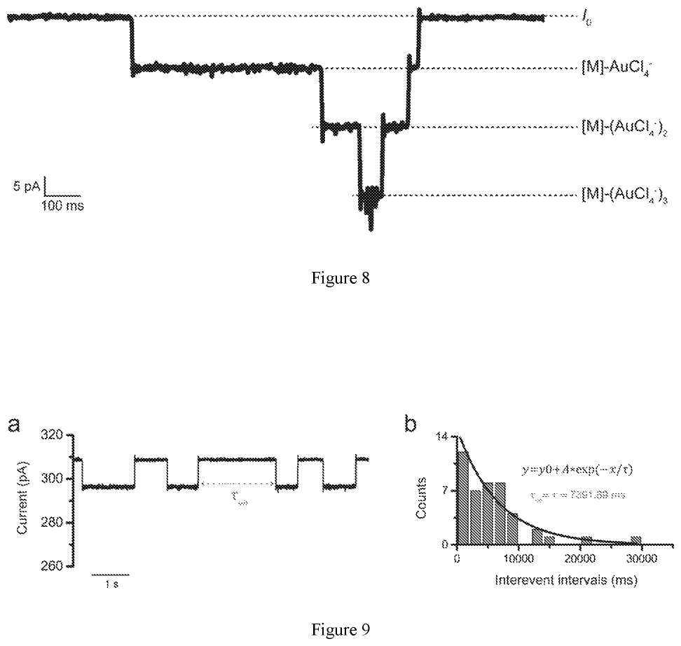

Considering the existence of eight identical methionine residues as a consequence of the octameric asymmetry of the pore, multi-level blockage events with approximately equal spacing gradually appear when the HAuCl₄ concentration is increased further, (FIG. 6c), indicating simultaneous blockages from more than one [AuCl₄]⁻¹ ion within the same pore. These blockage levels are named M-(AuCl₄⁻)ₙ, where n stands for the number of [AuCl₄]⁻¹ ions io in the pore at the same time (FIG. 6b, c). Similar to [AuCl4]⁻¹ sensing with WT α-HL (FIG. 2), direct transition from M—(AuCl₄⁻¹)ₙ to M-(AuCl₄⁻)_{n±2} is never observed (FIG. 8). This similarity between WT α-HL and MspA-M is evidence for a successful transplantation of the [AuCl4]⁻¹ sensing capability from WT α-HL to MspA-M.

The statistics of event blockage amplitudes, derived from a representative trace with 10 is minutes of continuous recording, show fully resolved peaks, in the form of narrow Gaussian peaks (FIG. 6d) corresponding to different M—(AuCl₄⁻¹)ₙ blockages. Non-specific events such as statistical counts not obeying the Gaussian distribution, are apparently never observed (FIG. 6d). Presumably, signal contributions from possible non-specific interactions distant from the restriction are weakened as negligible “off-focus” contributions, and this suggests the significant advantage of using a geometrically sharp ion channel for sensing single ions or small molecules, where pore engineering is simplified to a reduced amount of amino acids around the pore restriction in comparison to reported work with α-HL[13]. A high risk of reduced expression yield or failed pore assembly from highly mutated oligomeric channel proteins is sometimes encountered.

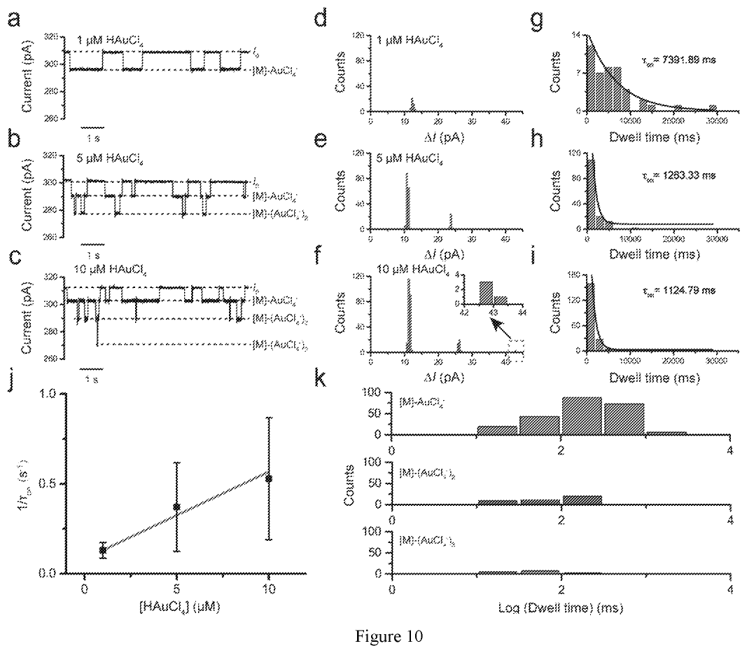

Though tetrachloroaurate(III) events are detectable with a 200 nM HAuCl₄ concentration in cis, a sharply increased detection frequency is observed (FIG. 6e) when the accumulated HAuCl₄ concentration in cis reaches 1 μM. However, the 1/τ_{off} value for M-(AuCl₄⁻)₁ events remains constant with all HAuCl₄ concentrations, indicating the same type of binding with the various analyte concentrations. When the HAuCl₄ concentration is further increased to 1-15 μM, M-(AuCl₄⁻)_{n>1} events become dominant (FIG. 6c), from which the statistics of τₒₙ, (time of inter-event duration, FIG. 9) and the corresponding on-rate can be deduced (FIG. 10, Table 3).

TABLE 3
The association rate constant for [AuCl₄]⁻ with MspA-M

| Concentration | 1/τₒₙ | Association rate |
|---|---|---|
| (μM) | (s⁻¹) | constant (M⁻¹ s⁻¹) |
| 1 | 0.13 ± 0.044 | Kₒₙ = (8.55 ± 4.23) × 10⁵ |
| 5 | 0.37 ± 0.25 | |
| 10 | 0.53 ± 0.34 | |

N = 3 to form the statistics.

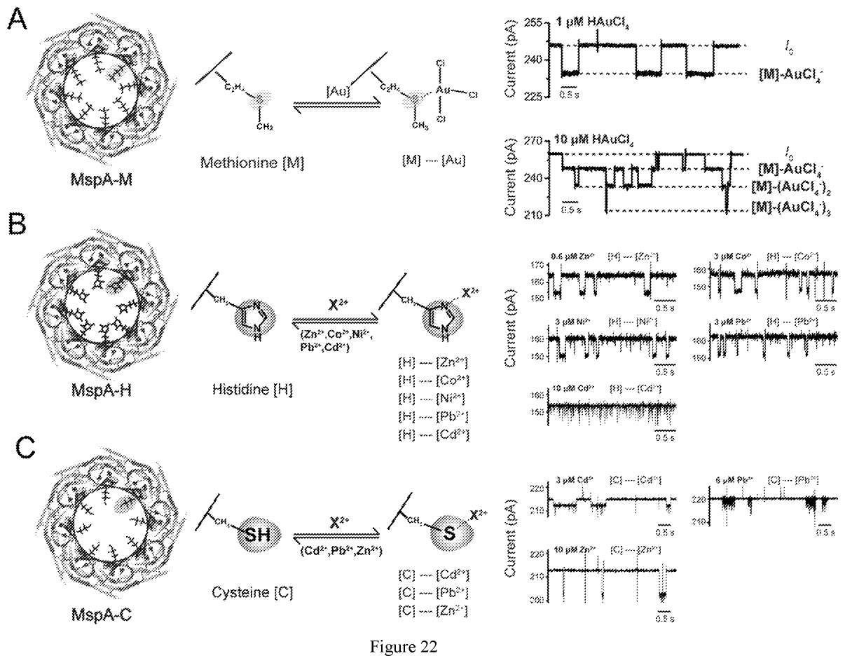

Embedding different metal ion types within an engineered MspA nanopore is shown in FIG. 22.

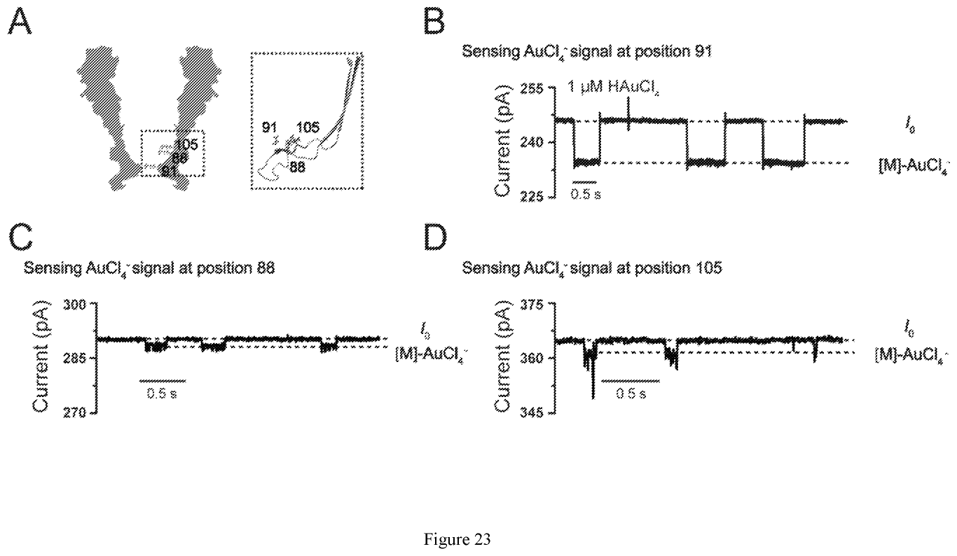

Engineered MspA nanopore at various locations for AuCl₄⁻ embedment is shown in FIG. 23.

### Example 3

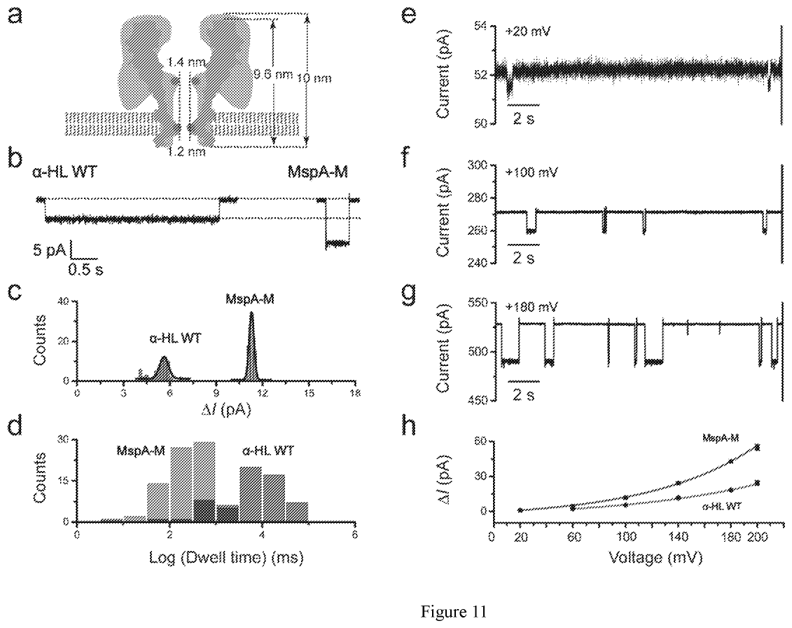

Single tetrachloroaurate(III) ion binding in different confined spaces Single tetrachloroaurate(III) binding has so far been demonstrated with two types of channel proteins possessing similar outer dimensions but different geometries (FIG. 11a). Technically, with appropriate protein engineering, any biological nanopore could be adapted to bind [AuCl₄]⁻¹ with this newly discovered mechanism. However, [AuCl₄]⁻¹ binding within different pores may generate varying single ion behaviors when differently restricted by for is example, geometry, charge or electric field in the vicinity of the restriction. These phenomena may suggest how further optimization of single ion sensors could be performed, or inspire the design of new gold-containing compounds as drug molecules which target ion channel proteins with high specificity.

Though the experiment is performed identically, [AuCl₄]⁻¹ blockage events in WT a-HL appear as shallow (ΔI=5.63±0.33 pA) resistive pulses but with longer duration and a wide dispersion (12.30±19.23 s) (FIG. 1). [AuCl₄]⁻¹ binding in MspA-M on the other hand, shows deeper blockage (ΔI=11.25±0.19 pA) but the event is shorter and has a much narrower distribution (0.40±0.36 s) (FIG. 11b). By analyzing the ΔI of M-(AuCl₄⁻)₁ events from WT α-HL or MspA-M, these differences are systematically observed in independent measurements by different researchers (Table 1, 2). Though a larger absolute value of ΔI is observed from MspA-M, the percentage blockage (ΔI/I₀) from the two pores are quite similar. We conclude that this is an effect of the pore geometry, in which the narrowest regions of each of the two pores, where tetrachloroaurate(III) binds, are similar in size (FIG. 11a) and determine the ΔI/I₀. The wider opening of MspA, which leads to a higher open pore conductance, results in the larger ΔI in the absolute amplitude. The larger ΔI amplitude along with a much narrowed dispersion (FIG. 11c) shows that MspA is superior to α-HL for single ion sensing. The conical geometry results simultaneously in a faster potential drop and a stronger electric field (E_{z}=−dV/dz). The enhanced electrophoretic force and electro-osmotic flow should contribute to the shorter duration time of tetrachloroaurate(III) binding in MspA-M (FIG. 11d).

The local charge distribution within the inner surface of the pore is critical for analyte attraction. It was found that MspA-M captures tetrachloroaurate(III) more efficiently than WT α-HL, where a 200 nM detection limit is observed from MspA-M, which is 5 times lower than that from α-HL. This may result from the positive charges introduced around the larger vestibule (D118R/D134R/E139K) of MspA-M, which was originally designed to attract ssDNA. [22] Similar phenomena are observed with other biological nanopores, when excessive positive charges in the pore lead to a more efficient DNA capture rate[32-34].

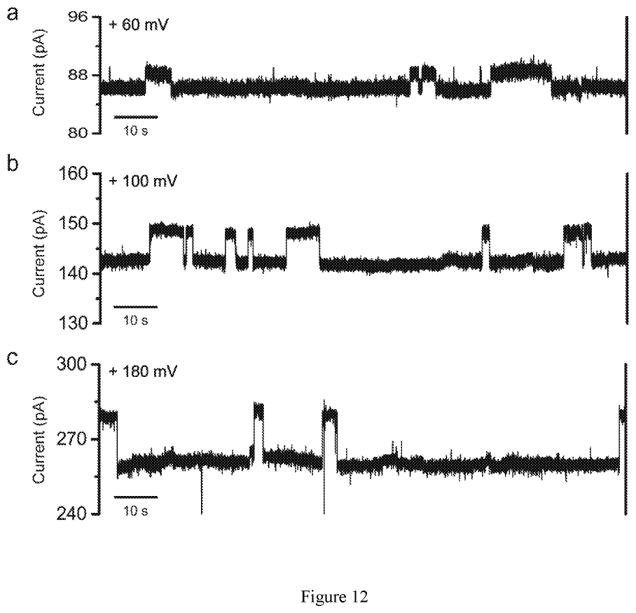

By measuring the voltage dependence for M-(AuCl₄⁻)₁ binding events, acquired with either WT α-HL (FIG. 12) or MspA-M (FIG. 11e-g), a maximum ΔI=˜54.88 pA is achieved with MspA-M at +200 mV, far beyondany pore blockage signals from a single ion that have previously been reported (FIG. 11h)[10, 12-14]. For [AuCl₄]⁻ in particular, binding at +200 mV. MsDA-M outweighs WT α-HL by ˜30.49 DA (FIG. 11h. Table 41.

TABLE 4
Mean current blockade at different voltages

| | Current blockade | Current blockade | |
|---|---|---|---|
| Voltage | (ΔI) for | (ΔI) for | Difference |
| (mV) | MspA-M (pA) | WT α-HL (pA) | of ΔI (pA) |
| 20 | 0.90 ± 0.08 | N.A. | N.A. |
| 60 | 4.18 ± 1.18 | 2.18 ± 0.33 | 2.00 |
| 100 | 11.66 ± 0.72 | 5.42 ± 0.60 | 6.24 |
| 140 | 24.31 ± 0.73 | 11.63 ± 0.85 | 12.68 |
| 180 | 42.85 ± 0.53 | 18.28 ± 0.49 | 24.57 |
| 200 | 54.88 ± 2.37 | 24.39 ± 1.77 | 30.49 |

On the contrary, [AuCl₄]⁻¹ binding in WT α-HL generates significant baseline fluctuations when recorded with more than +100 mV potential bias (FIG. 12), which limits its uses at high applied voltages. This phenomenon should result from non-specific binding of [AuCl₄]⁻¹ within the cylindrical stem of α-HL, when an increased number of anions are driven into the io pore constriction at high applied voltages. In low voltage measurements, though barely detectable (0.90±0.08 pA), [AuCl₄]⁻¹ binding within MspA-M is still visible at a +20 mV applied potential (FIG. 11e), a minimum of +60 mV is needed for WT α-HL to resolve the events. In summary, using [AuCl₄]⁻¹ as a model analyte MspA-M clearly outperforms WT a-HL in many aspects such as larger event amplitude, narrower event dispersion, higher sensing is specificity and lower detection limit.

Example 4

### ssDNA Translocation Through tetrachloroaurate(III) Embedded MspA

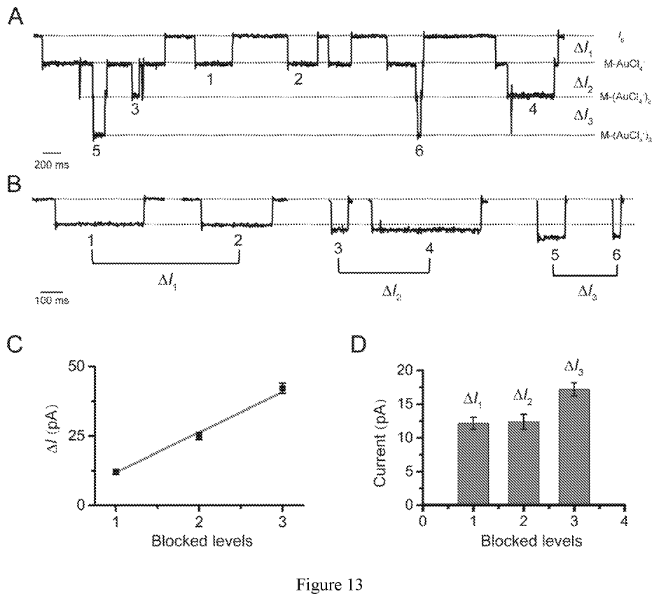

By quantitative analysis of the peak amplitude differences (ΔIₙ=I_{M}-(AuCl4̅)ₙ−I_{M}-(AuCl4̅)ₙ₋₁) in the event statistics (FIG. 6d), where M-(AuCl₄⁻)₀ stands for the open pore current (I₀), it can be seen that a rule, ΔI₃>ΔI₂>ΔI₁ is systematically observed (FIG. 13) for all independent experiments that have been completed (Table 5).

TABLE 5
Mean current blockade and peak amplitude differences

| Blockade | Absolute Current | peak amplitude |
|---|---|---|
| level | blockade (pA) | differences |
| M-AuCl₄⁻ | 12.20 ± 0.88 | ΔI₁ = 12.20 ± 0.86 |
| M-(AuCl₄⁻)₂ | 24.97 ± 1.34 | ΔI₂ = 12.43 ± 1.12 |
| M-(AuCl₄⁻)₃ | 42.16 ± 1.92 | ΔI₃ = 17.20 ± 0.98 |

Here ΔIₙ=I_{M-(AuCl}_{₄}_{^{−}}₎_{ₙ}−I_{M-(AuCl}_{₄}_{^{−}}₎_{_{n−1}}.

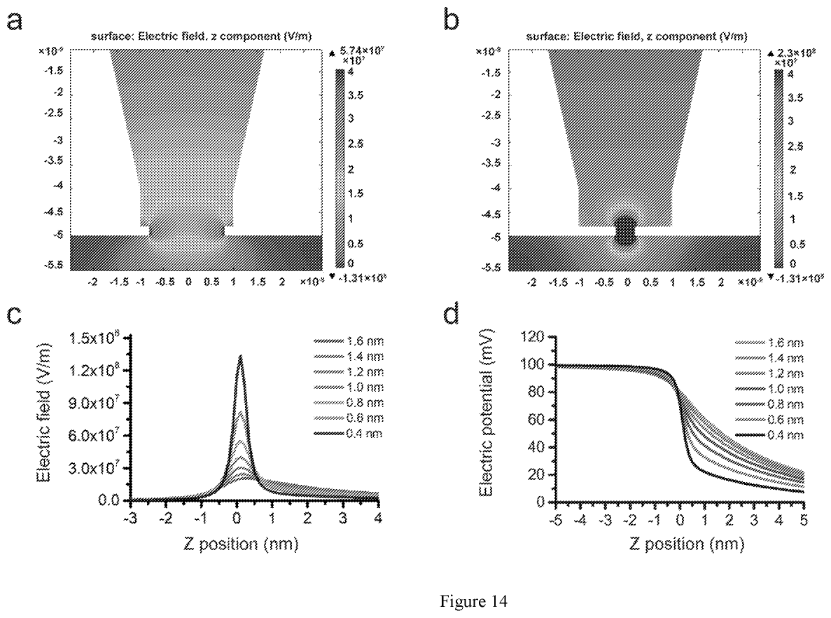

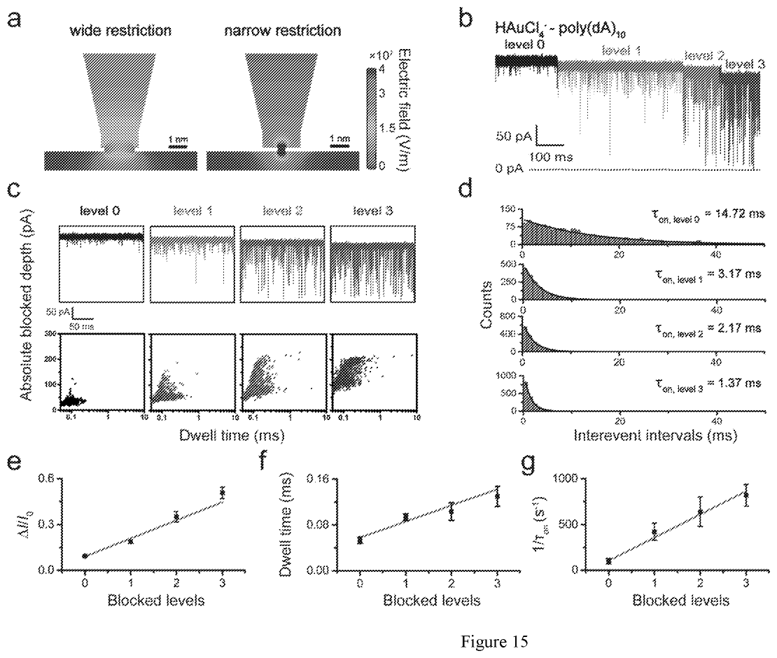

Presumably, sequential adhesion of [AuCl₄]⁻¹ ions gradually narrows the remaining pore cavity of MspA, making the pore restriction even sharper with the result that any further [AuCl₄]⁻¹ blockage signal is amplified with respect to previously accumulated [AuCl4]⁻¹ binding. This phenomenon could be explained semi-quantitatively using finite element method (FEM) modeling by Comsol (FIG. 14). Briefly, a conically shaped pore geometry is established with varying restriction sizes. It can then be demonstrated that a pore geometry with a much narrowed restriction results in an increased electric field strength around the restriction site, making it more sensitive to analyte binding (FIG. 15a). This means that a biological pore embedded with more than one [AuCl₄]⁻, could resolve further [AuCl₄]⁻¹ binding even more effectively.

Originally developed for DNA sequencing purposes', fine tuning of the restriction geometry of MspA has not been reported to date. For an [AuCl₄]⁻¹ embedded MspA-M, the blockage remains <10%, leaving 90% of the pore cavity open, and possibly still permits ssDNA to translocate. Amplified ssDNA sensing signals are anticipated from the MspA with a narrowed restriction due to the Au(III) embedment. A DNA homopolymer poly(dA)₁₀ (Table 6) was selected as a model analyte to translocate through MspA with a dynamically narrowed Au embedment. Such a short ssDNA homopolymer was selected to avoid complications resulted from DNA secondary structures.

TABLE 6
Nucleic acid abbreviations and sequences

| Abbreviation | Sequences (5′-3′) |
|---|---|
| Poly(dA)₁₀ | AAAAAAAAAA |
| 78-nt | AAGCAGACAGGCGGGAAGGTTTTTTTTCTAGAGGGG |
| | AATTGTTATCCGCTCACAATTCCCCTATAGTGAGTC |
| | GTATTA |

Experimentally, chloroauric acid and poly (dA)₁₀ were added to the cis reaching a final concentration of 10 μM and 8 μM respectively. The electrophysiology recording was carried out at +100 mV in 1.5 M KCl buffer (Methods). As demonstrated in FIG. 15b, sequential [AuCl₄]⁻¹ binding with MspA-M slightly reduces its remaining open pore current, termed leveln, where n is the number of [AuCl₄]⁻¹ ions simultaneously in the pore (FIG. 6). On the other hand, ssDNA translocation signals show up with larger pore blockage amplitudes and detection frequencies for levelₙ with larger n values (FIG. 15b). Considering that poly(dA)₁₀ translocation signals are barely detectable at level₀, the [AuCl₄]⁻ ion embedded MspA-M nanopore shows the advantages of an increased sensitivity and amplified signals due to ion embedding in the finely tuned pore cavity.

A scatter plot of ssDNA translocation events extracted from different leveln is presented in FIG. 15c, in which significantly increased event counts of high-amplitude blockages are observed with leveln of larger n values. The increased event counts are verified by statistics of τₒₙ, (inter-event duration for ssDNA translocation) in which a 5-10 fold increased capture rate is observed (FIG. 15d). Though widely distributed in the scatter plot (FIG. 15c) even with an ion-embedded pore, the mean percentage blockage ΔI/I₀ amplitude and the mean dwell time of ssDNA translocation events clearly show a linear increase with increasing n values (FIG. 15e-g, Table 7), indicating a significant modulation for ssDNA translocation characteristics with atomic tuning of the restriction.

TABLE 7
Poly(dA)₁₀ statistics

| Blockade level | 1/τₒₙ (s⁻¹) | ΔI/I₀ | Mean dwell time (ms) |
|---|---|---|---|
| 0 | 99.30 ± 29.36 | 0.093 ± 0.006 | 0.053 ± 0.006 |
| 1 | 420.87 ± 93.25 | 0.187 ± 0.011 | 0.093 ± 0.006 |
| 2 | 638.77 ± 161.24 | 0.350 ± 0.035 | 0.103 ± 0.015 |
| 3 | 819.17 ± 117.57 | 0.507 ± 0.038 | 0.130 ± 0.017 |

The association rate constant and the mean dwell time and ΔI/I₀ for poly(dA)₁₀ with MspA-M in the presence of 10 μM HAuCl₄. N=3 to form the statistics.

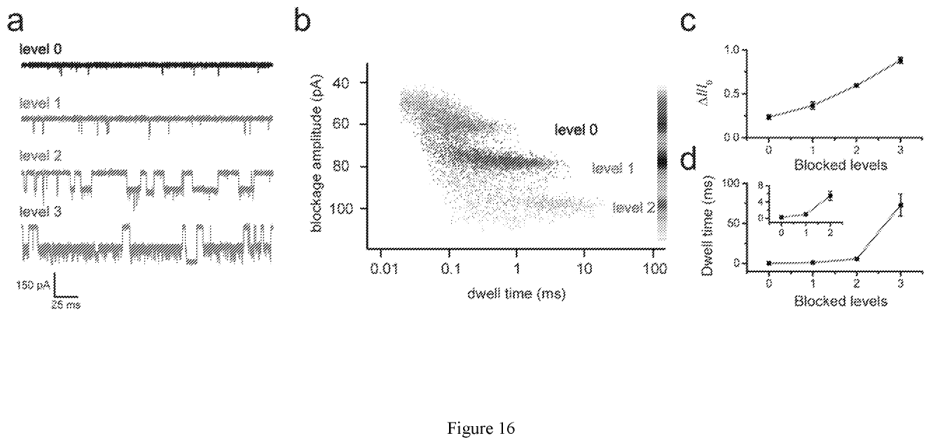

As reported previously[23], a minimum length of ssDNA (>50 nucleotides) is required for io ssDNA capturing into the MspA nanopore, which has limited its direct sensing applications to short oligomeric nucleic acids, such as miRNA, siRNA or short DNA probes. Pore restriction modulation by dynamic Au(III) embedment demonstrated efficient sensing of short nucleic acid oligomers as short as 10 nucleotides in length. A similar phenomenon is also observed with a 78 nucleotide ssDNA composed of a random sequence (FIG. 16, Table 8), which indicates that the MspA with a narrowed pore restriction is in general more sensitive with ssDNA sensing.

TABLE 8
78-nt event statistics

| Blockade level | ΔI/I₀ | Mean dwell time (ms) |
|---|---|---|
| 0 | 0.23 ± 0.027 | 0.23 ± 0.025 |
| 1 | 0.37 ± 0.042 | 0.97 ± 0.17 |
| 2 | 0.59 ± 0.019 | 5.52 ± 1.14 |
| 3 | 0.88 ± 0.035 | 72.81 ± 13.98 |

The mean dwell time and ΔI/I₀ for 78-nt ssDNA with MspA-M in the presence of 10 μM HAuCl₄.

As reported, the M2 MspA, though possessing a conical geometry, fails to truly resolve a single nucleotide without entanglement with signals from adjacent bases[3], but relies on complicated bioinformatics programs1 for sequence decoding. The accuracy of the decoding is still unsatisfactory compared to next generation sequencing platforms. Nanopore sequencing may be carried out in a MspA nanopore with atomic tuning, where a tighter pore restriction may produce a higher spatial resolution, reduce thermal fluctuations from molecular vibrations or produce more signal characteristics for single nucleotide recognition. However, further engineering of pores with permanent atomic embedment becomes important.

Example 5

### Highly Specific L-methionine Sensing by Au(III) Embedded MspA

Besides modulating the size of the pore restriction, Au(III) embedment also endows the MspA nanopore with new recognition functionalities, such as highly specific sensing of L-methionine by using the embedded Au(III) atom as an atomic adaptor. Although significant attention has been paid to how a protein can be sequenced using nanopores[35], an immediate challenge is to gain the pore restriction with a sensing specificity that fully discriminates 20 amino acids directly from pore blockage events.

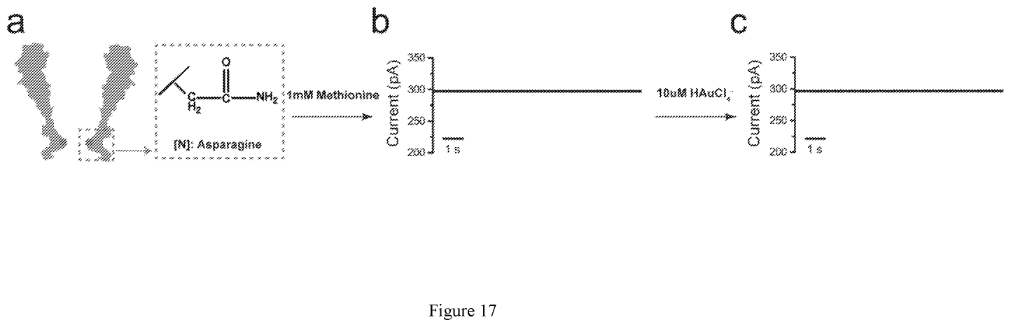

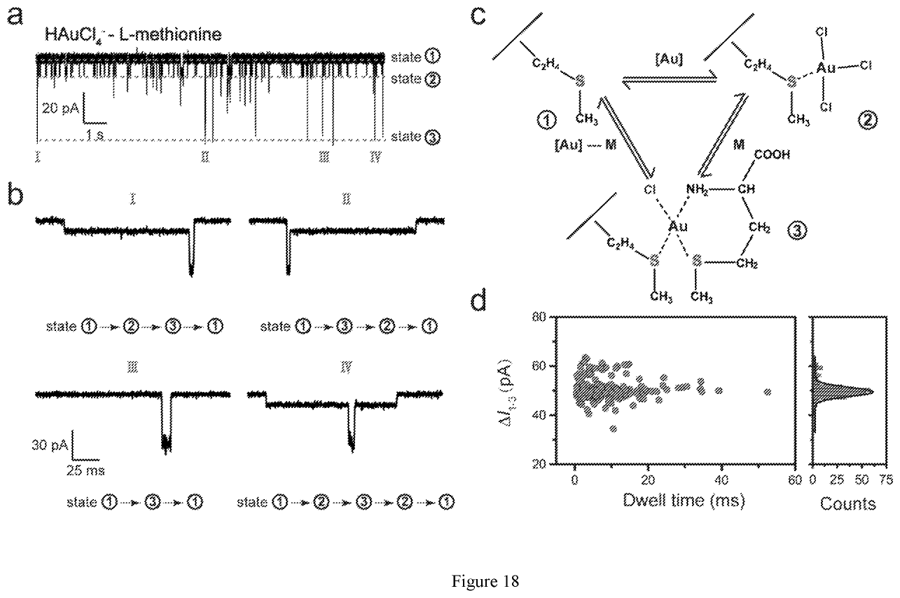

Experimentally, the M2 MspA does not report any signal for L-methionine or tetrachloroaurate(III) (FIG. 17) as no interaction can be established between either analyte and the pore. The mutant MspA-M, which has a single N91M mutation in reference to M2 MspA, interacts strongly with tetrachloroaurate(III) (FIG. 6) but reports no direct sensing io signal for L-methionine. However, during continuous recording in 1.5 M KCl buffer with +100 mV applied potential (Methods), MspA-M reports deep blockage events (˜48 pA) when L-methionine and chloroauric acid were added to the cis with 80 μM and 4 μM final concentration, respectively (FIG. 18a). These deep blockages are never observed when chloroauric acid is added as the sole analyte (FIG. 6). Detailed inspections indicate that these is deep blockage events could be categorized into four types, which can be understood as various combinations of three event states (FIG. 18b).

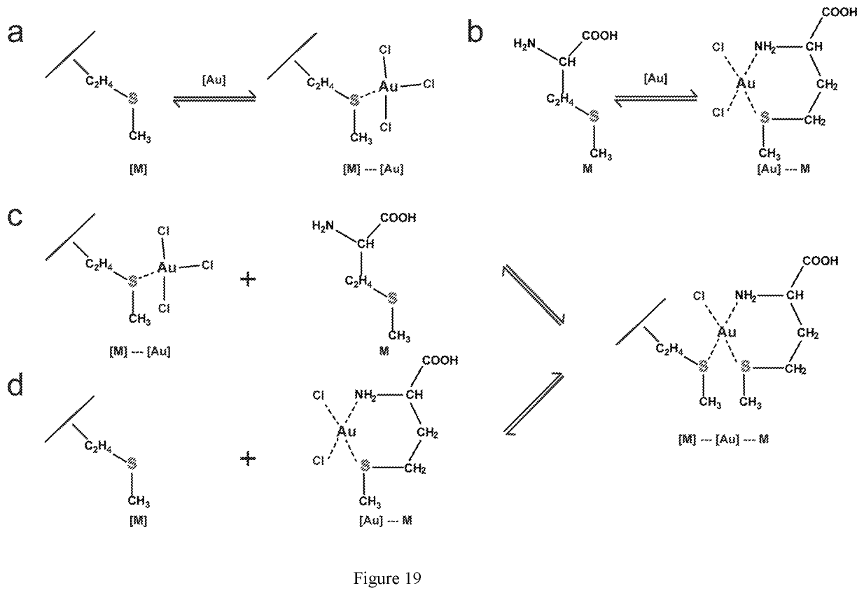

A molecular model which interprets all four types of L-methionine induced blockage events is demonstrated in FIG. 18c. From this model, state 3 represents the bound state of L-methionine with site 91 using Au(III) as a bridge atom (FIG. 19). A scatter plot was generated by taking the duration time of state 3 and the amplitude change between state 1 and 3 (ΔI₁₋₃) to demonstrate the event statistics (FIG. 18d). To avoid complications from multiple tetrachloroaurate(III) binding, only events with one tetrachloroaurate(III) binding are included in the statistics. From the corresponding amplitude histogram, ΔI₁₋₃ for all four types of events overlap and are distributed around 48 pA. Three independent measurements show that ΔI₁₋₃ measures 48.67±0.91 pA in amplitude with a dwell time of 10.05±1.73 ms (Table 9).

TABLE 9
Statistics for L-Methionine sensing

| Independent | Current blockade | Dwell time |
|---|---|---|
| experiments | (ΔI₁₋₃) (pA) | (τ_{off}) (s) |
| 1 | 47.69 ± 0.17 | 8.07 ± 3.18 |
| 2 | 49.49 ± 0.09 | 10.87 ± 1.87 |
| 3 | 48.82 ± 0.90 | 11.22 ± 2.35 |

Statistics of ΔI₁₋₃.for L-methionine with MspA-M in the presence of 4 μM HAuCl₄. at 100 mV. ΔI₁₋₃ stands for the amplitude difference between I₀ and [M]-(AuCl₄⁻)-M.

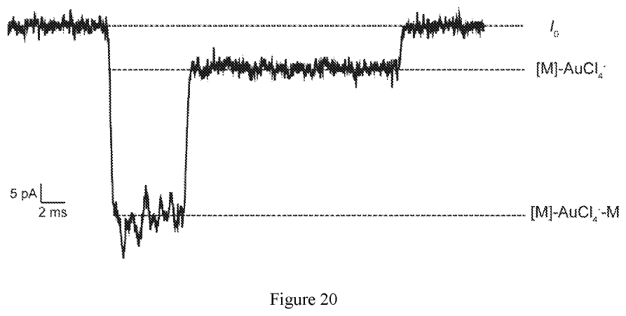

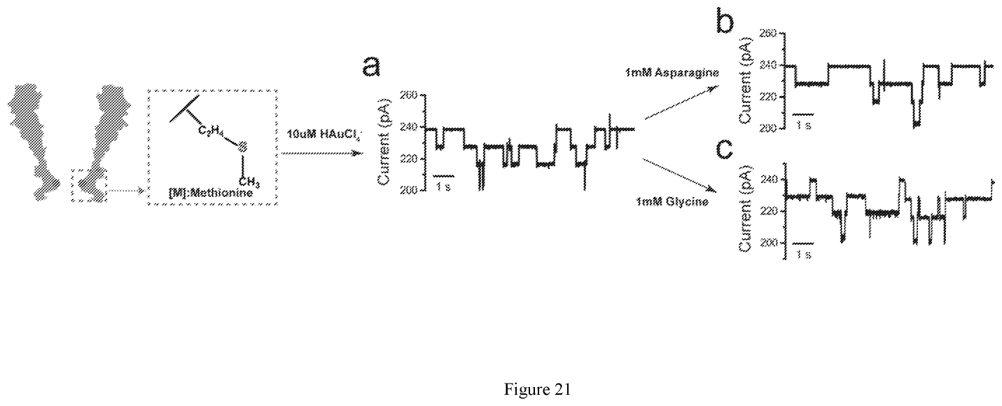

If single tetrachloroaurate(III) binding results in an ˜11 pA drop in the current under these measurement conditions (Table 2), the absolute blockage amplitude from single L-methionine is approximately 37 pA, about 10 times larger in amplitude than was observed in the previously reported amino acid sensing using engineered α-HL.[36, 37] This enlarged pore blockage amplitude from L-methionine results in part from the conical geometry of MspA[36]. However, the Au(III) embedment, which narrows the pore restriction, should also further amplify the pore blockage amplitude due to an increased electric field around the sensing spot where the analyte binds (FIG. 15a). Detailed inspections for blockage events at state 3 show consistent and characteristic level fluctuations (FIG. 20), which may originate from analyte vibrations or non-specific bond formation. Similar measurements with L-asparagine and L-glycine (FIG. 21) report no events at all like state 3, which indicates that the embedded Au(III) is highly specific to the sulfur atom on the amino acid side chain.

This demonstration suggests a new strategy for highly specific amino acid sensing or protein sequencing using nanopores with designed atomic adapters. However, dynamic binding and dissociation of tetrachloroaurate(III) embedment continues to complicate the data analysis. Further engineering with this approach may be done by permanently embedding metal ions using irreversible coordination interactions.[36] Full discrimination of other amino acids can be achieved with designed atomic adaptors targeting different side groups of the analyte amino acids. The locations of these adaptors within a conically shaped biological nanopore could also be widely dispersed to tune the signal amplitude and so optimize signal discrimination.

However, with inevitable existence of isomers, the octameric symmetry of MspA, purifications could be challenging if 2-6 Au(III) atoms are to be introduced into an MspA pore with particular positioning requirements. An alternative solution may simplify the situation by taking the monomeric channel protein OmpG[38-40] as a template for metal ion embedding. Other ion-amino acid combinations within a variety of biological nanopores such as Cytolysin A[41], Phi29 motor protein[42] or aerolysin[2] may also be adapted for different applications.

### Example 6

### Direct Sensing of L-cysteine by Au(III) Embedded MspA

When bound to a methionine, the Au(III) atom remains in the proximity of the restriction of MspA for ˜0.5 s, forming a transient Au(III) embedment as an adaptor for sensing. Besides the demonstrated Au(III)-thioether interaction, a stronger interaction between Au(III)-thiol is expected, as previously reported[53], which indicates that an Au(III) embedded MspA may sense a variety of thiol-containing molecules. The most abundant biothiols include L-cysteine (Cys), L-homocysteine (Hcy) and L-glutathione (GSH), which are directly involved in crucial physiological processes[54, 55, 56] such as protein synthesis[57], free radical scavenging[54] and normal immune system maintenance[58]. Though presented in the blood plasma with a high abundance, in the ˜μM range[59], the structure similarity of these biothiols presents a great challenge for a direct simultaneous discrimination. With distinct physiological roles, discriminative sensing of these biothiols could have great significance in biomedical diagnostics.

Conventionally, sensing of biothiols was performed with high performance liquid chromatography-mass spectroscopy[60] or designed fluorescence probes[61], but suffers from a time consuming and laborious sample preparation process or the challenge of probe design.

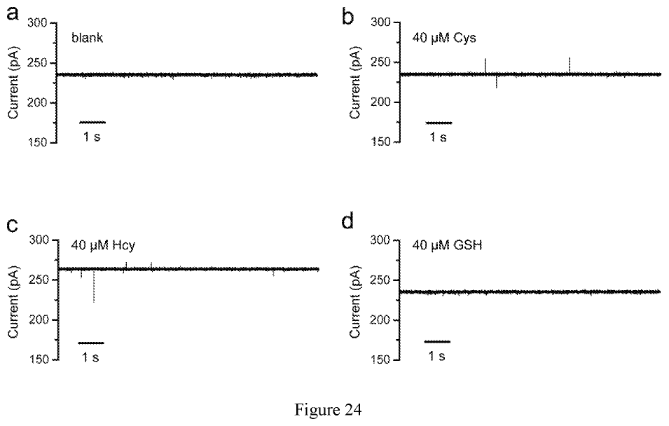

Nanopore sensing, which is inexpensive, fast and has advantages in the resolution of minor structural differences in small molecules, may provide an alternative solution for direct sensing of biothiols. However, a biological nanopore, such as an octameric MspA-M, doesn't directly report signals for all biothiols in general (FIG. 24) without the establishment of an interaction between the analyte and the pore.

On the other hand, the demonstrated Au(III) embedment enables MspA to interact with biothiols via the Au(III)-thiol coordination chemistry. The Au(III)-thiol coordination, which forms a much stronger bond than the established Au(III)-thioether coordination, competes with the existing Au(III)-thioether bond and consequently speeds up the dissociation of the Au(III) from the pore. Though the described chemical process happens rapidly, it can be monitored by a nanopore sensor, which forms the basis for sensing.

As a proof of concept, nanopore-based biothiol sensing was carried out with MspA-M as described in Methods. Specifically, HAuCl₄ was added to the cis while the biothiols were s added in the trans compartment. The two analytes were added to different sides of a nanopore to minimize the spontaneous redox reactions between Au(III) and biothiols before entering the pore restriction (FIG. 25a). With this configuration, the anionic [AuCl₄]⁻ was first electrophoretically driven into the pore where it binds to the methionine at the pore restriction. Subsequently, the bound Au(III), which acts as an atomic bridge, captures freely translocating io biothiol molecules and is stimulated to dissociate from the thioether group on the pore. Subsequently, a new sensing cycle is initiated whenever the next Au(III) binds (FIG. 25b).

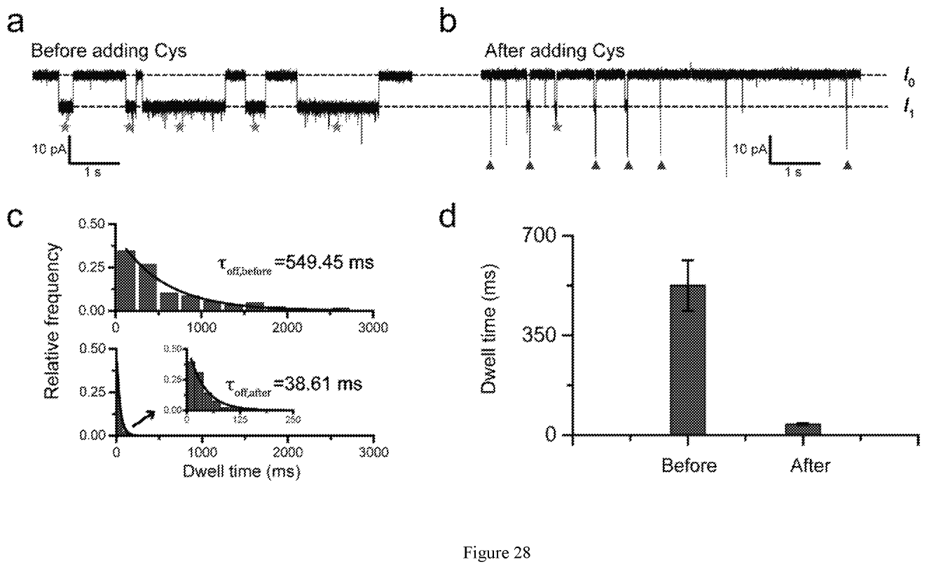

L-cysteine (Cys), which is an essential amino acid involved in protein synthesis[57], is the most well-known biothiol (FIG. 25c). As a test of feasibility, nanopore based biothiol sensing was performed (Methods) by adding chloroauric acid to the cis and Cys to the trans with 4 μM and 40 μM in concentration respectively. From electrophysiological recordings, MspA-M reported a new type, 2-step shaped blockage event (FIG. 25d), which could be clearly distinguished from binding events when HAuCl₄ was the sole analyte added (FIG. 11). A representative event of such type is composed of three states namely 0, 1 and 1_{SH}, which corresponds to the open pore state, the [AuCl₄]⁻ bound state and the Cys bound state, as described in the molecular model from FIG. 25b. State 1_{SH} can be recognized from its characteristic jitter signals, which have never been observed from [AuCl₄]⁻¹ binding events (FIG. 25e). The characteristic jitter signal might represent a redox reaction between Cys and the bound Au(III). Nonetheless, the interaction between Au(III) and a thiol group could be indirectly monitored from the significant reduction in the dwell time of state 1 when Cys was added in trans (FIG. 28). The newly formed Au(III)-thiol interaction has stimulated the dissociation of Au(III) from the nanopore.

With their characteristic event shape, Cys sensing events can be immediately distinguished from other non-specific binding types. To ensure the readability, only Cys sensing events were counted in the statistics which were based on a simple algorithm that an event has to contain all three states as demonstrated in FIG. 25e. Other non-specific event types, including binding and dissociation of one [AuCl₄]⁻¹ without any captured biothiol, sequential binding of two [AuCl₄]⁻¹ ions and intrinsic noises from MspA-M (FIG. 26) were ignored. Nevertheless, the Cys sensing event amounts to 96% of all detectable events from continuously recorded results (FIG. 27). A scatter plot of the relative blockage amplitude ΔI/I₀ versus the dwell time from the events of [AuCl₄]⁻ and Cys sensing is shown in FIG. 25f. Though with a slightly wider dispersion than that of [AuCl₄]⁻¹ (FIG. 25f), the Cys event forms a clear monodispersed distribution. This is clearly demonstrated in the histogram of ΔI/I₀ (FIG. 25g, Table 10, 11) and the dwell time τ_{off} (FIG. 25h), from which ΔI/I₀ of Cys can be seen to be 0.104±0.008 (Table 11, N=364) and the mean dwell time of state 1 _{SH} was derived as 3.18 ms (FIG. 25g-h).

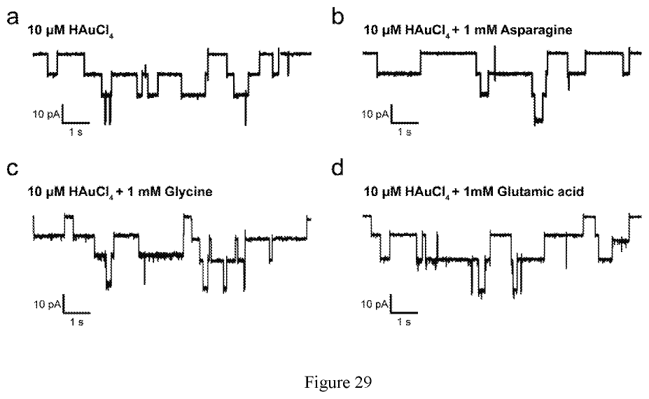

Similar measurements were also performed with L-asparagine, L-glycine and L-glutamic acid (FIG. 29) and no events such as that reported in FIG. 25e were observed. This indicates that this sensing configuration is highly specific to the thiol side chain of an amino acid.

TABLE 10
Statistics for L-Cysteine sensing.

| | Current | Dwell | Current | Dwell time | |
|---|---|---|---|---|---|
| Independent | blockade | time | blockade | (τ_{off}, _{Cys}) | ΔI₁₋₁, _{Cys} |
| experiments | (ΔI₀₋₁) (pA) | (τ_{off}) (ms) | (ΔI₀₋₁, _{Cys}) (pA) | (ms) | (pA) |
| 1 | 11.3 ± 0.5 | 35.51 | 25 ± 2 | 3.48 | 13.7 |
| 2 | 11.9 ± 0.7 | 38.61 | 25 ± 2 | 3.18 | 13.1 |
| 3 | 10.8 ± 0.3 | 41.23 | 25 ± 2 | 3.04 | 14.2 |

The statistical data were from 10 min continuous recordings with MspA-M when 4 μM HAuCl₄ were placed in cis and 40 μM Cys were placed in trans. A +100 mV voltage was applied. ΔI₀₋₁ stands for the amplitude difference between I₀ and I₁. ΔI_{0-1,Cys} stands for the amplitude difference between I₀ and I_{1,Cys}. τ_{off} stands for the dwell time of I₁, τ_{off,Cys} stands for the dwell time of I_{1,Cys}. Three independent measurements were performed for each condition to form the statistics.

TABLE 11
Statistics of ΔI₀₋₁, _{Cys}/I₀ for L-Cysteine sensing.

| Independent | Open current (I₀) | | |
|---|---|---|---|
| experiments | (pA) | ΔI₀₋₁/I₀ | ΔI_{0-1, cys}/I₀ |
| 1 | 240 | 0.047 ± 0.002 | 0.104 ± 0.008 |
| 2 | 240 | 0.049 ± 0.003 | 0.105 ± 0.008 |
| 3 | 238 | 0.045 ± 0.002 | 0.104 ± 0.007 |

The statistical data were from 10 min continuous recordings with MspA-M when 4 μM HAuCl₄ were placed in cis and 40 μM Cys were placed in trans. A +100 mV voltage was applied. ΔI₀₋₁ stands for the amplitude difference between I₀ and ΔI_{0-1,Cys} stands for the amplitude difference between I₀ and I_{a,Cys}. Three independent measurements were performed for each condition to form the statistics.

### Example 7

### Direct Sensing of L-Homocysteine by Au(III) Embedded MspA

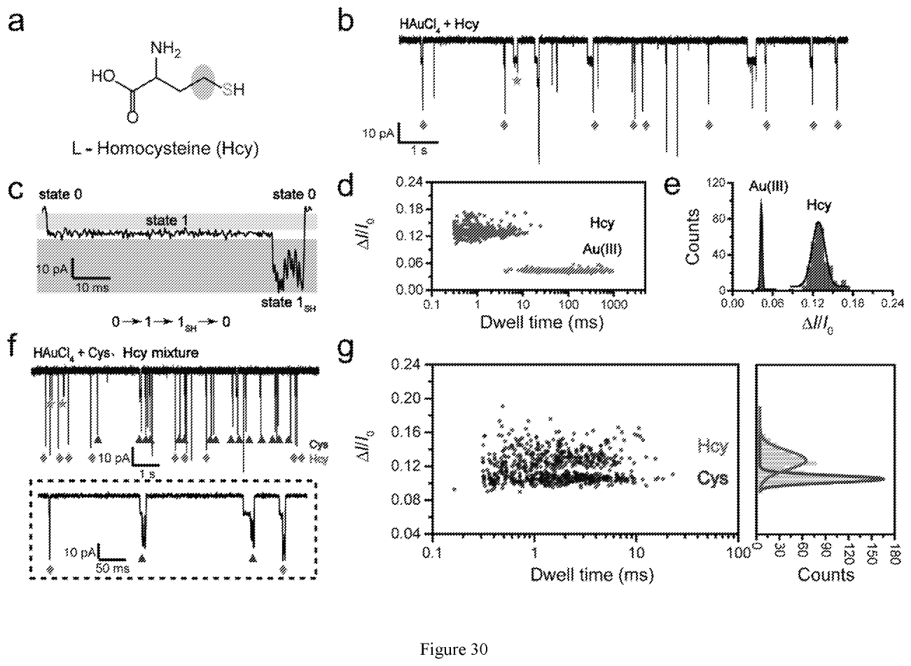

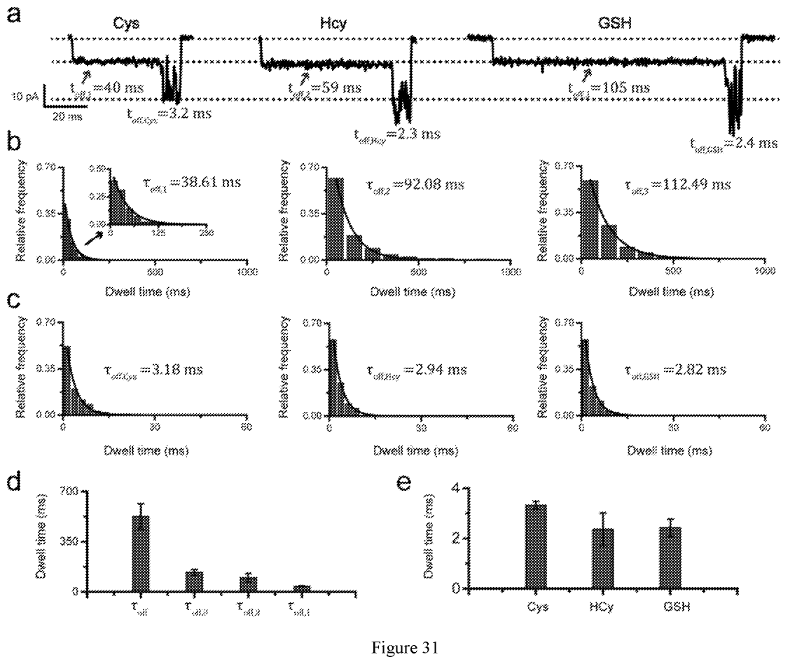

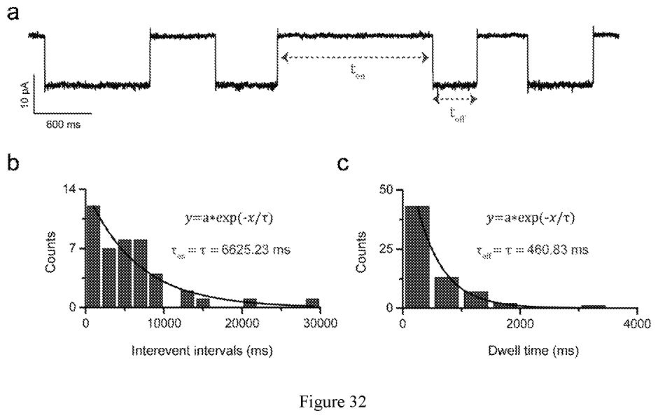

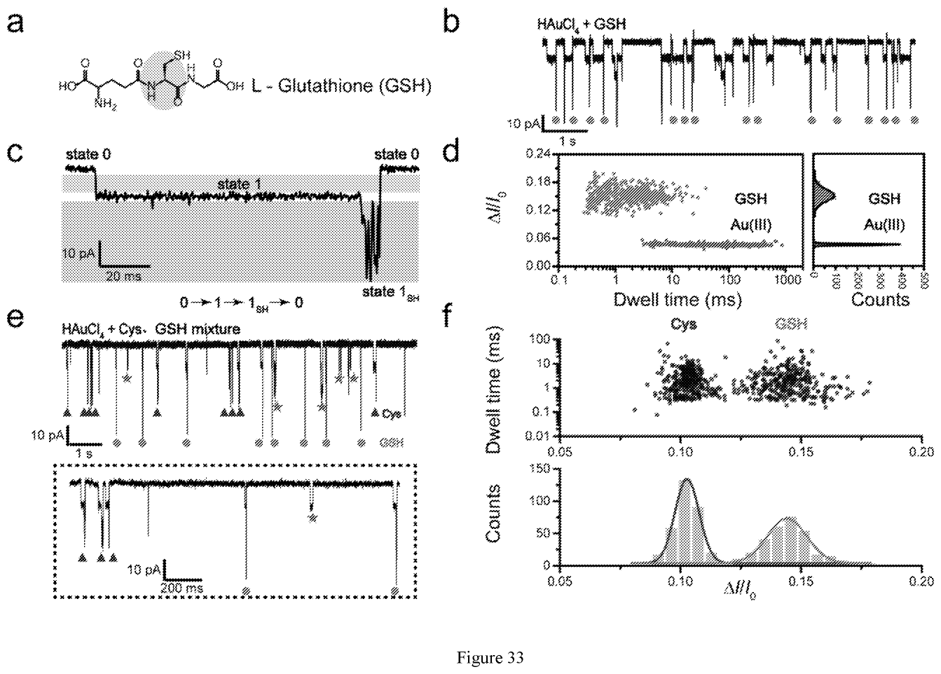

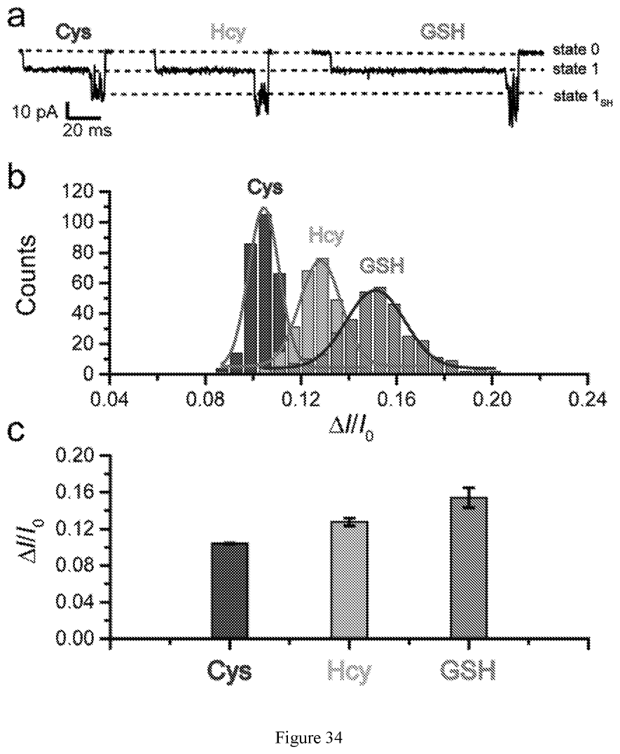

L-Homocysteine (Hcy), which is a homologue of Cys, is an important intermediate in the metabolism of methionine and cysteine[62]. An elevated Hcy level in the blood serum indicates a high risk of cardiovascular diseases and is a critical parameter in diagnosis[63]. However, Hcy differs from Cys with just one additional methylene group (FIG. 30a), and discrimination between Hcy from Cys is a challenge.

Hcy sensing was performed as described in FIG. 25a (Methods) when 4 chloroauric acid was placed in cis and 40 μM Hcy in trans. Systematically deeper blockage events compared with that of Cys (˜33 pA, marked by green squares) were observed from continuously recorded traces (FIG. 30b). A representative Hcy event is also composed of three states (FIG. 30c), similar to the behavior of Cys (FIG. 25e). These differ however in the I_{SH} state. A scatter plot of ΔI/I₀ vs the dwell time from events of [AuCl₄]⁻¹ and Hcy sensing was presented to show the event dispersion (FIG. 30d). From the corresponding histogram of ΔI/I₀, the Hcy blockage events measure 0.13±0.01 (N=330) (FIG. 30e, Table 12, 13) which confirms that a deeper relative blockage amplitude compared with that of Cys was observed. On the other hand, the mean dwell time of state 1_{SH} of Hcy measures 2.94 ms (Table 12), similar to that of Cys (FIG. 31).

To demonstrate simultaneous discrimination between Cys and Hcy from direct single molecule readouts, a nanopore measurement was performed with a mixure of 20 μM Cys and 20 μM Hcy in trans. The concentration of HAuCl₄ in cis was kept at 4 μM. The addition of two types of biothiols immediately reports two distinguishable event types as demonstrated from a continuously recorded trace (FIG. 30f). The scatter plot of ΔI/I₀ vs the dwell time with the io corresponding amplitude histogram (FIG. 30g) clearly shows two distinct event types. From the corresponding Gaussian fitting results, the two peaks in the histogram of ΔI/I₀ are 0.105±0.005 for Cys and 0.128±0.013 for Hcy respectively, which are in agreement with the separately measured values. The above demonstration suggests that a direct discrimination of Cys and Hcy, which differ by only one methylene group, is possible from direct nanopore readouts.

TABLE 12
Statistics for L-Homocysteine sensing.

| | Current | Dwell time | Current | Dwell time | |
|---|---|---|---|---|---|
| Independent | blockade | (τ_{off}) | blockade | (τ_{off, Hcy}) | ΔI_{1-1, Hcy} |
| experiments | (ΔI₀₋₁) (pA) | (ms) | (ΔI_{0-1, Hcy}) (pA) | (ms) | (pA) |
| 1 | 11.2 ± 0.3 | 92.08 | 33 ± 3 | 2.94 | 21.8 |
| 2 | 11.3 ± 0.5 | 130.72 | 34 ± 2 | 1.65 | 22.7 |
| 3 | 11.4 ± 0.5 | 71.94 | 35 ± 2 | 2.50 | 23.6 |

The statistical data were from 10 min continuous recordings with MspA-M when 4 μM HAuCl₄ were placed in cis and 40 μM Hcy were placed in trans. A+100 mV voltage was applied. ΔI₀₋₁ stands for the amplitude difference between /₀ and h. 0/₀₋₁,_{Hcy} stands for the amplitude difference between /₀ and l_{uicy}. . T_{off} stands for the dwell time of h, T_{off,cys} stands for the dwell time of I_{1,Hcy}. Three independent measurements were performed for each condition to form the statistics.

TABLE 13
Statistics of ΔI_{0-1, Hcy}/I₀ L-Homocysteine sensing.

| Independent | Open current (I₀) | | |
|---|---|---|---|
| experiments | (pA) | ΔI₀₋₁/I₀ | ΔI_{0-1, Hcy}/I₀ |
| 1 | 265 | 0.042 ± 0.001 | 0.123 ± 0.01 |
| 2 | 265 | 0.043 ± 0.002 | 0.129 ± 0.01 |
| 3 | 266 | 0.043 ± 0.003 | 0.131 ± 0.01 |

The statistical data were from 10 min continuous recordings with MspA-M when 4 μM HAuCl₄ were placed in cis and 40 μM Hcy were placed in trans. A +100 mV voltage was applied. ΔI₀₋₁ stands for the amplitude difference between I₀ and I₁. ΔI_{0-1,Hcy} stands for the amplitude difference between I₀ and I_{1,Hcy}. Three independent measurements were performed for each condition to form the statistics.

TABLE 14
Statistics for L-Glutathione sensing.

| | | Dwell | Current | | |
|---|---|---|---|---|---|
| | Current | time | blockade | Dwell time | |
| Independent | blockade | (τ_{off}) | (ΔI₀₋₁, _{GSH}) | (τ_{off}, _{Cys}) | ΔI₁₋₁, _{GSH} |
| experiments | (ΔI₀₋₁) (pA) | (ms) | (pA) | (ms) | (pA) |
| 1 | 10.8 ± 0.3 | 152.00 | 40 ± 6 | 2.19 | 29.2 |
| 2 | 10.9 ± 0.4 | 112.49 | 36 ± 3 | 2.82 | 25.1 |
| 3 | 10.7 ± 0.4 | 142.45 | 34 ± 4 | 2.27 | 23.3 |

The statistical data were from 10 min continuous recordings with MspA-M when 4 μM HAuCl₄ were placed in cis and 40 μM GSH were placed in trans. A +100 mV voltage was applied. ΔI₀₋₁ stands for the amplitude difference between I₀ and I₁. ΔI₁, ΔI_{0-1GSH} stands for the amplitude difference between I₀ and I_{1,GsH}. τ_{off} stands for the dwell time of I₁, τ_{off,Cys} stands for the dwell time of I_{1,GsH}. Three independent measurements were performed for each condition to form the statistics.

### Example 8

Direct sensing of L-Glutathione by Au(III) Embedded MspA

L-Glutathione (GSH), which is a tripeptide (Glu-Cys-Gly) (FIG. 33a), is critical in the maintenance of immune system. It is also the most abundant tripeptide thiol found in human serum[58]. As demonstrated with Cys and Hcy, the internal thiol in a GSH makes it compatible with the described biothiol sensing strategy.

With 4 μM chloroauric acid in cis and 40 μM GSH in trans, characteristic biothiol sensing events measuring ˜36 pA in amplitude were observed (FIG. 33b). From a representative event, the characteristic jitter signal of GSH (state 1_{SH}) reports a significantly larger blockage amplitude (FIG. 5c) than that of Cys (FIG. 25e). According to the negative control performed with glutamic acid and glycine separately (FIG. 29), it was confirmed that the internal cysteine of GSH is critical to the generation of the event. The scatter plot of ΔI/I₀ vs the dwell time with the corresponding amplitude histogram for GSH and [AuCl4]⁻¹ are shown in FIG. 5d. The ΔI/₀ of GSH measures 0.15±0.02 (Table 14, 15) with a mean dwell time of 2.82 ms in state 1_{SH} (FIG. 31, Table 14), indicating that it can be clearly distinguished from Cys via direct single molecule readouts.

Simultaneous discrimination of Cys and GSH was performed by adding a mixture of Cys and GSH in trans with 10 μM and 30 μM final concentrations respectively, while HAuChin cis remained at 4 μM. Compared with Cys or Hcy, GSH has a larger molecular weight and is negatively charged in a pH neutral buffer. With a +100 mV applied voltage, it was found that Cys is much more likely than GSH to be captured by the Au(III) embedded nanopore so that the GSH concentration in the mixture was increased to balance the rate of appearance of both events. From the electrophysiology trace, two types of events were clearly identified according to the difference in their amplitudes (FIG. 33e). The scatter plot of dwell time vs ΔI/I₀ with the corresponding amplitude histogram for GSH and Cys are shown in FIG. 33f, from which two populations of events were clearly visible. This unambiguously confirmed that Cys and GSH can be clearly discriminated from direct nanopore readouts.

### Example 9

### Comparison of Biothiol Sensing by Au(III) Embedded MspA

The sensing events of three types of biothiols are summarized in FIG. 34a. Though the [AuCl₄]⁻¹ states (state 1) appear identical, these events differ in the amplitude of their state 1sH. The addition of biothiols in the trans compartment also significantly reduces the binding time of a [AuCl₄]⁻¹ from a single molecule observation. Without addition of biothiols, the dwell time of a bound [AuCl₄]⁻¹ measures 530±90 ms. After the addition of Cys, Hcy or GSH in trans io with a 40 μM concentration, the dwell time of state 1 was immediately reduced, which indicates that the thiol group from a biothiol molecule strongly competes with the existed Au(III)-thioether interaction. The time reduction appears to be different and this may result from the differences in steric hindrance when Cys, Hcy or GSH interact with the Au(III). From the single molecule results, among the three tested biothiols, Cys reacts most strongly with is Au(III) (FIG. 31).

The histogram of ΔI/I₀ for Cys, Hcy and GSH events with corresponding Gaussian fittings are shown in FIG. 34b and an order of I_{1,GSH}>I -I_{Hcy}I_{1,Cys} can be clearly observed. This was expected because GSH is significantly larger than the other two amino acids and Hcy has an excess methylene when compared with Cys.

From the histogram in FIG. 34b, it is clear that Cys, Hcy and GSH can be clearly distinguished from their distinct ΔI/I₀ values. However, due mainly to the jitter signal of state I_{SH}H, the ΔI/I₀ value appears with a wide distribution and an inevitable signal overlap. Further optimization may be achieved by a permanent gold adaptor, which may extend the duration time of the state 1_{SH} for an eventual convergence of the event amplitude. However, statistical data of mean ΔI/I₀ histogram indicates that the distinction between Cys, Hcy and GSH can be achieved from independent measurements (FIG. 34c) (n=3 pores for each condition, Tables 11, 13 and 15). These results demonstrate that the Au(III) embedded MspA could serve as a highly sensitive probe for the differentiation of GSH, Hcy and Cys.

Distinct from a recent report of cysteine and homocysteine discrimination using nanopores[64], in which a time consuming sample preparation is needed and GSH was in principle not detectable, the described method in this invention suggests a strategy that is simple and straightforward, which could sense a wide variety of proteins containing biothiols or thiols using nanopores with designed atomic adapters. However, dynamic binding and dissociation of Au(III) embedment continues to complicate the data analysis, which makes it difficult to quantify biothiols reliably. Further engineering with this approach may be done by permanently embedding metal ions using irreversible coordination adaptors[65]. Full discrimination of other amino acids may be achieved with designed atomic adaptors targeting different side groups of the amino acid analytes. The locations of these adaptors within a conically shaped biological nanopore may also be slightly dispersed so that the signal amplitude from different analyte may be tuned to assist full discrimination.

TABLE 15
Statistics of ΔI_{0-1, GSH}/I₀ L-Glutathione sensing.

| Independent | Open current (I₀) | | |
|---|---|---|---|
| experiments | (pA) | ΔI₀₋₁/I₀ | ΔI_{0-1, GSH}/I₀ |
| 1 | 238 | 0.045 ± 0.001 | 0.167 ± 0.023 |
| 2 | 235 | 0.046 ± 0.002 | 0.151 ± 0.015 |
| 3 | 235 | 0.049 ± 0.003 | 0.145 ± 0.015 |

The statistical data were from 10 min continuous recordings with MspA-M when 4 μM HAuCl₄ were placed in cis and 40 μM GSH were placed in trans. A +100 mV voltage was applied. A/₀₋₁ stands for the amplitude difference between I₀ and I₁. ΔI_{0-1,GSH} stands for the amplitude difference between I₀ and I_{1,GSH}. Three independent measurements were performed for each condition to form the statistics.

### CONCLUSION

We have demonstrated the first polyatomic ion sensing using WT αHL. The observed io single molecule coordination interaction between Au(III) and methionine may inspire the design of new gold-containing compounds as drugs which target ion channel proteins. This molecular mechanism could also be transplanted into other biological nanopores such as MspA. As a consequence of geometric optimization, binding of single tetrachloroaurate(III) ions results in enlarged, consistent and sharply distributed blockage signals. The observed tetrachloroaurate(III) binding event in MspA at +200 mV also includes the largest single ion blockage signal (˜54.88 pA) to have ever been demonstrated. The sharp restriction of MspA along with the simplicity of mutagenesis suggests its role as a new engineering template to sense a wide variety of single ions and other small molecules, complementary to its well-known uses in nanopore sequencing.

When embedded with Au(III), the translocation characteristics of ssDNA could be gradually modulated when the restriction is atomically narrowed by further [AuCl₄]⁻ embedment as a consequence of geometric modulation. We also demonstrate highly specific recognition of L-methionine by MspA-M when embedded with a single tetrachloroaurate(III) ion, which suggests a new recognition functionalization strategy in nanopore based protein sequencing.

With its unique physical and chemical properties, gold has been used extensively in a wide range of scientific and industrial applications such as production of nanoparticles[43], tunneling electrodes[44], surface enhanced raman spectroscopy probes[45] and surface plasmonic resonance substrates[46]. However, these technologies lack single molecule control precision comparable to that offered by a biological nanopore, in which the geometry[47], orientation[48], polarity[41] and chemical modifications[10] of both the pore and the analyte can be manipulated. By taking the embedded Au(III) as an atomic bridge, MspA is enabled with biothiol-sensing capacities which directly discriminate between L-cysteine, L-homocysteine and L-glutathione from single molecule readouts. Though demonstrated as a proof of principle, this sensing mechanism is simple, label free, fast and economic and may be engineered into a portable sensor chip. With this first report of insertion of Au into an engineered biological nanopore with atomic precision and flexibility, this technology may benefit a wide range of scientific research projects in need of single molecule precision and the properties from gold[49, 50] or even other metal elements if properly designed.

### Methods

### Materials

Hexadecane, pentane, ethylenediaminetetraacetic acid (EDTA), Triton X-100, Genapol X-80 and hydrogen tetrachloroaurate (III) hydrate (99.99%) and L-Glutathione reduced were obtained from Sigma-Aldrich. 1,2-diphytanoyl-sn-glycero-3-phosphocholine (DPhPC) was from Avanti Polar Lipids. Dioxane-free isopropyl-P-D-thiogalactopyranoside (IPTG), to kanamycin sulfate, imidazole and tris(hydroxymethyl)aminomethane (Tris) were from Solarbio. 4-(2-hydroxyethyl)-1-piperazineethanesulfonic acid (HEPES) was from Shanghai Yuanye Biotechnology (China). E. coli strain BL21 (DE3) were from Biomed. LB broth and LB agar were from Hopebio (China). Hydrochloric acid (HC1) was from Sinopharm (China). L-methionine, L-asparagine, L-glycine, L-cysteine, and L-glutamic acid were from BBI Life Sciences (China). L-Homocysteine was from J & K Chemical Technology.

The potassium chloride buffer (1.5 M KCl, 10 mM Tris-HCl, pH 7.0) was prepared with Milli-Q water and membrane (0.2 μm, Whatman) filtered prior to use. HPLC-purified ssDNA (Genescript, New Jersey, Table 6) was dissolved in Milli-Q water without further purification. Hydrogen tetrachloroaurate (III) hydrate was dissolved in Milli-Q water as a stock solution (30 mM) for subsequent experiments. L-methionine, L-asparagine and L-glycine were dissolved in Milli-Q water as stock solutions (20 mM) for subsequent experiments. L-cysteine, L-asparagine, L-glycine, L-glutamic aicd L-homocysteine and L-glutathione reduced were dissolved in the potassium chloride buffer at 5 mM final concentration for subsequent experiments.

### α-HL Preparation

The gene coding for α-HL WT and α-HL M113G were custom synthesized and constructed in a pet 30a(+) plasmid (Genescript, New Jersey) for prokaryotic protein expression. Heptameric α-HL were expressed with E. coli BL21 (DE3) and purified with nickel affinity chromatography as previously published[51]. After heat shock transformation with plasmid gene coding for either α-HL WT or α-HL M113G, the cells were grown in LB medium at 37° C. till OD₆₀₀=0.7. Isopropyl P-D-thiogalactoside (IPTG) was then added to a final concentration of 1 mM for induction. After shaking overnight at 18° C., the cells were harvested by centrifugation (4000 rpm, 20 min, 4° C.). The pellet was collected and re-suspended in lysis buffer 1 (0.5 M NaCl, 20 mM HEPES, 1% Triton X-100, pH=8.0), sonicated for 15 min and then centrifuged (14,000 rpm, 4° C., 40 min) to remove intact cells. After syringe filtration, the supernatant was loaded onto a nickel affinity column (HisTrapTM HP, GE Healthcare). After washing the column with buffer Al (0.5 M NaCl, 20 mM HEPES, 5 mM imidazole pH 8.0), the α-HL heptamers were then eluted with a linear gradient of imidazole to buffer B1 (0.5 M NaCl, 20 mM HEPES, 500 mM imidazole, pH 8.0). Fractions of interests were further characterized and confirmed with SDS-polyacrylamide gel electrophoresis (FIG. 3). Only fractions of the α-HL heptamer were collected for subsequent electrophysiology.

### MspA Preparation

The gene codings for M2 MspA (D93N/D91N/D9ON/D118R/D134R/E139K) and MspA-M (D93N/D91M/D9ON/D118R/D134R/E139K) were custom synthesized and constructed in a pet 30a(+) plasmid (Genescript, New Jersey) for prokaryotic protein expression as previously published[51]. After heat shock transformation with plasmid gene coding for either M2 MspA or MspA-M, the cells were grown in LB medium to an OD600=0.7, induced with by 1 mM isopropyl P-D-thiogalactoside (IPTG) and shaken overnight at 16 ° C. The cells were harvested by centrifugation (4000 rpm, 20 min, 4° C.) and the pellet was re-suspended in lysis buffer 2 (100 mM Na₂HPO₄/NaH₂PO₄, 0.1 mM EDTA, 150 mM NaCl, 0.5% (w/v) Genapol X-80, pH 6.5), and heated to 60° C. for 10 min. The suspension was cooled on ice for 10 min and io centrifuged at 4° C. for 40 min at 13,000 rpm. After syringe filtration, the supernatant was applied to a nickel affinity column (HisTrapTM HP, GE Healthcare). After washing the column with buffer A2 (0.5 M NaCl, 20 mM HEPES, 5 mM imidazole, 0.5% (w/v) Genapol X-80, pH=8.0), bound proteins were eluted with a linear gradient of imidazole to buffer B2 (500 mM imidazole, 0.5 M NaCl, 20 mM HEPES, 0.5% (w/v) Genapol X- is 80, pH=8.0). The fractions for MspA octamer were collected and characterized by 12% SDS-PAGE (FIG. 7) and used for directly for electrophysiology measurements.

Electrophysiology Recording and data analysis

All electrophysiology results were acquired by an Axopatch 200B patch clamp amplifier and digitized by a Digidata 1550 Al digitizer (Molecular Devices, UK). A custom made measurement chamber is separated by a Teflon film (30 pm thick) with an orifice (6=100 pm). Before use, the orifice was pretreated with 0.5% (v/v) hexadecane in pentane and then air-dried to evaporate the pentane. 1,2-Diphytanoyl-sn-glycero-3-phosphocholine (DphPC) was used to form a self-assembled lipid bilayer sealing the orifice. This lipid bilayer divides the chamber into cis and trans compartments both filled with 0.5 mL of 1.5 M KC1 buffer (1.5 M KC1, 10 mM Tris-HCl, pH 7.0). A pair of Ag/AgCl electrodes were placed in cis and trans side of the chamber, in contact with the aqueous buffer respectively. Biological nanopores (WT a-HL, α-HL M113G, M2 MspA or MspA-M) were added to cis for spontaneous pore insertion.

For [AuCl₄]⁻¹ binding experiments, the acquired single channel data was sampled at 25 kHz and filtered with a corner frequency of 1 kHz. For [AuCl₄]⁻¹ binding events, the recorded current traces were digitally filtered with a 200 Hz low-pass Bessel filter (eight-pole) and the events were detected by the single-channel search feature in Clampfit 10.7 (Axon Instruments).

For ssDNA translocation experiments, the data was sampled at 250 kHz and filtered with a corner frequency of 100 kHz. For ssDNA translocation events, the recorded current traces were digitally filtered with a 10 kHz low-pass Bessel filter (eight-pole) and the events were detected by the single-channel search feature in Clampfit 10.7 (Axon Instruments). Further analyses (histogram, curve fitting and plotting) were carried out in Origin 9.1 (Origin Lab).

For specific sensing of L-methionine, the data was sampled at 25 kHz and filtered with a corner frequency of 1 kHz. Event states 1-3 were detected by the single channel search feature in Clampfit 10.7. Further analysis was carried out in Origin 9.1.

For specific sensing of L-glutamic aicd L-homocysteine and L-glutathione, all measurements were performed with a +100 mV continuously applied voltage. The acquired single channel data was sampled at 25 kHz and filtered with a corner frequency of 1 kHz. The recorded current traces were digitally filtered with a 0.2 kHz low-pass Bessel filter (eight-pole). Event states were detected by the single channel search feature in Clampfit 10.7 and further analyses (histogram, curve fitting and plotting) were carried out in Origin 9.1 (Origin Lab).

### REFERENCES

1. Jain, M., Olsen, H. E., Paten, B. & Akeson, M. The Oxford Nanopore MinION: delivery of nanopore sequencing to the genomics community. Genome biology 17, 239 (2016).
2. Cao, C. et al. Discrimination of oligonucleotides of different lengths with a wild-type aerolysin io nanopore. Nature nanotechnology 11, 713-718 (2016).
3. Laszlo, A. H. et al. Decoding long nanopore sequencing reads of natural DNA. Nature biotechnology 32, 829-833 (2014).
4. Manrao, E. A. et al. Reading DNA at single-nucleotide resolution with a mutant MspA nanopore and phi29 DNA polymerase. Nature biotechnology 30, 349-353 (2012).
5. Wallace, E .V. et al. Identification of epigenetic DNA modifications with a protein nanopore. Chemical communications 46, 8195-8197 (2010).
6. Ayub, M., Hardwick, S. W., Luisi, B .F. & Bayley, H. Nanopore-based identification of individual nucleotides for direct RNA sequencing. Nano letters 13, 6144-6150 (2013).
7. Sutherland, T.C. et al. Structure of peptides investigated by nanopore analysis. Nano letters 4, 1273-1277 (2004).
8. Nivala, J., Marks, D. B. & Akeson, M. Unfoldase-mediated protein translocation through an α-hemolysin nanopore. Nature biotechnology 31, 247-250 (2013).
9. Song, L. et al. Structure of staphylococcal a-hemolysin, a heptameric transmembrane pore. Science 274, 1859-1865 (1996).
10. Braha, O. et al. Designed protein pores as components for biosensors. Chemistry & Biology 4, 497-505 (1997).
11. Mayne, L. J., Christie, S. D. R. & Platt, M. A tunable nanopore sensor for the detection of metal ions using translocation velocity and biphasic pulses. Nanoscale 8, 19139-19147 (2016).
12. Braha, O. et al. Simultaneous stochastic sensing of divalent metal ions. Nature biotechnology 18, 1005-1007 (2000).
13. Choi, L. S., Mach, T. & Bayley, H. Rates and stoichiometries of metal ion probes of cysteine residues within ion channels. Biophysical journal 105, 356-364 (2013).
14. Hammerstein Anne , F., Shin, S. H. & Bayley, H. Single - Molecule Kinetics of Two-Step Divalent Cation Chelation. Angewandte Chemie International Edition 49, 5085-5090 (2010).
15. Wen, S. et al. Highly sensitive and selective DNA-based detection of mercury(II) with alpha-hemolysin nanopore. Journal of the American Chemical Society 133, 18312-18317 (2011).
16. Roozbahani, G. M. et al. Peptide-Mediated Nanopore Detection of Uranyl Ions in Aqueous Media. ACS Sensors 2, 703-709 (2017).
17. Guo, Y, Jian, F. & Kang, X. Nanopore sensor for copper ion detection using a polyamine decorated β-cyclodextrin as the recognition element. RSC Advances 7, 15315-15320 (2017).
18. Sun, Y. & Xia, Y. Mechanistic study on the replacement reaction between silver nanostructures and chloroauric acid in aqueous medium. Journal of the American Chemical Society 126, 3892-3901 (2004).
19. Shankar, S. S., Rai, A., Ahmad, A. & Sastry, M. Rapid synthesis of Au, Ag, and bimetallic Au core-Ag shell nanoparticles using Neem (Azadirachta indica) leaf broth. Journal of colloid and interface science 275, 496-502 (2004).
20. Williams, J. M. & Peterson, S. W. Example of the [H5O2]+ ion. Neutron diffraction study of tetrachloroauric acid tetrahydrate. Journal of the American Chemical Society 91, 776-777 (1969).
21. Niemietz, C. M. & Tyerman, S. D. New potent inhibitors of aquaporins: silver and gold compounds inhibit aquaporins of plant and human origin. FEBS letters 531, 443-447 (2002).
22. Butler, T. Z., Pavlenok, M., Derrington, I. M., Niederweis, M. & Gundlach, J. H. Single-molecule DNA detection with an engineered MspA protein nanopore. Proceedings of the National Academy of Sciences 105, 20647-20652 (2008).
23. Derrington, I. M. et al. Nanopore DNA sequencing with MspA. Proceedings of the National

Academy of Sciences 107, 16060-16065 (2010).
24. Porter, M. D., Bright, T. B., Allara, D. L. & Chidsey, C. E. Spontaneously organized molecular assemblies. 4. Structural characterization of n-alkyl thiol monolayers on gold by optical ellipsometry, infrared spectroscopy, and electrochemistry. Journal of the American Chemical Society 109, 3559-3568 (1987).
25. Ivanova, B. B. & Mitewa, M. I. Au(III) interaction with Methionine- and Histidine-containing peptides. Journal of Coordination Chemistry 57, 217-221 (2004).
26. Vujaeie, A. V. et al. Mechanism of complex formation between [AuCl_{4]}- and 1-methionine. Polyhedron 28, 593-599 (2009).
27. A1-maythalony, B., Wazeer, M., Isab, A. & Ahmad, S. A study of [Au(ethylenediamine)C12]C1 interaction with L-methionine and DL-seleno-methionine by 1H and 13C NMR spectroscopy. Spectroscopy 24, 567-575 (2010). 28. Stoddart, D., Maglia, G., Mikhailova, E., Heron, A. J. & Bayley, H. Multiple Base-Recognition Sites in a Biological Nanopore: Two Heads are Better than One. Angewandte Chemie International Edition 49, 556-559 (2010).
29. Goyal, P. et al. Structural and mechanistic insights into the bacterial amyloid secretion channel CsgG. Nature 516, 250 (2014).
30. Wang, Y. et al. Osmosis-Driven Motion-Type Modulation of Biological Nanopores for Parallel Optical Nucleic Acid Sensing. ACS applied materials & interfaces 10, 7788-7797 (2018).
31. Faller, M., Niederweis, M. & Schulz, G.E. The structure of a mycobacterial outer-membrane channel. Science 303, 1189-1192 (2004).
32. Rincon-Restrepo, M., Mikhailova, E., Bayley, H. & Maglia, G. Controlled translocation of individual DNA molecules through protein nanopores with engineered molecular brakes. Nano letters 11, 746-750 (2011).
33. Franceschini, L., Brouns, T., Willems, K., Carlon, E. & Maglia, G. DNA Translocation through Nanopores at Physiological Ionic Strengths Requires Precise Nanoscale Engineering. Acs Nano 10, 8394-8402 (2016).
34. Maglia, G., Restrepo, M. R., Mikhailova, E. & Bayley, H. Enhanced translocation of single DNA molecules through alpha-hemolysin nanopores by manipulation of internal charge. Proceedings of the National Academy of Sciences of the United States of America 105, 19720-19725 (2008).
35. Restrepo-Perez, L., Joo, C. & Dekker, C. Paving the way to single-molecule protein sequencing. Nature nanotechnology 13, 786-796 (2018).
36. Boersma, A.J. & Bayley, H. Continuous stochastic detection of amino acid enantiomers with a protein nanopore. Angewandte Chemie 124, 9744-9747 (2012).
37. Boersma, A.J., Brain, K.L. & Bayley, H. Real-time stochastic detection of multiple neurotransmitters with a protein nanopore. Acs Nano 6, 5304-5308 (2012).
38. Chen, M., Khalid, S., Sansom, M.S. & Bayley, H. Outer membrane protein G: Engineering a quiet pore for biosensing. Proceedings of the National Academy of Sciences 105, 6272-6277 (2008).
39. Fahie, M., Chisholm, C. & Chen, M. Resolved single-molecule detection of individual species within a mixture of anti-biotin antibodies using an engineered monomeric nanopore. Acs Nano 9, 1089-1098 (2015).
40. Perez-Rathke, A., Fahie, M .A., Chisholm, C., Liang, J. & Chen, M. Mechanism of OmpG pH-dependent gating from loop ensemble and single channel studies. Journal of the American Chemical Society 140, 1105-1115 (2018).
41. Soskine, M. et al. An engineered ClyA nanopore detects folded target proteins by selective external association and pore entry. Nano letters 12, 4895-4900 (2012).
42. Wendell, D. et al. Translocation of double-stranded DNA through membrane-adapted phi29 motor protein nanopores. Nature nanotechnology 4, 765-772 (2009).
43. Brust, M., Walker, M., Bethell, D., Schiffrin, D.J. & Whyman, R. Synthesis of thiol-derivatised gold nanoparticles in a two-phase liquid-liquid system. Journal of the Chemical Society, Chemical Communications, 801-802 (1994).
44. Huang, S. et al. Identifying single bases in a DNA oligomer with electron tunnelling. Nature nanotechnology 5, 868-873 (2010).
45. Dasary, S. S., Singh, A. K., Senapati, D., Yu, H. & Ray, P. C. Gold nanoparticle based label-free SERS probe for ultrasensitive and selective detection of trinitrotoluene. Journal of the American Chemical Society 131, 13806-13812 (2009).
46. Anker, J. N. et al. Biosensing with plasmonic nanosensors. Nature materials 7, 442-453 (2008).
47. Wu, H.-C., Astier, Y, Maglia, G., Mikhailova, E. & Bayley, H. Protein nanopores with covalently attached molecular adapters. Journal of the American Chemical Society 129, 16142-16148 (2007).
48. Butler, T. Z., Gundlach, J. H. & Troll, M. A. Determination of RNA orientation during translocation through a biological nanopore. Biophysical journal 90, 190-199 (2006).
49. Peng, Y. Y., Ma, H., Ma, W, Long, Y. T. & Tian, H. Single-Nanoparticle Photoelectrochemistry at a

Nanoparticulate TiO2 - Filmed Ultramicroelectrode. Angewandte Chemie International Edition 57, 3758-3762 (2018).
50. Ying, Y-L. et al. Asymmetric Nanopore Electrode-Based Amplification for Electron Transfer

Imaging in Live Cells. Journal of the American Chemical Society 140, 5385-5392 (2018).
51. Wang Y, Yan S, Zhang P, Zeng Z, Zhao D, Wang J, et al. Osmosis-Driven Motion-Type Modulation of Biological Nanopores for Parallel Optical Nucleic Acid Sensing. ACS applied materials & interfaces 2018, 10(9): 7788-7797.
52. Larkin J, Henley R Y, Jadhav V, Korlach J, Wanunu M. Length-independent DNA packing into nanopore zero-mode waveguides for low-input DNA sequencing. Nature nanotechnology 2017, 12(12): 1169.
53. Durovic M D, Bugarcic Z D, Heinemann F W, van Eldik R. Substitution versus redox reactions of gold(III) complexes with L-cysteine, L-methionine and glutathione. Dalton transactions 43, 3911-3921 (2014).
54. Dalton T P, Shertzer H G, Puga A. REGULATION OF GENE EXPRESSION BY REACTIVE OXYGEN. Annual Review of Pharmacology and Toxicology 39, 67-101 (1999).
55. Brandes N, Schmitt S, Jakob U. Thiol-based redox switches in eukaryotic proteins. Antioxid Redox Signal 11, 997-1014 (2009).
56. Gregory IG. The Redox Regulation of Thiol Dependent Signaling Pathways in Cancer. Current Pharmaceutical Design 12, 4427-4443 (2006).
57. Gahl W A, Bashan N, Tietze F, Bernardini I, Schulman J. Cystine transport Is defective in isolated leukocyte lysosomes from patients with cystinosis (1982).
58. Lu SC. Regulation of glutathione synthesis. Molecular aspects of medicine 30, 42-59 (2009).
59. Yin C X, Xiong K M, Huo FJ, Salamanca JC, Strongin RM. Fluorescent Probes with Multiple Binding Sites for the Discrimination of Cys, Hcy, and GSH. Angew Chem Int Ed Engl 56, 13188-13198 (2017).
60. Seiwert B, Karst U. Simultaneous LC/MS/MS determination of thiols and disulfides in urine samples based on differential labeling with ferrocene-based maleimides. Analytical chemistry 79, 7131-7138 (2007).
61. Zhang H, et al. A minimalist fluorescent probe for differentiating Cys, Hcy and GSH in live cells. Chem Sci 7, 256-260 (2016).
62. MacCoss MJ, Fukagawa NK, Matthews DE. Measurement of Homocysteine Concentrations and Stable Isotope Tracer Enrichments in Human Plasma. Analytical chemistry 71, 4527-4533 (1999).
63. Sachdev P, et al. Homocysteine and the Brain in Midadult Life: Evidence for an Increased Risk of Leukoaraiosis in Men. JAMA Neurology 61, 1369-1376 (2004).
64. R. G. Pearson, Hard and Soft Acids and Bases. Journal of the American Chemical Society 85, 3533-3539 (1963).

1-88. (canceled)

89. Method of identifying a metal-containing analyte in a sample, the method comprising:
providing a MspA positioned between a first conductive liquid medium and a second conductive liquid medium, wherein the MspA comprises an opening that provides liquid communication between the first conductive liquid medium and the second conductive liquid medium, wherein the first conductive liquid medium or the second conductive liquid medium comprises the sample;
applying an electric field across the MspA and translocating the metal-containing ion through the nanopore;
measuring the blockade current across the nanopore; and
identifying the metal-containing ion in the sample according to the measured blockade current.

90. The method of claim 89, wherein the metal-containing analyte is a metal-containing ion.

91. The method of claim 90, wherein the metal-containing ion are selected from the group consisting of ions contain Au(III), Zn²⁺, Co²⁺, Ag⁺, Cd²⁺, Ni²⁺, Au(I), Cu²⁺, Cr³⁺, Fe²⁺, Fe³⁺, Pt(II), Pd(II), Mn^{2 +}, Hg²⁺, Ru(II) and Pb^{2 +}.

92. The method of claim 90, wherein the MspA is a mutant octameric MspA, said mutant octameric MspA comprises at least one mutant MspA monomers, said mutant octameric MspA has one or more amino acid residues which can interact with the metal ion on the inner surface of the nanopore channel; wherein at least one of the mutant MspA monomers comprises one or more mutations at positions 83-111 compared to the wild-type MspA monomer.

93. The method of claim 92, wherein the one or more mutations at positions 83-111 may be one or more mutations at position 88, 89, 90, 91, 92, 93, 94, 95, 96, 97, 98, 99, 100, 101, 102, 103, 104 and/or 105.

94. The method of claim 92, wherein said mutation is a mutation to methionine, cysteine or histidine.

95. The method of claim 92, wherein at least one of the mutant MspA monomers comprises the mutation of D91M, D91H or D91C compared to the wild-type MspA monomer.

96. Method of identifying an analyte in a sample, the method comprising:
providing a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample, wherein the nanopore is embedded with one or more metal-containing ions;
applying an electric field across the nanopore and translocating the analyte through the nanopore;
measuring the blockade current across the nanopore; and
identifying the analyte in the sample according to the measured blockade current;
wherein the protein nanopore is MspA, CsgG, OmpG, ClyA, aerolysin, FraC or Phi29 connector.

97. The method of claim 96, wherein the one or more metal-containing ions are selected from the group consisting of ions contain Au(III), Zn²⁺, Co²⁺, Ag⁺, Cd²⁺, Ni²⁺, Au(I), Cu²⁺, Cr³⁺, Fe²⁺, Fe³⁺, Pt(II), Pd(II), Hg²⁺, Ru(II) and Pb²⁺.

98. The method of claim 96, wherein MspA is a mutant octameric MspA, said mutant octameric MspA comprises at least one mutant MspA monomers, said mutant octameric MspA has one or more amino acid residues which can interact with the metal ion on the inner surface of the nanopore channel; and wherein at least one of the mutant MspA monomers comprises one or more mutations at positions 83-111 compared to the wild-type MspA monomer.

99. The method of claim 98, wherein the one or more mutations at positions 83-111 are one or more mutations at position 88, 89, 90, 91, 92, 93, 94, 95, 96, 97, 98, 99, 100, 101, 102, 103, 104 and/or 105.

100. The method of claim 98, wherein said mutation is a mutation to methionine, cysteine or histidine.

101. The method of claim 98, wherein at least one of the mutant MspA monomers comprises the mutation of D91M, D91H or D91C compared to the wild-type MspA monomer.

102. The method of claim 96, wherein the analyte is metal-containing analyte, nucleic acid, amino acid, peptide or thiol.

103. System of identifying an analyte in a sample, the system contains a protein nanopore positioned between a first conductive liquid medium and a second conductive liquid medium, wherein the nanopore comprises an opening that provides liquid communication between the first conductive liquid medium and the second conductive liquid medium, wherein the first conductive liquid medium or the second conductive liquid medium comprises the sample, wherein the nanopore is embedded with one or more metal-containing ions;
wherein the protein nanopore is MspA, CsgG, OmpG, ClyA, aerolysin, FraC or Phi29 connector.

104. The system of claim 103, wherein the one or more metal-containing ions are selected from the group consisting of ions contain Au(III), Zn²⁺, Co²⁺, Ag⁺, Cd²⁺, Ni²⁺, Au(I), Cu²⁺, Cr³⁺, Fe²⁺, Fe³⁺, Pt(II), Pd(II), Mn²⁺, Hg²⁺, Ru(II) and Pb²⁺.

105. The system of claim 103, wherein MspA is a mutant octameric MspA, said mutant octameric MspA comprises at least one mutant MspA monomers, said mutant octameric MspA has one or more amino acid residues which can interact with the metal ion on the inner surface of the nanopore channel; and wherein at least one of the mutant MspA monomers comprises one or more mutations at positions 83-111 compared to the wild-type MspA monomer.

106. The system of claim 105, wherein the one or more mutations at positions 83-111 are one or more mutations at position 88, 89, 90, 91, 92, 93, 94, 95, 96, 97, 98, 99, 100, 101, 102, 103, 104 and/or 105.

107. The system of claim 105, wherein said mutation is a mutation to methionine, cysteine or histidine.

108. The system of claim 105, wherein at least one of the mutant MspA monomers comprises the mutation of D91M, D91H or D91C compared to the wild-type MspA monomer.

109. The system of claim 103, wherein the analyte is metal-containing analyte or nucleic acid, amino acid, peptide or thiol.
